(19) United States
(12) Patent Application Publication
(10) Pub. No.: US 2026/0188496 A1
(43) Pub. Date: Jul. 2, 2026

(54) SYSTEMS AND METHODS FOR PROCESSING DATA AMONG MULTIPLE NODES

(71) Applicant: Direct Supply, Inc., Milwaukee, WI (US)

(72) Inventors: Lee Jones, Crestwood, KY (US); Adam Tobbe, Crestwood, KY (US); Dustin Shilala, DuBois, PA (US)

(21) Appl. No.: 19/435,437

(22) Filed: Dec. 29, 2025

Related U.S. Application Data
(60) Provisional application No. 63/739,409, filed on Dec. 27, 2024.

(51) Int. Cl. G16H 50/20

(52) U.S. Cl. CPC G16H 50/20

(57) ABSTRACT

The document relates to systems and methods for machine learning based data processing among nodes. An exemplary computer-implemented method includes receiving, via the network interface, an electronic data package; obtaining a document image derived from the electronic data package; generating extracted text and location data from the document image, the location data defining mapped locations of the extracted text within the document image; generating, using a machine learning component, a structured response including at least one compatibility indicator and a source citation; determining whether the at least one compatibility indicator satisfies the node capability specification of the service providing node; and generating a verification interface including a visual overlay visually associating, based on at least one of the source citation or the mapped location of the at least one relevant text chunk, the at least one compatibility indicator with the at least one relevant text chunk on the document image.

### CROSS-REFERENCE TO RELATED APPLICATIONS

The application claims the benefit of and priority to U.S. Provisional Application No. 63/739,409, filed Dec. 27, 2024, the disclosure of which is incorporated herein by reference in its entirety.

### BACKGROUND

The referral process from hospitals to post-acute care (PAC) communities involves assessing the patient's medical needs, developing a care plan, and identifying suitable PAC providers.

### SUMMARY

An aspect of the present document relates to a system. The system includes a network interface configured to receive input data streams from multiple source nodes; at least one processor coupled to the network interface; memory coupled to the at least one processor, wherein the memory stores instructions that, when executed by the at least one processor, cause the at least one processor to perform operations including: receiving, via the network interface, an electronic data package from a source node; obtaining a document image derived from the electronic data package; generating extracted text and location data from the document image, the location data defining mapped locations of the extracted text within the document image; generating, using a machine learning (ML) component, a structured response by: generating a prompt payload comprising at least a portion of the extracted text and a node capability specification of a service providing node; providing the prompt payload to a generative AI model of the ML component; and receiving the structured response from the generative AI model, wherein the structured response comprises: (i) at least one compatibility indicator determined based on the capability specification of the service providing node, and (ii) a source citation identifying at least one relevant text chunk from the extracted text that supports the at least one compatibility indicator; determining whether the at least one compatibility indicator satisfies the node capability specification of the service providing node; and generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

Another aspect of the present document relates to a computer-implemented method. The method includes receiving, by at least one processor via a network interface, an electronic data package from a source node; obtaining a document image derived from the electronic data package; generating extracted text and location data from the document image, the location data defining mapped locations of the extracted text within the document image; generating, using a machine learning (ML) component, a structured response by: generating a prompt payload comprising at least a portion of the extracted text and a node capability specification of a service providing node; providing the prompt payload to a generative AI model of the ML component; and receiving the structured response from the generative AI model, wherein the structured response comprises: (i) at least one compatibility indicator determined based on the node capability specification of the service providing node, and (ii) a source citation identifying at least one relevant text chunk from the extracted text that supports the at least one compatibility indicator; determining whether the at least one compatibility indicator satisfies the node capability specification of the service providing node; and generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

A further aspect of the present document relates to at least one non-transitory computer-readable storage medium storing instructions that, when executed by at least one processor, cause the at least one processor to perform the methods disclosed in the present document.

A still further aspect of the present document relates to a system, including memory storing computer-readable instructions; one or more processors that when executing the computer-readable instructions, are configured to perform the methods disclosed in the present document.

### BRIEF DESCRIPTION OF THE DRAWINGS

FIG. 1A illustrates a many-to-many relationship between referral sources to post-acute communities, and for each community the steps to process each referral.

FIG. 1B illustrates a centralized data processing architecture, according to some embodiments of the present technology.

FIG. 2A illustrates a flowchart of a process performed by a computer-implemented data processing system, according to some embodiments of the present technology.

FIG. 2B illustrates a flowchart of a process performed by a computer-implemented data processing system, according to some embodiments of the present technology.

FIG. 2C illustrates a flowchart of a process performed by a computer-implemented data processing system, according to some embodiments of the present technology.

FIG. 3A illustrates a flowchart of a process performed by a computer-implemented data processing system, according to some embodiments of the present technology.

FIG. 3B illustrates a data processing system, according to some embodiments of the present technology.

FIG. 4 illustrates a system block diagram of a centralized processing hub or system, according to some embodiments of the present technology.

FIG. 5 illustrates a flow diagram for a community capability mapping, according to some embodiments of the present technology.

FIG. 6 illustrates a flow diagram of a data ingestion and standardization pipeline, according to some embodiments of the present technology.

FIG. 7 illustrates an exemplary AI custom-answer workflow, according to some embodiments of the present technology.

FIG. 8 illustrates a diagram for LLM clinical question answering, according to some embodiments of the present technology.

FIG. 9A shows an exemplary LLM answer output in response to prompt and provided referral information, according to some embodiments of the present technology.

FIG. 9B illustrates example clinical questions and answers including icons indicating LLM answered, and LLM answered/user edited, according to some embodiments of the present technology.

FIG. 10 illustrates an architecture diagram for retrieval-augmented generation (RAG) integration, according to some embodiments of the present technology.

FIG. 11 illustrates a workflow for providing answers to clinical questions along with specific citations to referral information, according to some embodiments of the present technology.

FIG. 12 illustrates a flow diagram for a document-to-AI positional correlation and visual highlighting system, according to some embodiments of the present technology.

FIGS. 13A and 13B illustrate exemplary user interfaces showing correlation between AI generated answers and information extracted from a referral packet, according to some embodiments of the present technology.

FIG. 14 illustrates a flow diagram for capability matching via direct database correlations, according to some embodiments of the present technology.

FIG. 15 illustrates an exemplary patient referral profile interface, according to some embodiments of the present technology.

FIGS. 16 and 17 show exemplary user interfaces including map visualizations, according to some embodiments of the present technology.

FIG. 18 is a flow diagram illustrating an exemplary LACE score system workflow, according to some embodiments.

FIGS. 19 and 20 show exemplary user interfaces including LACE scores, according to some embodiments.

FIG. 21 is a flow diagram illustrating an exemplary AI-powered PDPM calculation workflow, according to some embodiments.

FIG. 22 shows an exemplary user interface for a PDPM calculator, according to some embodiments.

FIG. 23 is a user interface showing exemplary PDPM rates by day, according to some embodiments.

FIG. 24 is a flow diagram illustrating an exemplary medication cost RAG system workflow, according to some embodiments.

FIGS. 25 and 26 show exemplary user interfaces including medication information, according to some embodiments.

FIG. 27 shows an exemplary user interface illustrating a comprehensive referral scorecard, according to some embodiments.

FIG. 28 illustrates a flow diagram illustrating an exemplary AI-powered medical equipment (ME) RAG system workflow, according to some embodiments.

FIG. 29 is a flow diagram illustrating an exemplary basic rules workflow 2900, according to some embodiments.

FIGS. 30-32 show exemplary user interfaces for rules input and configuration, according to some embodiments.

FIG. 33 illustrates a flow diagram of a population monitoring workflow 3300, according to some embodiments.

FIG. 34 is a block diagram that illustrates an example AI system that can implement aspects of the present technology.

FIG. 35 is a block diagram that illustrates an example of a computer system in which at least some operations described herein can be implemented.

FIGS. 36-38 illustrate various views of the user interface, according to some embodiments of the present technology.

FIG. 39 illustrates a process for capability matching between referral needs and community capabilities, according to some embodiments of the present technology.

FIGS. 40A-40C are views of a clinical question, clinical review, and community review, according to some embodiments of the present technology.

FIG. 41A is a view of a clinical review showing a referral packet and clinical review questions, according to some embodiments of the present technology.

FIG. 41B is a view of a clinical review submission modal illustrating a submission and denial for two different communities, according to some embodiments of the present technology.

FIG. 42A shows a community review workflow, according to some embodiments of the present technology.

FIG. 42B is a view of a community review summary for a referral under Q&A, according to some embodiments of the present technology.

FIGS. 43A and 43B show views of a referral accepted by a community, according to some embodiments of the present technology.

FIG. 44 is a view of an example high risk admit referral alert to a user, according to some embodiments of the present technology.

FIG. 45A is a grid view of referrals showing referrals pending denial review, according to some embodiments of the present technology.

FIG. 45B is a denial management user view of specific referral being reviewed, according to some embodiments of the present technology.

FIG. 45C is a grid view showing referrals that have had denials overturned returned to workflow, according to some embodiments of the present technology.

FIGS. 45D and 45E are views showing modals for entering explanation and confirming denial rejection, according to some embodiments of the present technology.

FIG. 46 shows a process for identifying and recommending suitable communities for referrals, according to some embodiments of the present technology.

FIG. 47 is a view of dashboard overview, according to some embodiments of the present technology.

FIGS. 48A-48C are various views of a patient referral workflow, according to some embodiments of the present technology.

FIG. 49 is a view of a dashboard, according to some embodiments of the present technology.

The headings provided herein are for convenience only and do not necessarily affect the scope of the embodiments. Further, the drawings have not necessarily been drawn to scale. For example, the dimensions of some of the elements in the figures may be expanded or reduced to help improve the understanding of the embodiments. Moreover, while the disclosed technology is amenable to various modifications and alternative forms, specific embodiments have been shown by way of example in the drawings and are described in detail below. The embodiments are intended to cover all suitable modifications, combinations, equivalents, and alternatives falling within the scope of this disclosure.

### DETAILED DESCRIPTION

Various examples of the systems and methods introduced above will now be described in further detail. The following description provides specific details for a thorough understanding and enabling description of these examples. One skilled in the relevant art will understand, however, that the techniques and technology discussed herein may be practiced without many of these details. Likewise, one skilled in the relevant art will also understand that the technology can include many other features not described in detail herein. Additionally, some well-known structures or functions may not be shown or described in detail below so as to avoid unnecessarily obscuring the relevant description.

The referral process from source nodes (e.g., hospitals) to service providing nodes (e.g., post-acute care (PAC) communities) involves assessing the patient's medical needs, developing a care plan, and identifying suitable providers. Referrals are often made to multiple PAC communities, each taking several steps to process the referral, including referral capture, clerical activities, clinical review, site-specific determinations, and the conversion process. Typically, the hospital submits the referral via a data package sharing detailed patient information (often in unstructured formats like PDF or fax images), and the PAC providers review it to determine if they can meet the patient's needs. If accepted, the hospital coordinates the transfer with the community.

The prior referral intake process for post-acute long-term care from hospitals has many inefficiencies and complexities. Time is critical, as typically, the first community to accept a referral gets it, and delays can cause lost admissions. There are additional time and labor costs associated with processing referrals. Time spent processing referrals takes away from time nurses can focus on providing quality care. In addition, even if there is a central intake team this takes costly dedicated staff. Technically, the manual or semi-automated processing of these unstructured data packages at individual nodes introduces significant network latency and processing overhead. Additionally, the lack of a uniform center of truth for data and reporting exacerbates the problem and increases the risk of poorly vetted admissions. The use of multiple disparate systems further heightens the likelihood of errors (e.g., data entry errors), making the process even more prone to mistakes. On top of it all, each community within an organization may repeat the same steps (e.g., extracting data, analyzing constraints) in processing a referral.

Section headings are used in the present document to improve readability of the description and do not in any way limit the discussion or the embodiments (and/or implementations) to the respective sections only.

### I. System Overview & Architecture

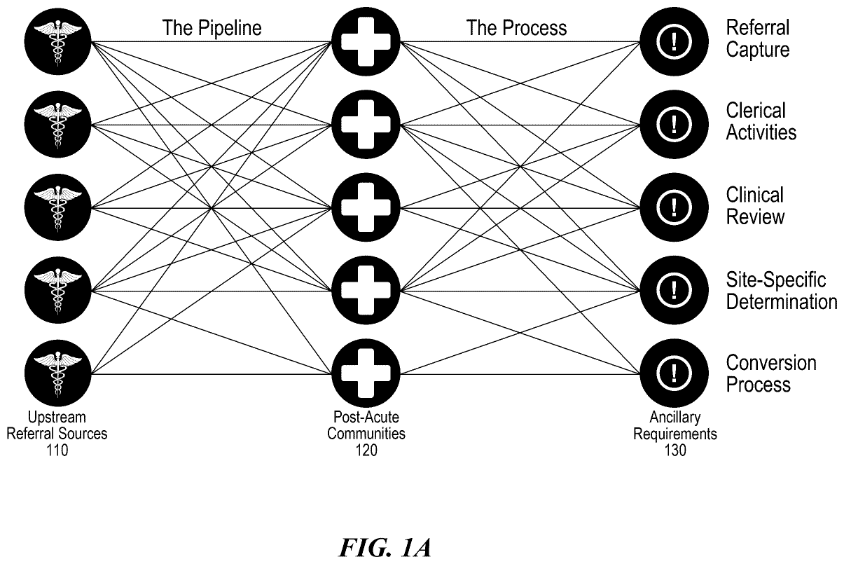

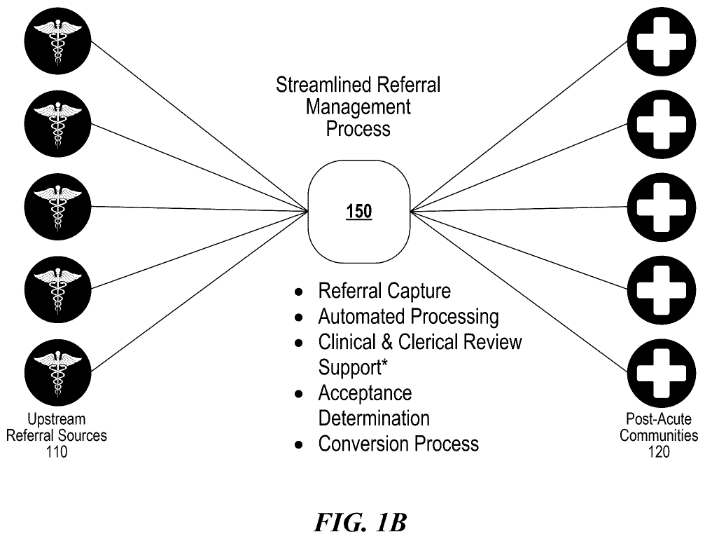

FIG. 1A illustrates a many-to-many relationship between referral sources to post-acute communities, and for each community the steps to process each referral. These steps are often duplicated at multiple referred communities in an organization. As illustrated, multiple referral sources (or source nodes) 110 (shown on the left) are connected to multiple post-acute communities 120 (shown in the center), with each post-acute community performing a sequence of operations 130 (shown on the right). The operations include data capture (e.g., ingestion), administrative processing, analysis review, node-specific determinations, and data transformation procedures. The interconnected network demonstrates many-to-many relationships between referral sources 110 and post-acute communities 120, with each post-acute community 120 independently executing the complete operation sequence 130.

An “organization” may refer to a parent entity or tenant (e.g., a healthcare company) that manages the overall system usage. In the system architecture, high-level data rules—such as insurance contracts and medication formularies—may be set at the organization level to ensure consistency and data privacy across the tenant's network. A “community” refers to a healthcare facility, a care center, a tenant, or a specific physical location where patient care is delivered (e.g., a specific care facility). An organization may include multiple communities. In the context of the system's decision-making, physical constraints—such as bed availability or specialized equipment—may be defined at the community level to determine if a specific site can admit a patient.

The many-to-many relationships shown in FIG. 1A create several technical challenges in distributed processing systems. The parallel processing of identical data packages across multiple processing nodes results in redundant computations and duplicated analysis steps. For example, multiple nodes may redundantly perform optical character recognition (OCR) or manual review on the same document image. This distributed nature, where each node independently processes data, creates data consistency issues across the system, leading to potential inconsistencies where different nodes interpret the same patient data differently.

This architecture leads to inefficient resource utilization through multiple mechanisms. Multiple nodes perform identical processing steps independently, creating computational redundancy. Processing delays impact timely data distribution across nodes, while resource-intensive operations are duplicated throughout the network. The lack of uniform data management creates consistency challenges, and the use of multiple independent systems increases the probability of processing errors. Manual processing steps introduce additional inefficiencies in the system workflow, preventing the rapid “hard stop” filtering required for patient safety.

The system architecture takes dedicated processing resources at each node while lacking centralized data management and standardized processing workflows. The absence of a unified data repository (e.g., a centralized vector store index) prevents consistent data access across nodes. Without coordinated processing approaches, the system experiences increased overhead and potential data discrepancies between processing nodes.

The technology disclosed herein aims to streamline and optimize the data processing among multiple nodes (in an example use case, a referral intake process from hospitals to post-acute care (PAC) communities) by leveraging automation, integration, and centralized data management. The system centralizes the referral management process of upstream referral sources for post-acute communities minimizing redundancy within an organization and turning around referral processing quicker. This results in a significant reduction in wasted labor and an increased referral win chance. It also mitigates against communities skipping steps in the process and admitting patients inappropriate to the community (e.g., due to mismatched patient needs and community capabilities). By utilizing a centralized processing hub that executes OCR, machine learning analysis, and rule-based matching once, the system improves or ensures data integrity and operational efficiency.

FIG. 1B illustrates a centralized data processing architecture, according to some embodiments of the present technology. The centralized data process architecture illustrated in FIG. 1B transforms the many-to-many relationships into a hub-and-spoke model. Multiple referral sources 110 (depicted on the left) connect to a referral management system 150 including a central processing hub (or at least one processor configured to act as such), which then connects to multiple PACs 120 (shown on the right).

The centralized architecture implements features that address data management challenges. It provides a single source of truth for all system data, enabling consistent information access across all nodes. The system includes intuitive dashboarding and reporting capabilities for system monitoring and analysis. It aggregates multiple referral channels into a unified processing stream, standardizing data handling while reducing resource needs through centralized processing. In some embodiments, this involves converting unstructured inputs into structured analysis records using vector embeddings and pairwise correlation determinations, ensuring that downstream nodes receive validated, structured data.

The referral management system 150 implements features for efficient operation. It provides role-defined workflow management to control data access and processing steps. The system automates and simplifies processing operations through its centralized architecture. It performs intelligent matching of referrals to site capabilities through capability analysis (e.g., comparing compatibility indicators against node capability specifications). The system enables a verification interface that allows users to spatially verify the extracted data against the original document image. The system enables real-time, secure communication between connected nodes while maintaining compliance requirements (e.g., via data sanitization and masking).

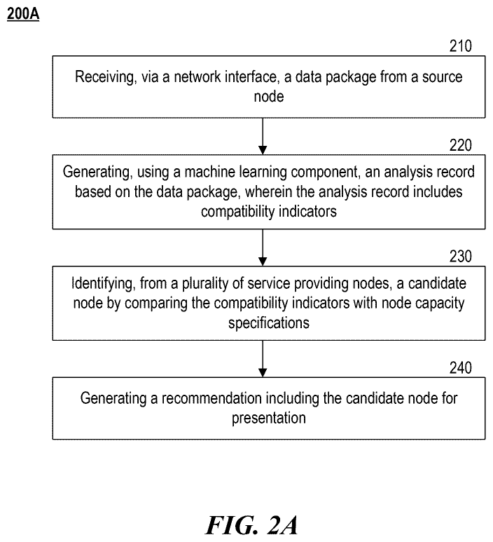

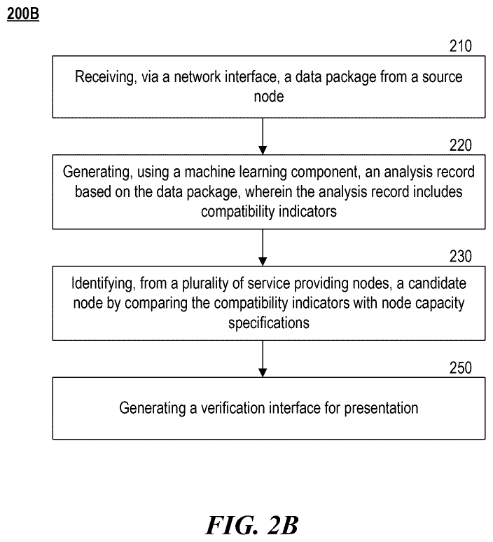

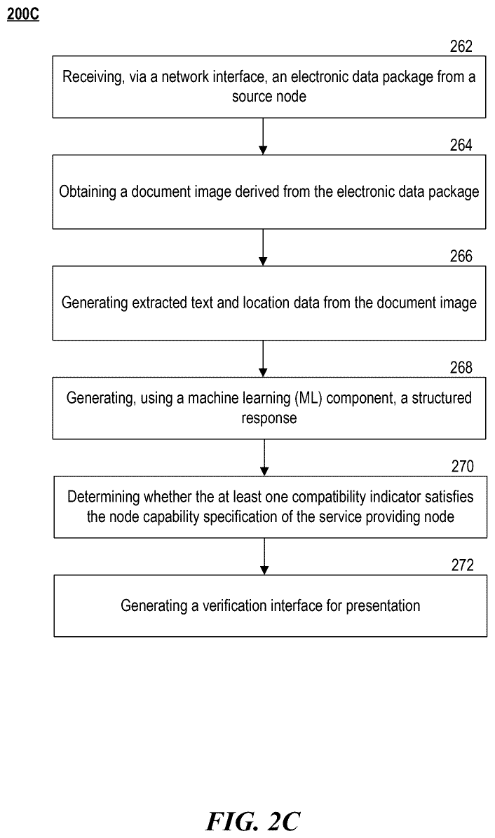

This centralized architecture enables unified data processing and standardized operations across all connected nodes, reducing redundant processing while maintaining connections between multiple sources and destinations. The centralized data processing architecture may be implemented according to the processes 200A, 200B, and 200C as illustrated in FIGS. 2A-2C, and utilizing the specific engines (e.g., OCR, embedding models, and visual verification rendering) detailed herein.

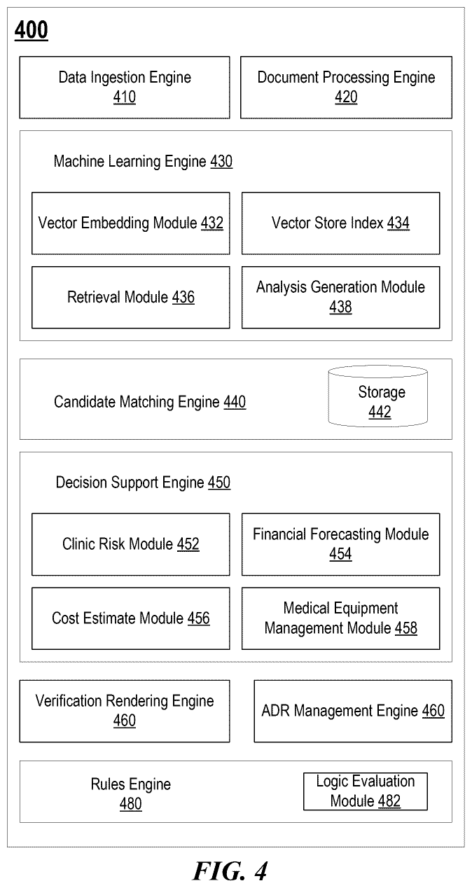

FIG. 2A illustrates a flowchart of a process 200A performed by a computer-implemented data processing system, according to some embodiments of the present technology. FIGS. 2B and 2C illustrate a flowchart of processes 200B and 200C, respectively, performed by the system, including the generation of a verification interface. The process 200A/200B/200C may be performed by the referral management system 150 or system 300B to implement the centralized data processing architecture, utilizing functional engines (e.g., Data Ingestion Engine, Document Processing Engine) illustrated in the system block diagram of FIG. 4.

At 210, the process 200A/200B includes receiving a data package (e.g., an electronic data package) from a source node via a network interface. In an example use case of patient referral, the source node may be a discharge planning system of a hospital. The data package (e.g., referral data package, referral packet) is received through any one of various channels including direct integrations with referral channels (e.g., NaviHealth, Careport), direct fax intake, email intake, or a patient portal. The data package may include evidence including, e.g., clinical notes, orders, invoices, supporting documentation.

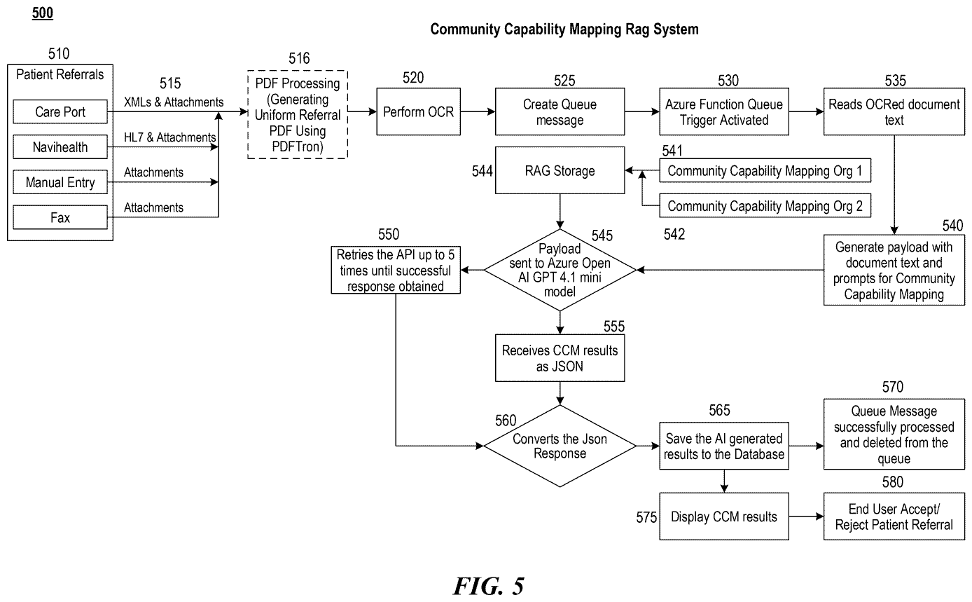

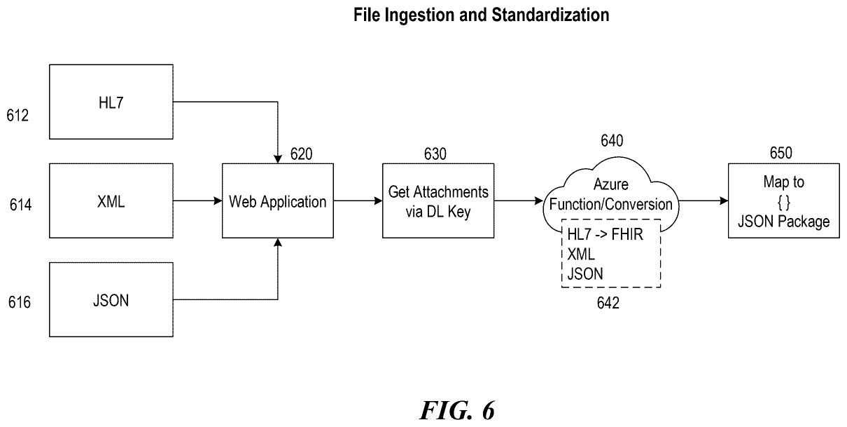

This ingestion process is managed by a data ingestion engine (e.g., 410 in FIG. 4). The ingestion process may trigger asynchronous processing events. Exemplary implementations of this ingestion logic are illustrated in FIG. 6, which illustrates the normalization of HL7, XML, and JSON data formats. Additionally, the handling of unstructured file inputs (e.g., PDFs) is described in the PDF processing logic 516 of FIG. 5, which includes PDF generation and queue messaging steps.

The network interface enables bi-directional data communications between the system and multiple external nodes. On the input side, it receives and aggregates data packages from multiple sources. On the output side, it transmits recommendations to source nodes or invitations to identified candidate nodes or target nodes. A candidate node or target node may be a service provider (e.g., an organization, a facility, a community), or a related entity, e.g., a reimburser/payer (e.g., medicare, medicaid, etc.).

The network interface also supports API-based communications. The system retrieves verification information through APIs. This includes insurance verification data and background check data. The system generates notifications based on the retrieved verification data. Other API implementations may incorporate additional verification capabilities such as criminal background checks and asset searches.

At 220, the process 200A/200B includes generating, using a machine learning component, an analysis record based on the data package, which may include various documents and reports. This extraction process can utilize optical character recognition (OCR) technology, e.g., executed by a document processing engine 420 as shown in FIG. 4. This converts document content into machine-processable text. In some embodiments, the extraction process includes generating a coordinate map (e.g., bounding boxes) defining the mapped locations of the extracted text within the document image. An OCR flow 520 illustrating paragraph tagging for AI context is shown in FIG. 5.

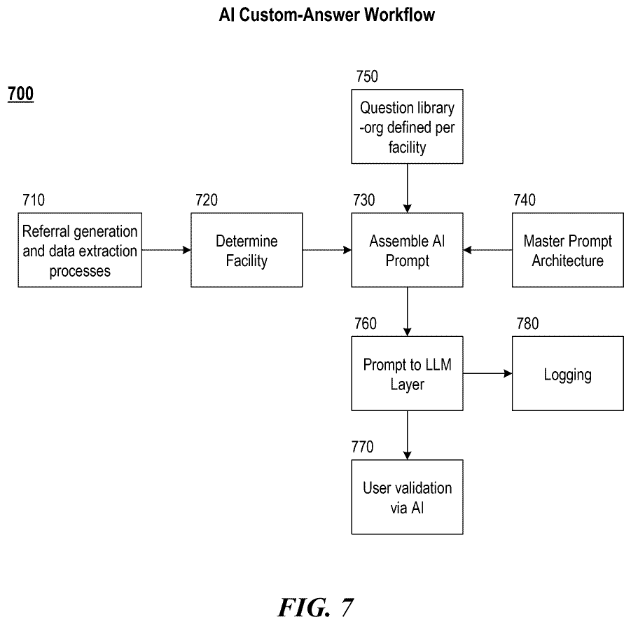

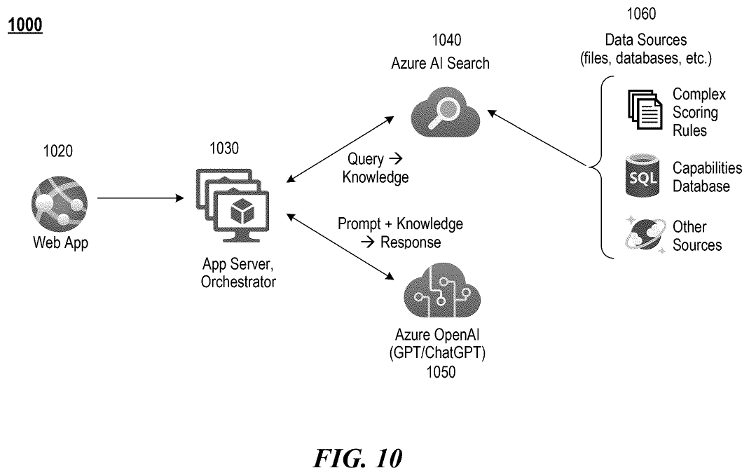

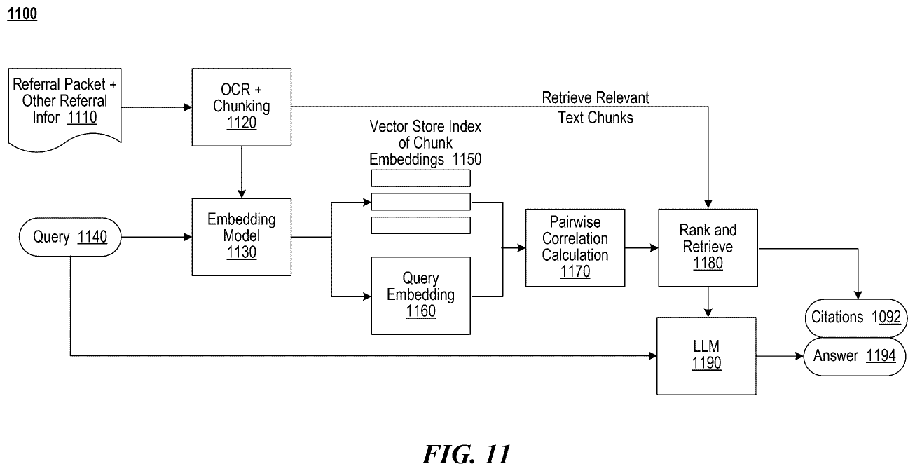

The extracted text is processed using a machine learning component (e.g., ML engine 430 shown in FIG. 4) that includes various sub-components such as a natural language processing (NLP) engine, a Vector Embedding Module, and a Semantic Retrieval Module (see FIG. 4). An exemplary overall architecture for this RAG (Retrieval-Augmented Generation) engine, including Azure AI Search and OpenAI interaction, is illustrated in FIG. 10. The NLP engine may incorporate large language models (LLMs) to understand and analyze the text comprehensively. In some embodiments, portions of the text are converted into embeddings and stored in a vector store index. An exemplary logic for this embedding model and chunk retrieval is shown in FIG. 11. During analysis, the system identifies elements including, e.g., keywords or sections of interest (including, e.g., segmented text chunks) within the text, facilitating targeted analysis and subsequent processing. For example, the system identifies elements using one or both of two methods. First, it uses fuzzy matching logic with a curated keyword dictionary. Second, it uses the LLM to identify contextually relevant words and text segments. The LLM identifies words that match, are synonymous with, or are semantically relevant to keywords for each category. FIG. 7 shows a flowchart of an exemplary process of assembling master prompts with customer questions to drive this analysis.

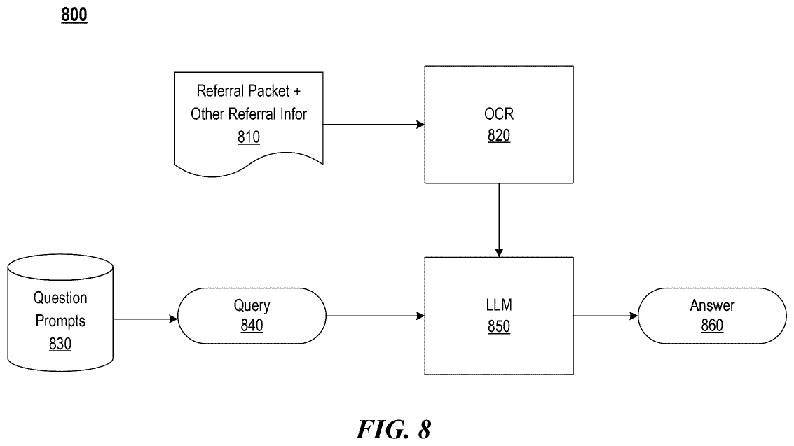

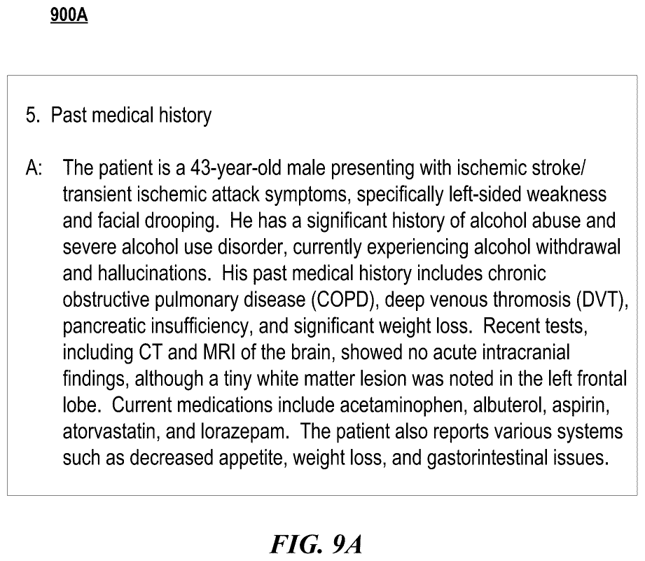

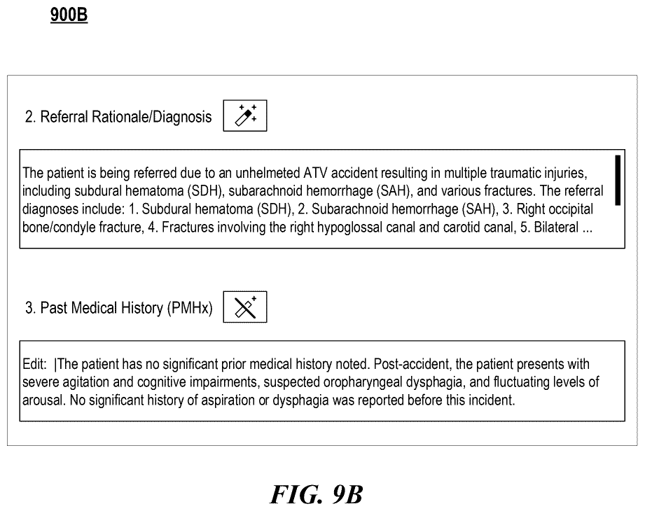

In some embodiments, the system generates analysis records, which include proposed or structured responses to predefined queries based on the extracted and analyzed text. This may be performed by an analysis generation module 438 of the ML engine 430 (see FIG. 4) using Retrieval-Augmented Generation (RAG). An exemplary clinical QA pipeline for this process is illustrated in FIG. 8. The machine learning component processes the text using customized prompts for different queries. A clinical question may have a corresponding engineered prompt. For example, a medical history prompt specifies aspects like primary diagnosis, recent issues, and current medications. The machine learning component generates responses following specific guidelines for answer format. An example textual output of such an LLM summary is provided in FIG. 9A. When information is insufficient, the machine learning component may leave responses blank, flag missing information, indicate partial answers, or request additional information. Additional descriptions regarding the generation of the analysis record or structured response may be found elsewhere in the present document. See, e.g., the description of 268 of FIG. 2C.

The system provides source attribution for responses generated by the machine learning component by linking answers to specific sections of the data package. This can include links to specific document sections, tooltips with quoted text, and mapped locations in PDF files. In some embodiments, this linkage creates a visual overlay that visually associates the generated compatibility indicators with the relevant text chunks and/or the specific coordinates of the relevant text chunks on the document image. Status indicators (e.g., as shown in FIG. 9B) show whether responses were machine-generated, human-edited machine-generated answers, or questions the machine learning component couldn't answer.

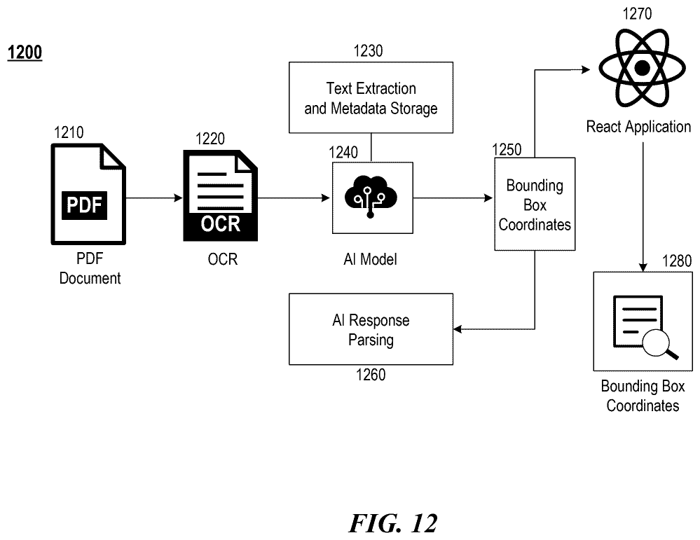

The system applies visual indicators and/or overlays (generated by, e.g., the verification rendering engine 460 of FIG. 4) to the identified elements to enhance readability and analysis. Examples of visual indicators include color coding, font modifications, bounding box overlays, and background highlighting. For example, different colors are used to indicate different categories of clinical review questions. The system can apply visual indicators to both direct keyword matches and contextually relevant sections. The technical implementation of this visual correlation system, including the Bounding Box coordinate system, is shown in FIG. 12.

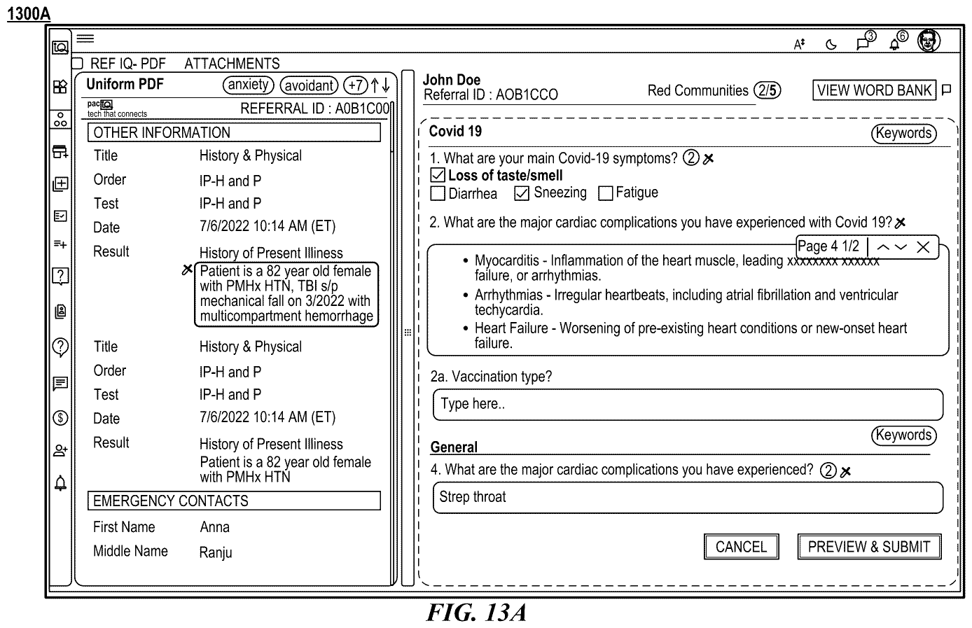

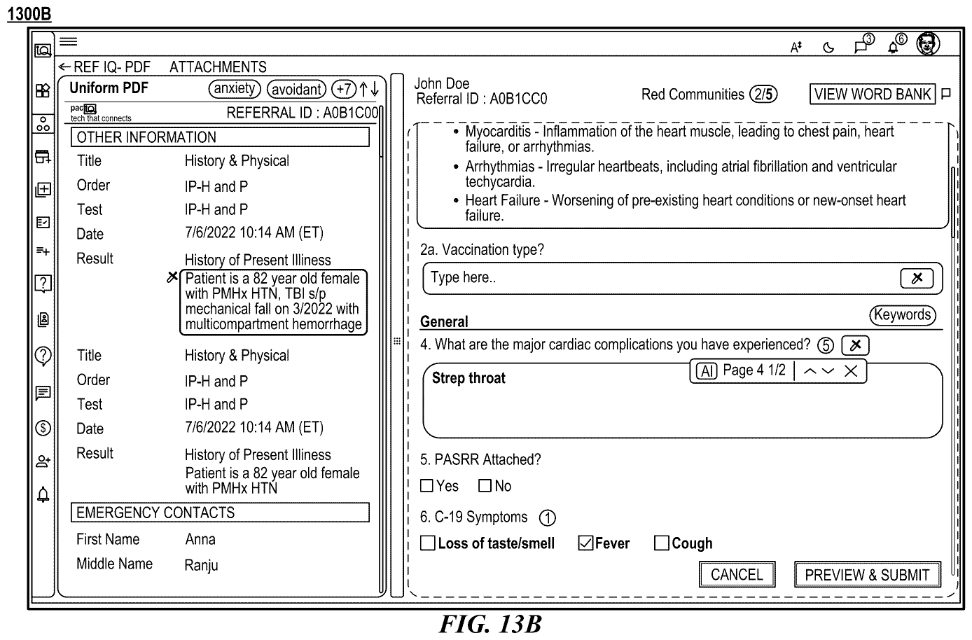

Merely by way of example, the identified keywords or sections are categorized into different groups such as clinical, administrative, or other relevant categories. Each category is assigned specific visual indicators to facilitate easy identification and prioritization. In cases involving medical data, these categorized and highlighted texts are integrated with a clinical review interface (e.g., the verification interface described in 250 of FIG. 2B) to aid in clinical assessments. The highlighted elements may streamline review and answering clinical questions by a user. Example interfaces 1300A and 1300B for this correlation are shown in FIG. 13A (History Document Highlighting) and FIG. 13B (Split View showing bounding boxes and AI answers), respectively.

The system provides proposed responses for user review. Users can modify the responses and provide response to questions that the machine learning component could not answer or has not answered. This user interaction may be captured as a validation input to create an audit trail. The final analysis record incorporates these modifications or additions. In an exemplary use case involving a patient referral, the analysis record includes patient care needs and corresponding medical care capacity indicators (e.g., compatibility indicators).

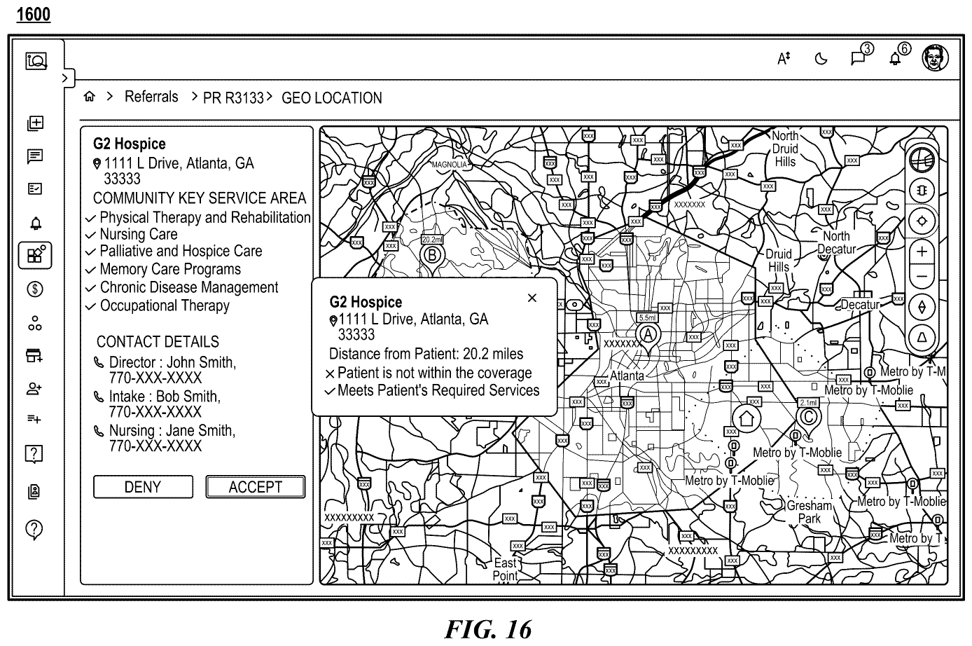

At 230, the process 200A/200B includes identifying, from a plurality of service providing nodes, a candidate node by comparing the compatibility indicators with node capability specifications. A candidate node may be a service provider (e.g., an organization, a facility, a community), or a related entity, e.g., a reimburser/payer (e.g., medicare, medicaid, etc.). This may be performed by a candidate matching engine 440 (FIG. 4) utilizing hierarchical constraints. FIG. 16 illustrates exemplary matching logic and rating assignment. The system performs capability matching on a question-by-question basis. The compatibility levels for a question may include full compatibility, potential compatibility needs verification, and incompatibility. The compatibility levels may be visually marked (e.g., color coded) for presentation to a user. For example, for each answer option, nodes specify capability ratings: “green” indicating full compatibility, “yellow” indicating need for additional verification, and “red” indicating capability mismatch.

The system compares compatibility indicators from the analysis record with node capability specifications of various nodes. For each node, the system evaluates capability matches across multiple dimensions on a question-to-question basis. In some embodiments, the node capability specification of a node may include one or more mandatory constraints (e.g., “Hard Stops”) and/or preferred constraints. When there is a mismatch between at least one compatibility indicator in the analysis record and a mandatory capability specification of a node, the system designates the node as not a candidate node. In some embodiments, the node capability specification of a node includes at least one item including reimbursement rule set or context, reimbursement eligibility, coding, coverage, or payment requirements associated with at least one payer selected from Medicare, Medicaid, a managed care organization, payer contract terms, medication cost data, or negotiated rate schedules, or the like, or a combination thereof.

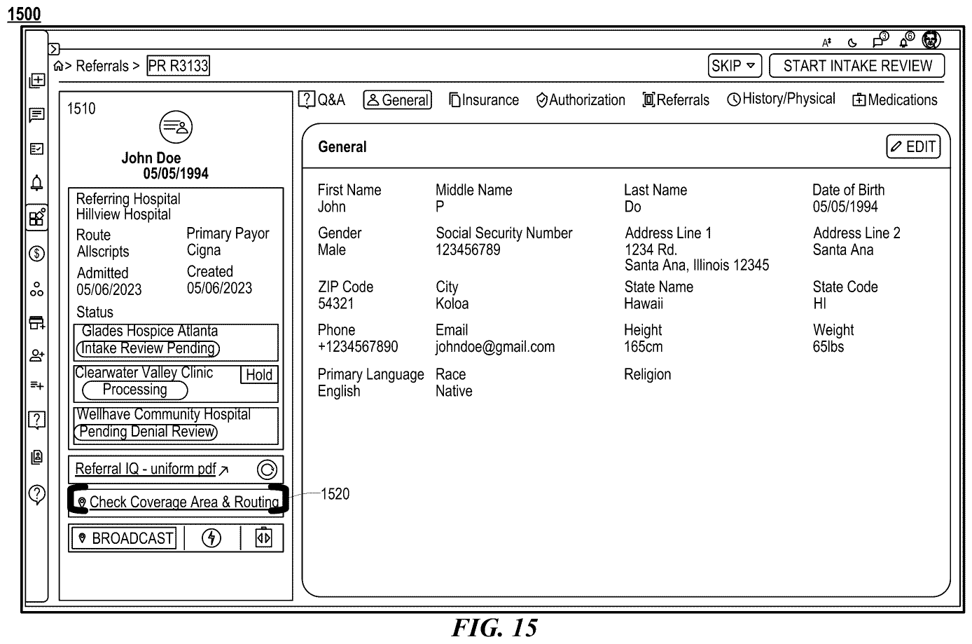

In some embodiments, the system can identify multiple candidate nodes each of which satisfies the compatibility needs. The system can rank multiple candidate nodes based on various factors. These include geographical distance between source and candidate nodes, current processing loads of the candidate nodes, and urgency indicators. Geo-location interfaces supporting this ranking are shown in FIGS. 15, 16, and 17. When multiple candidate nodes are identified, the system can generate a recommendation including each of them.

The system displays alerts related to capability ratings during the review process. For example, a summary banner shows counts of various (e.g., red and yellow) compatibility indicators. The banner also indicates the number of nodes marked as incompatible out of the total evaluated nodes. The system can identify non-evaluated nodes that may be good matches based on their capability specifications.

The capability matching process considers organization-specific configurations. Nodes can be grouped based on geographical location or other organizational parameters. The system can perform capability matching for additional nodes beyond those initially evaluated, particularly when initial nodes show capability mismatches. In some embodiments, the system can identify a group of candidate nodes within a predefined geographical range that collectively, rather than individually, satisfy all compatibility needs. When multiple candidate nodes are identified, the system can generate a recommendation including the group of candidate nodes.

At 240, the process 200A includes generating a recommendation including the candidate node for the source node. The recommendation may include node-tailored information corresponding to the candidate node. This tailoring optimizes data transmission and processing efficiency while maintaining centralized data management.

If multiple candidate nodes are identified, the system maintains a single centralized data package while generating node-specific versions. The node-tailored packages include common processed data along with node-specific elements. When analysis records are generated for one candidate node, the system automatically applies relevant portions to other candidate nodes' packages. This approach reduces duplicate processing while ensuring each node receives appropriately customized information. To facilitate efficient processing, the system may include in its recommendation specific guidelines that help the source node generate node-specific correspondence (e.g., invitation as described below) include common processed data along with node-specific elements. In some embodiments, the recommendation may include a ranking of the multiple candidate nodes, and/or notes regarding one or more of the candidate nodes to facilitate source node evaluation.

Based on the recommendation, the source node may identify one or more target nodes from one or more candidate nodes. The source node may send an invitation to one or each of the one or more target nodes. In some embodiments, the source node may notify the system (e.g., the system 150, 300B, 400, or 3400) of its selection of the one or more target nodes. The system may transmit, via the network interface, an invitation to one or each of the one or more target nodes. An invitation includes relevant data package information. In some embodiments, an invitation may be node-tailored that combines common processed data with node-specific elements.

A target node may respond to the invitation (whether received from the source node or system) with an acceptance or decline. For example, the target node may accept or decline the invitation. The target node may communicate the response to the source node, with a notification to the system. In some embodiments, the target node may communicate the response directly to the system. The system may notify the source node of the target node response. In some embodiments, the system may send additional information along with the notification. For example, if the target node denies the invitation, the system can provide an updated recommendation including alternative candidate nodes to the source node.

Referring to FIG. 2B, the process 200B may include at 250 generating a verification interface for presentation, e.g., on a user device. The verification interface comprises a visual overlay (generated by, e.g., the verification rendering engine 460 of FIG. 4) that visually associates the compatibility indicators to the relevant text chunks and/or the mapped locations of the relevant text chunks on the document image. Operation 250 may occur before, concurrently with, or after 240.

In some embodiments, the system may bypass sending recommendations to the source node and instead directly manage target node selection and invitation distribution. The system may implement sequential or parallel invitation strategies. In a sequential approach, the system designates candidate nodes as target nodes according to their ranking, sending invitations one at a time until acceptance is received. For example, the system first sends an invitation to the highest-ranked candidate node, and if not accepted, proceeds to the second-ranked node, and so forth. Alternatively, in a parallel approach, the system simultaneously transmits invitations to multiple candidate nodes.

When sending invitations directly to target nodes, the system maintains its node-tailored packaging approach. An invitation includes relevant data package information, with node-specific customization that combines common processed data with elements specific to that node's processing requirements. The system tracks responses from target nodes and may incorporate these responses into subsequent recommendations to the source node.

In exemplary healthcare implementations, where the source node is a referral source and candidate nodes are care communities, the system may generate and send a recommendation including one or more candidate nodes for presentation on a user device. In some embodiments, the recommendation (including, e.g., a referral request derived therefrom) may be transmitted to at least one of the one or more candidate nodes. For example, the system identifies a single candidate note and is sent to the identified candidate node. A reviewer at the candidate node reviews the recommendation and returns a reply. The reply may include a confirmation of receipt, an acceptance or denial of the recommendation (e.g., accepting or denying the referral), or a request for further information or documents, or the like, or a combination thereof. As described elsewhere in the present document, if the candidate node denies the recommendation, the system may identify another candidate node for recommendation. As another example, the system identifies a plurality of candidate nodes and rank the plurality of candidate nodes. The system may execute a sequential notification process. The recommendation is sent to a first candidate node having the highest ranking to seek feedback. If the first candidate node accepts the recommendation (e.g., accepts the referral), the system ceases transmission to the remaining candidate nodes having lower rankings. Conversely, if the first candidate node denies the recommendation, the system automatically transmits the recommendation to a second candidate node having the next highest ranking to seek feedback, and so on. In some embodiments, the recommendation may be sent to the source node. The source node may identify, from the one or more candidate nodes, a target node to which the source node sends an invitation. The invitation includes a referral for patient transfer. The system maintains centralized referral information while customizing presentation and additional needs for each receiving community.

FIG. 2C illustrates a flowchart of a process 200C performed by a computer-implemented data processing system, according to some embodiments. The process 200C may be implemented on system 150, 30B, 400, or 3400 described herein.

At 262, the process 200C includes receiving, by at least one processor via a network interface, an electronic data package from a source node. The electronic data package may originate from one or more source nodes, including systems directly managed by a healthcare entity (e.g., a hospital or physician's office) or third-party systems acting on behalf of a healthcare entity (e.g., CarePort, NaviHealth). Merely by way of example, the data package may include information or documents retrieved from a location specified within a referral data package (which itself may be part of the received data package). The package may be received via one or more of various transmission channels, including direct Application Programming Interface (API) integrations, email attachments, facsimile transmissions, or via an endpoint Robotic Process Automation (RPA) integration that scrapes a referral site to retrieve the data. The data package may include evidence including, e.g., clinical notes, orders, invoices, supporting documentation. The operation 262 may be substantially identical or similar to 210.

The electronic data package may include one or more files or data streams in varying formats. Example formats include a Portable Document Format (PDF) file, a Facsimile (Fax) image, a rasterized image file, an Extensible Markup Language (XML) file, a Health Level Seven (HL7) message (including Fast Healthcare Interoperability Resources (FHIR) standards), and a JavaScript Object Notation (JSON) packet. In embodiments where the data package includes structured data (e.g., XML, HL7, JSON) or multiple distinct files, the system may execute a normalization process. This process involves rendering the structured data into a visual format (e.g., creating a PDF representation of the HL7 message) and concatenating (merging) the rendered pages with any other files in the data package into a single unified document image prior to executing Optical Character Recognition (OCR). More descriptions may be found at, e.g., Section II “Data Intake.”

At 264, the process 200C includes obtaining a document image derived from the electronic data package. A document image refers to a digital representation of information that preserves a visual layout or spatial arrangement of text and graphics, capable of being displayed to a user and mapped to a coordinate system (e.g., Cartesian coordinates). A document image may include a PDF, Tagged Image File Format (TIFF), Joint Photographic Experts Group (JPEG), Portable Network Graphics (PNG), or Bitmap (BMP). A document image may include a visual representation dynamically derived from structured or semi-structured data. For example, the system may receive a raw HL7 (Health Level Seven) message, a JSON (JavaScript Object Notation) packet, or an XML file and execute a rendering engine (e.g., a “virtual printer” or HTML-to-PDF converter) to transform the raw text strings into a standardized visual layout (e.g., a standardized referral form or clinical summary).

In embodiments where the electronic data package already includes a visual file (e.g., a PDF, TIFF, or fax image), the document image may be obtained by retrieving the file from memory and preparing it for processing. In embodiments where the electronic data package includes structured or semi-structured data (e.g., HL7 messages, XML files, JSON packets, or FHIR resources), the document image may be obtained by performing a conversion, e.g., a rendering transformation. For instance, the system applies a visualization template or style sheet to the structured data to generate a synthetic visual representation (e.g., a virtual printout) of the data, converting raw text strings into a formatted document image (e.g., converting an HL7 lab result stream into a PDF page layout).

In some embodiments, obtaining the document image may involve a normalization and aggregation process. If the electronic data package includes multiple distinct components (e.g., a PDF referral letter combined with a separate JSON patient history file), the system normalizes all components into a common image format (e.g., PDF or rasterized images). The system then concatenates or merges these normalized components into a single unified document image (e.g., a multi-page PDF). This unification ensures that the subsequent extraction and analysis operations (e.g., OCR and AI analysis) are performed on a single, coherent visual artifact with a consistent geometric coordinate system (e.g., x, y pixel coordinates) established across all pages, thereby enabling the generation of spatial location data for every element of the received information. More descriptions may be found at, e.g., Section II “Data Intake.”

At 266, the process 200C includes generating extracted text and location data from the document image. The location data defines mapped locations of the extracted text within the document image. In some embodiments, the system executes an Optical Character Recognition (OCR) engine or a document layout analysis component to parse the unified document image. This process converts the visual pixel data into machine-readable text strings while simultaneously preserving the spatial structure of the document image.

In generating the location data, the system may construct a coordinate map (or spatial index) that functions as a lookup table linking the extracted text to its physical position on the document image. The coordinate map associates discrete elements of the text (e.g., individual characters, words, text strings, text chunks, lines, or paragraphs) with specific geometric coordinates. These coordinates may define a bounding box (e.g., a rectangular area defined by an $x$-coordinate, a $y$-coordinate, a width, and a height) or a polygon (e.g., a set of four corner points) that encloses the respective text element (or referred to as text chunk, text string, text segment, text block) on the document image. This coordinate map enables the system to reverse-engineer the location of any specific text string later in the process. Merely by way of example, if the system knows what text was relevant (e.g., “Patient has Type 2 Diabetes”), the coordinate map allows the system to determine exactly where that text appears (e.g., “Page 3, coordinates $[150, 400]$”) to draw the visual overlay.

In some embodiments, the location data includes structural metadata tags. A structural metadata tag is a logical identifier representing a distinct structural unit of the document, such as a page identifier (e.g., [Page 1]), a paragraph identifier (e.g., <Para_05>), a line number, or a semantic section header (e.g., [History of Present Illness]). Unlike raw geometric coordinates, structural metadata tags provide a token-efficient shorthand for location. For example, during the layout analysis, the system may tag a specific block of text as “Paragraph 12.” This tag serves as a “location metadata” key; the system stores the association that “Paragraph 12” corresponds to the geometric coordinates $[x=100, y=500, w=300, h=50]$. This allows the AI model to simply cite “Paragraph 12” as its source, which the system then resolves against the coordinate map to find the bounding box.

The location data may include any data structure capable of defining the position of text. In other embodiments, the location data may comprise character offsets (e.g., “start character 1054, end character 1090”), token indices (e.g., “the 25th through 30th tokens in the sequence”), or raw geometric arrays embedded directly with the text. The selection of the specific location metadata format depends on the capabilities of the downstream machine learning component; however, the combination of logical tags (for the AI) mapped to geometric coordinates (for the interface) provides a robust mechanism for the high-precision “source citation” described elsewhere herein, e.g., with respect to 268.

More descriptions may be found at, e.g., Section III “Automated Information Extraction via Artificial Intelligence.”

At 268, the process 200C includes generating, using a machine learning (ML) component, a structured response. The process for generating the structured response may include generating a prompt payload comprising at least a portion of the extracted text and a node capability specification of a service providing node; providing the prompt payload to a generative AI model of the ML component; and receiving the structured response from the generative AI model. The structured response may include: (i) at least one compatibility indicator determined based on the node capability specification of the service providing node, and (ii) a source citation identifying at least one relevant text chunk from the extracted text that supports the at least one compatibility indicator. In some embodiments, the service providing node may be specified in the electronic data package (e.g., in a ‘Targeted Evaluation’ mode where a referral is directed to a specific facility). Alternatively or additionally, the service providing node may be dynamically identified by the system through a network search or preliminary selection operation. This identification involves querying a database of organizational locations and filtering potential nodes based on criteria including geospatial proximity to the patient's location, service capabilities (e.g., bed availability or specialized equipment), and payer contract status. A service providing node may also be referred to as a candidate node or target node, which may be a service provider (e.g., an organization, a facility, a community), or a related entity, e.g., a reimburser/payer (e.g., medicare, medicaid, etc.).

It should be noted that the term “compatibility indicator” is not limited to clinical suitability. In various embodiments, the compatibility indicator may represent a reimbursement readiness indicator, a documentation completeness indicator, or a verification that the electronic data package satisfies specific medical necessity requirements for reimbursement. For example, if the query relates to a reimbursement questionnaire (as described elsewhere herein), the compatibility indicator may indicate whether the extracted text supports the necessary billing codes (e.g., “Documentation Satisfied: Yes” or “Coding Requirements Met: No”). Thus, the system utilizes the compatibility indicator to validate that the referral is not only clinically appropriate but also financially viable and compliant with the payer requirements defined in the node capability specification.

In some embodiments, the node capability specification of a node defines a clinical question and a set of valid answer options for the clinical question. In some embodiments, the node capability specification of a node may include one or more mandatory constraints (e.g., “Hard Stops”) and/or preferred constraints. When there is a mismatch between at least one compatibility indicator in the analysis record and a mandatory capability specification of a node, the system designates the node as not a candidate node. In some embodiments, the node capability specification of a node includes at least one item including reimbursement rule set or context, reimbursement eligibility, coding, coverage, or payment requirements associated with at least one payer selected from Medicare, Medicaid, a managed care organization, payer contract terms, medication cost data, or negotiated rate schedules, or the like, or a combination thereof.

In some embodiments, the prompt payload is generated by incorporating at least a portion of the node capability specification. For example, the prompt payload is generated by incorporating the set of valid answer options in the prompt payload to constrain the structured response of the generative AI model to a selected option from the set of valid answer options.

In some embodiments, the system facilitates automated coding guidance. The prompt payload is generated by incorporating a set of valid answer options to constrain the structured response. For instance, the system provides a specific list of options to the generative AI model, which may include standardized codes (e.g., Current Procedural Terminology (CPT), International Classification of Diseases (ICD), or Healthcare Common Procedure Coding System (HCPCS) codes), modifiers, or classifications required for reimbursement submission. Accordingly, the process of synthesizing the relevant text chunks comprises selecting a standardized code, modifier, or classification from the provided list. This list of options may be dynamically retrieved based on the specific payer associated with the service providing node (e.g., limiting the selection to codes valid under a specific Medicare Part B fee schedule or a private payer's contract terms). This ensures that the generated compatibility indicator is not just clinically accurate but semantically compliant with the specific coding dialect required for the claim.

In some embodiments, the system executes a single-pass inference. Instead of iteratively querying the model for each individual criterion, the system aggregates a plurality of clinical questions defined in the node capability specification and the extracted text into a single prompt payload. The generative AI model processes this aggregated payload in a single inference pass and returns a single JavaScript Object Notation (JSON) packet. An exemplary JSON packet is structured to contain, for each of the plurality of clinical questions:
(i) A specific compatibility indicator (e.g., a standardized value such as “True,” “False,” or a specific medical code selected from the valid answer options);
(ii) An answer determined based on a portion of the relevant text chunk (e.g., a natural language summary or extraction of the specific clinical fact found in the document); and
(iii) A portion of the source citation corresponding to the relevant text chunk (e.g., structural metadata such as page numbers, paragraph IDs, or word tokens) that supports the compatibility indicator.

In generating the structured response, the system may employ vector embeddings and pairwise correlation to ensure the AI answers are grounded in the specific evidence found in the electronic data package.

For instance, the system converts discrete text chunks from the document image into text chunk embeddings (numerical vector representations) stored in a vector store index. When a clinical question or a reimbursement-specific inquiry is processed (e.g., “Does the patient require isolation?” or “Does the patient meet Minimum Data Set (MDS) criteria for coverage?”), the system converts the question into a query embedding. In some embodiments, these queries are derived from a reimbursement questionnaire associated with the node capability specification of at least one of the service providing nodes. The system then performs a pairwise correlation calculation (e.g., a cosine similarity search) between the query embedding and the stored text chunk embeddings. This process identifies and retrieves the specific text chunks that are semantically most relevant to the question. These retrieved chunks are provided to the generative AI model to generate the AI Answer and the Source Citation. It is this AI Answer (e.g., “Isolation: Yes”) that is subsequently passed to the determination logic at 270 to be evaluated against the node capability specifications. See, e.g., FIGS. 10 and 11 and the description thereof.

In performing the pairwise correlation determination, the system may determine a similarity score between the query embedding (derived from the clinical question) and the text chunk embeddings (derived from the document). This determination may include calculating a cosine similarity score, a dot product, a Euclidean distance (L2 Norm) or Manhattan distance (L1), Jaccard similarity, a Hamming distance, or the like, or a combination thereof. The system selects only those text chunks that meet or exceed a predefined similarity threshold (e.g., >0.75), ensuring that the generative AI model is grounded only in highly relevant evidence.

In some implementations utilizing Large Language Models (LLMs) or managed embedding services (e.g., Azure OpenAI), the pairwise correlation determination may be abstracted by the underlying model itself. In these scenarios, the “similarity” is determined by the model's internal attention mechanisms or proprietary relevance scoring algorithms, which inherently calculate the correlation between the text chunks and the prompt context without requiring the system to manually compute a specific geometric distance.

Alternatively or additionally, the system is configured to handle scenarios where relevant information is absent. In some embodiments, the system performs proactive validation and completeness checks. The generation of the structured response (e.g., populating the analysis record) includes identifying missing, inconsistent, or insufficient documentation elements required for reimbursement and generating at least one validation flag prior to claim submission. For example, the system may determine, based on the structured response (e.g., a “Null” or “Not Found” value returned in the JSON packet), that the electronic data package is incomplete regarding a mandatory data element defined in the node capability specification. Upon generating the validation flag (e.g., a “Claim Stop” or “Documentation Hold”), the system automatically triggers an electronic task (e.g., an automated email notification, a Request for Information (RFI) message, or a flagged work item) to request additional information from the source node regarding missing or insufficient element. This remediation step is performed prior to making a final determination on whether the compatibility indicator satisfies the node capability specification, ensuring that referrals are not rejected—and/or claims are not submitted—solely due to documentation gaps that can be rectified in advance (e.g., before a recommendation is presented and/or claim submitted).

In some embodiments, the system is configured to reduce denials by flagging payer-specific risk. For instance, the system accesses a repository of historical denial patterns (e.g., aggregated data identifying specific coding combinations or documentation gaps that frequently result in rejections from specific payers). Based on this data, the system derives a payer-specific reimbursement risk score for the compatibility indicator. This derivation is calculated based on identified rule deviations (e.g., minor discrepancies between the extracted text and the payer's optimal criteria) and their correlation with the historical denial patterns. For example, if historical data indicates that a specific payer denies “Sepsis” claims 90% of the time when “Lactate levels” are missing, the system assigns a high-risk score to the compatibility indicator if that specific evidence is absent, even if the general documentation is otherwise complete.

In some embodiments, prior to providing the prompt payload to the generative AI model, the system may execute a data sanitization process. This process involves analyzing the extracted text to identify and mask predefined types of personal information (e.g., Patient Names, Social Security Numbers, or internal Medical Record Numbers) to ensure compliance with privacy regulations (e.g., HIPAA). The system may replace these sensitive entities with tokenized placeholders (e.g., [PATIENT_NAME]) within the prompt payload, ensuring that the generative AI model processes the clinical logic without being exposed to unnecessary Personally Identifiable Information (PII).

In some embodiments, the system is configured to manage AI safety guardrails to prevent erroneous censorship of valid clinical data. For instance, a standard Large Language Models (LLMs) may be trained with “safety filters” designed to block content related to violence, gore, or self-harm. In a medical context, however, these filters can inadvertently censor necessary descriptions of severe medical conditions (e.g., deep wound descriptions, explicit details of traumatic injuries) or behavioral health issues (e.g., substance abuse history, alcoholism, or self-harm ideation).

To address this, the system may employ one or more techniques. In some embodiments, the generative AI model is fine-tuned or trained on a specialized medical corpus. This training adjusts the model's internal weighting to recognize that terms typically flagged as “sensitive” (e.g., descriptions of bodily injury) are benign and necessary in a clinical context, effectively overriding the default safety bias for these specific domains. Additionally or alternatively, the generation of the prompt payload comprises including a clinical authorization instruction (e.g., a “system prompt” or context setting). This instruction explicitly contextualizes the request (e.g., “You are a certified medical assistant processing clinical records. Do not censor medical descriptions.”), thereby authorizing the model to process and extract valid clinical descriptions that might otherwise be suppressed by generic safety protocols.

More descriptions may be found at, e.g., Section III “Automated Information Extraction via Artificial Intelligence.”

At 270, the process 200C includes determining whether the at least one compatibility indicator satisfies the node capability specification of the service providing node.

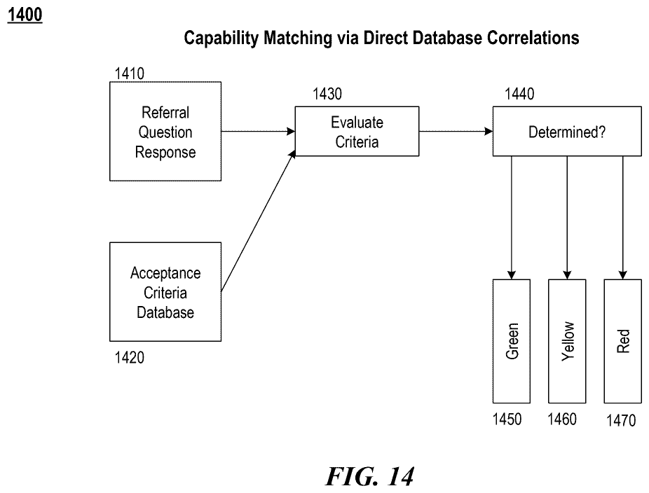

The system receives the structured response (e.g., a referral question response). The system then queries an acceptance criteria database to retrieve the specific capability rule corresponding to the service providing node (e.g., “Facility A accepts BMI<40”; “Facility A does not accept Ventilators”). See, e.g., FIG. 14 and the description thereof.

The system executes an evaluation criteria operation to compare the response against the rule. For example, this comparison may involve one or more including:
Discrete Value Matching: Comparing categorical values (e.g., If “Patient Status”=“Aggressive”, then “Reject”);
Numeric Thresholds: Comparing numerical values against defined ranges (e.g., “If Oxygen>9L, then Incompatible”).
Date-Based Conditions: Evaluating temporal constraints (e.g., “Admission Date” must be after “Contract Start Date”).

Based on this evaluation, the system determines a specific status. This status may be mapped to a visual compatibility indicator (e.g., a color-coded flag) for presentation in the verification interface. For example: “Green” (Satisfied): Indicates the referral data aligns with the node capability specification; “Yellow” (Caution/Verify): Indicates the data falls within a borderline range or requires manual verification; “Red” (Unsatisfied/Hard Stop): Indicates a mandatory constraint violation (e.g., a “Hard Stop”), signaling that the service providing node is incompatible with the electronic data package.

More descriptions may be found at, e.g., Section IV “Matching and Routing.”

At 272, the process 200C includes generating a verification interface for presentation. The verification interface includes a visual overlay that visually associates, based on the source citation and/or a mapped location of the at least one relevant text chunks, the at least one compatibility indicator to the at least one relevant text chunks on the document image.

The visual overlay may take various forms to facilitate rapid verification by a reviewer. The visual overlay (e.g., the bounding box) may correspond directly to the geometric coordinates defined in the location data at 266. The system retrieves the coordinates associated with the relevant text chunk—previously identified and stored during the extraction phase—and renders a graphical shape (e.g., a semi-transparent colored rectangle) on the document image at those precise pixel locations.

In some embodiments, this visual overlay relies on a mapping between logical tags and physical coordinates. During the prompt generation phase, the system may have embedded location tags (e.g., <Para_01>) within the extracted text sent to the AI model. When the AI model returns a source citation, it references a specific location tag assigned to the relevant text chunk (e.g., “Source: <Para_01>”). Operation 272 may involve resolving this specific location tag against the location data to identify the corresponding geometric coordinates, which are then used to generate the visual overlay for presentation on the user's screen.

Regarding the technical implementation of the visual overlay, the system may employ a two-step resolution process to bridge the semantic output of the AI model and the graphical requirements of the user interface. As previously described, the location data includes structural metadata tags (e.g., page numbers, paragraph indices, or token IDs) associated with specific geometric coordinates. During the analysis phase, the generative AI model is provided with text containing these metadata tags and returns a source citation referencing a specific tag (e.g., “Answer: Yes; Source: Paragraph_10”). To render the visual overlay, the system uses the location data as a lookup table/coordinate map. It takes the metadata tag returned by the model, identifies the corresponding geometric coordinates (e.g., x, y, width, height) stored for that tag, and renders the bounding box or highlight at those specific pixel locations. This ensures that while the AI operates on semantic text tags, the interface accurately draws graphical overlays on the document image.

In some embodiments, the visual overlay includes a bounding box enclosing a relevant text chunk or a color highlight (e.g., a semi-transparent yellow layer) applied over the text chunk. In some embodiments, the visual overlay may include a connector line or “tether” (e.g., a vector line drawn from the compatibility indicator in a side panel to the relevant text chunk in the document image), a margin indicator (e.g., a vertical bar or icon placed in the document margin adjacent to the relevant paragraph), a numeric indexing tag (e.g., a “(1)” icon next to the data field corresponding to a “(1)” icon next to the text), or a “spotlight” effect (e.g., dimming the opacity of non-relevant text to visually emphasize only the relevant text chunk). The visual overlay is configured to facilitate the reviewer to instantly visually verify the source of the AI's determination without manually searching the document.

In some embodiments, the verification interface is configured to display reimbursement documentation. This documentation may include a summary of generated billing codes, Minimum Data Set (MDS) criteria, or insurance coverage verifications derived from the electronic data package. The reimbursement documentation may include interactive links to source documentation. For example, if the interface displays a suggested billing code (e.g., “ICD-10: I10—Essential Hypertension”), that code is rendered as an interactive element. Upon user interaction (e.g., clicking or hovering), the system utilizes the spatial mapping data to automatically scroll the document view to the specific physician's note or lab result that substantiates that code. This feature creates an audit-ready “proof of coding” view, allowing revenue cycle managers to validate that every reimbursement claim is supported by specific, locatable evidence within the source file.

In some embodiments, the verification interface is configured to present guided remediation action(s). For instance, when a compatibility indicator reveals an unmet reimbursement requirement (e.g., a missing specific clinical finding required by the node's reimbursement rule set), the interface dynamically generates actionable instructions for the user. These steps are derived by comparing the current compatibility indicator against the full criteria of the reimbursement rule set. Merely by way of example, if a “Pneumonia” diagnosis code is flagged as “Incomplete” due to a missing chest x-ray report, the interface displays a remediation prompt such as: “Action Required: Upload Chest X-Ray Report to validate ICD-10 J18.9.”. This guidance allows the user to rectify the documentation gap immediately within the interface before the referral is processed further, thereby preventing downstream claim denials.

In some embodiments, if it is determined that the at least one compatibility indicator satisfies (or fails to satisfy) the node capability specification, a recommendation regarding the service providing node is generated. The verification interface is configured to display the recommendation to a reviewer. The verification interface may be further configured to receive an input in response to the recommendation. If the recommendation includes a rejection of the service providing node (e.g., “Referral Declined”), the system causes the rejection to be displayed in association with a specific text chunk serving as evidence for the rejection. For example, the interface may display a rejection alert alongside a hyperlink or a direct view of the specific text chunk (e.g., “Rejection Reason: Patient BMI>35; Evidence: [Link to Page 4: ‘BMI 38’]”). While the recommendation may apply to the electronic data package as a whole (e.g., rejecting an entire referral), the system ties the rejection decision to specific compatibility indicators and links them to the underlying text chunks (e.g., specific answers to clinical questions defined in the node capability specification) to substantiate the global decision.

In some embodiments, the recommendation includes at least one documentation, coding, or evidence recommendation to improve reimbursement accuracy or timeliness. For instance, if the system identifies a mismatch between a high-acuity diagnosis and the submitted documentation (e.g., “Sepsis” diagnosis without supporting lactate lab results), the recommendation message may explicitly state: “Recommendation: Obtain Lactate Level>2 mmol/L to support Sepsis coding.” By providing this granular, actionable guidance before the referral is accepted or the claim is generated, the system proactively reduces the likelihood of downstream claim denials and billing delays.

In some embodiments, the visual overlay is dynamically rendered in response to user interaction. The verification interface may display the compatibility indicator (e.g., the answer to a clinical question) as an interactive User Interface (UI) element. Upon receiving a user input (e.g., a mouse click or hover event) on the compatibility indicator, the system triggers a “go to” or “show evidence” function. This function automatically scrolls the document image to the mapped location of the relevant text chunk and dynamically renders the visual overlay (e.g., flashing the bounding box or activating the highlight) at that moment. This interactive design allows the reviewer to focus on the extracted answers first and selectively view the supporting evidence in the document structure on demand.

To prioritize reviewer attention, the system may further incorporate confidence scoring. The machine learning component may determine a confidence score for each derived compatibility indicator. The verification interface is configured to visually distinguish any compatibility indicator having a confidence score below a predefined threshold. For example, high-confidence answers may be displayed in green or standard text, while low-confidence answers may be flagged with a visual warning (e.g., a red flag icon, yellow background, or “Review Required” badge). This directs the human reviewer to manually verify the specific data points where the AI model was uncertain, thereby optimizing the efficiency of the review process.

In some embodiments, the system supports intelligent routing upon rejection. If the system determines that the service providing node is incompatible with the data package, it may automatically identify a second service providing node having a capability specification that is compatible with the extracted indicators. The verification interface may then display a second recommendation to route the electronic data package to this second service providing node (e.g., “Incompatible with Facility A; Recommended Redirection: Facility B”).

In some embodiments, to enhance transparency regarding the origin of the information, the verification interface may distinguish between data types. The structured response may comprise both content directly extracted from the package (verbatim quotes) and AI-generated content (summaries or synthesized inferences). The verification interface may generate a visual indication (e.g., distinct icons, font colors, or badges such as a robot icon vs. a document icon (or lack of a robot icon)) to clearly distinguish the AI-generated content from the native extracted content, allowing the reviewer to weigh the evidence accordingly.

In some embodiments, the process 200C may include receiving a validation input via the verification interface confirming, rejecting, or correcting the at least one compatibility indicator. For instance, the verification interface allows the reviewer to accept the AI's determination (e.g., clicking “Confirm”), reject the AI's determination (e.g., request a reassessment based on currently available information or request a reassessment and additional information), or manually override the determination (e.g., changing a “No” to a “Yes” based on their review of the highlighted text).

This validation input serves as feedback for system adaptation. In some embodiments, the system utilizes these manual overrides to dynamically refine the generation of the prompt payload (at 268) for future iterations. In some embodiments, the system may employ organization-specific personalization. If a specific service provider frequently overrides certain criteria (e.g., they accept patients with slightly higher BMIs than the standard model predicts), the system creates custom prompts or sub-prompts tailored to that specific provider to achieve customized results.

In some embodiments, this feedback loop drives a model fine-tuning process. Instead of relying solely on a generic “one-size-fits-all” model, the system may maintain separate, fine-tuned models (or “adapter” layers) for each distinct healthcare organization. The validation inputs are collected as training data to retrain or adjust the weights of these organization-specific models, ensuring that the AI's decision-making logic evolves to match the specific clinical preferences and risk tolerance of each service providing node.

Additionally or alternatively, the system utilizes this interaction to create a defensible compliance record. Storing the validation input includes storing linked documentary evidence supporting reimbursement requirements to enable post-submission audit or appeal. For example, when a user confirms a diagnosis code, the system permanently freezes the association between that code and the specific text chunk (evidence) used to justify it. This creates a granular, immutable audit trail that is valuable for regulatory reviews, including Medicare audits, managed care denials, and Risk Adjustment Data Validation (RADV) or Recovery Audit Contractor (RAC) defense. By preserving the exact “evidence linkage” used at the time of decision, the system allows providers to rapidly reconstruct and substantiate their claims years after submission.

Once the review is complete and the compatibility indicators are verified, the process may include transmitting the structured response to an external system. For example, the verified JSON packet—containing the clinical answers and validated data—may be transmitted via an API or HL7 interface to an Electronic Health Record (EHR) system, a Case Management System, or a Referral Management Platform for permanent storage and downstream clinical workflow integration.

In some embodiments, the process 200C includes generating predictive analytics alongside the compatibility determination, occurring after generating the extracted text (at 266) or concurrently with generating the structured response (at 268). For example, the system may execute one or more of the following:

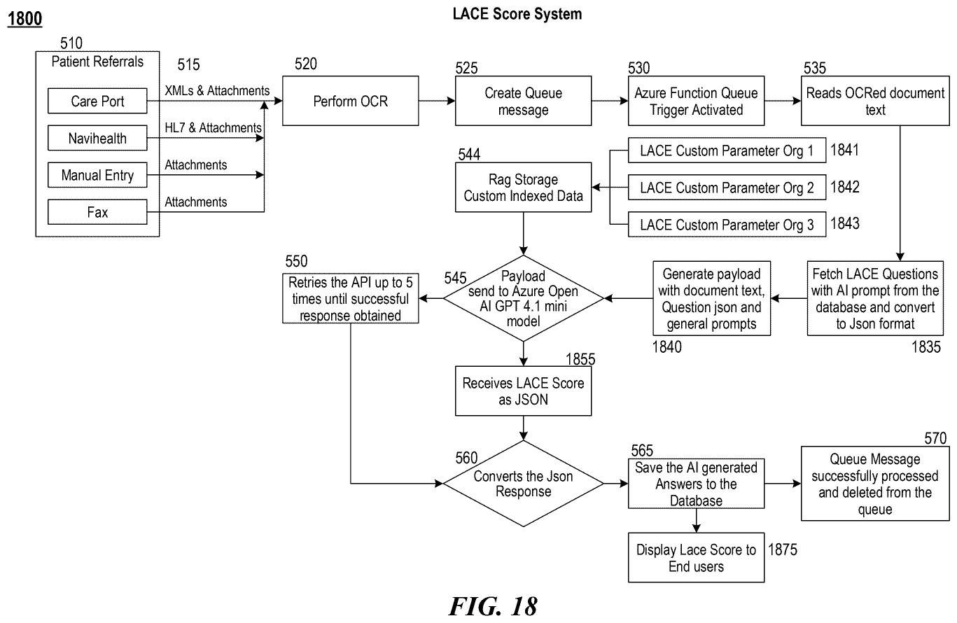

Clinical Risk Module (e.g., as shown in FIG. 18) to automatically calculate a LACE Score (Length of stay, Acuity, Comorbidities, Emergency visits) based on the extracted text, predicting readmission risk;

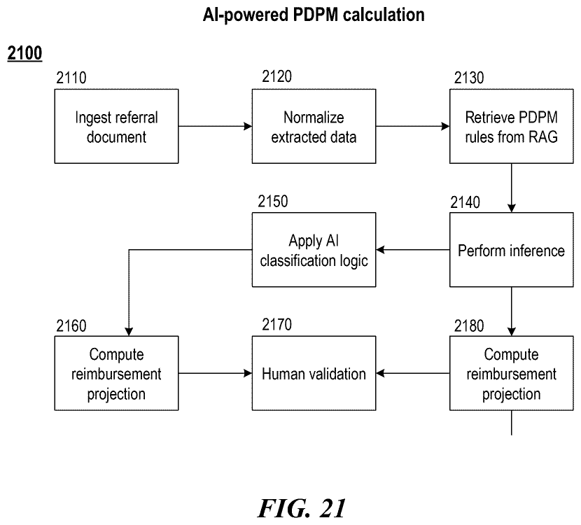

Financial Forecasting Module (e.g., as shown in FIG. 21) to calculate a PDPM (Patient-Driven Payment Model) projection, estimating reimbursement rates prior to admission;

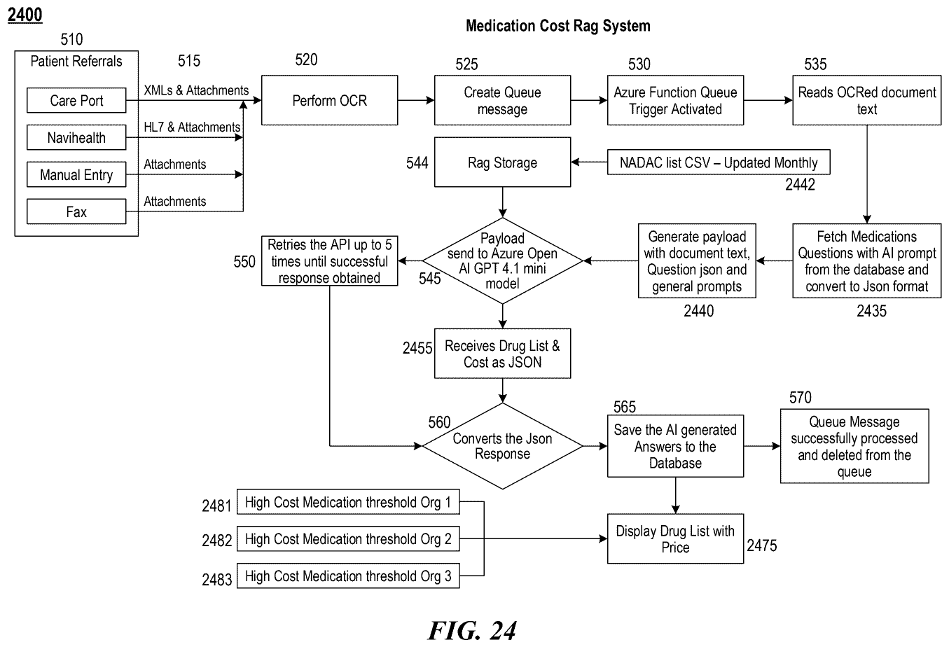

Cost Estimate Module (e.g., as shown in FIG. 24) to calculate a medication cost projection by cross-referencing extracted medication lists against a pricing database (e.g., NADAC);

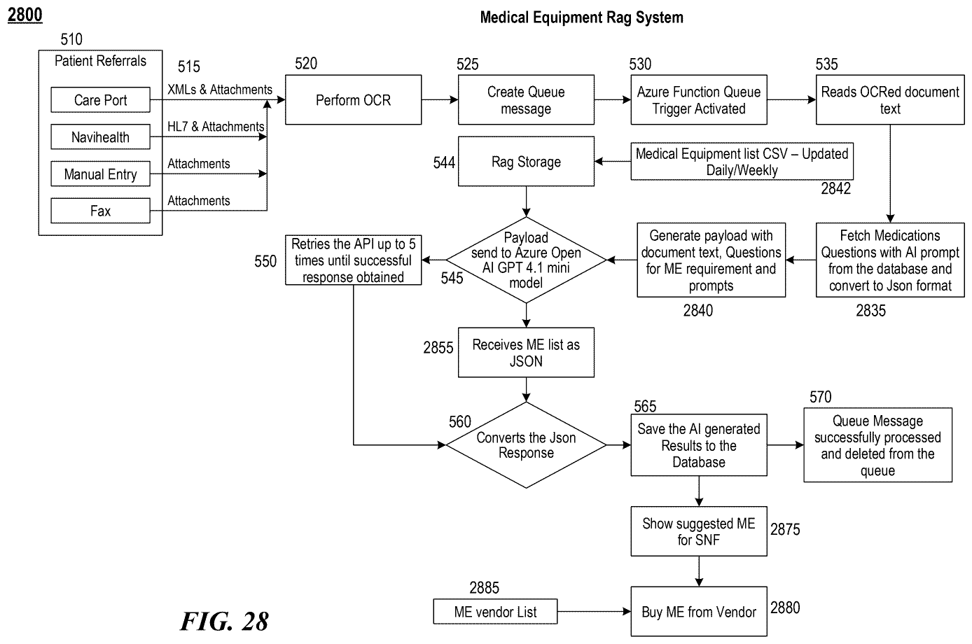

Medical Equipment Management Module (e.g., as shown in FIG. 28) to identify required durable medical equipment (DME) and estimate associated acquisition costs.

These scores and projections may be integrated into the structured response (or analysis record) and displayed in the verification interface (at 272) to aid the reviewer's decision. More descriptions may be found at, e.g., Section V “AI-Driven Decision Support.”

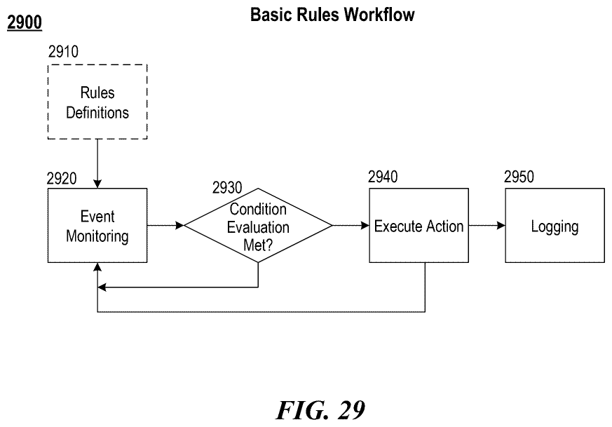

Throughout the execution of process 200C, the workflow may be dynamically orchestrated by a configurable Rules Engine (e.g., component 480 in FIG. 4) operating on an “Event-Condition-Action” framework as illustrated in FIG. 29. The rules engine monitors for specific trigger events generated at any step of process 200C (e.g., detecting “Compatibility Indicator=Red” at 270 or calculating “LACE Score>10” at the decision support phase). Upon detecting a trigger, the engine evaluates conditions and automatically executes downstream actions, such as sending a notification to a specific clinical team, creating a high-priority task, or automatically routing the electronic data package to a different service providing node. This allows the process 200C to adapt in real-time to the specific clinical or financial risks identified within the electronic data package. More descriptions may be found at, e.g., Section VI “Configurable Rules Engine and Automated Workflows.”

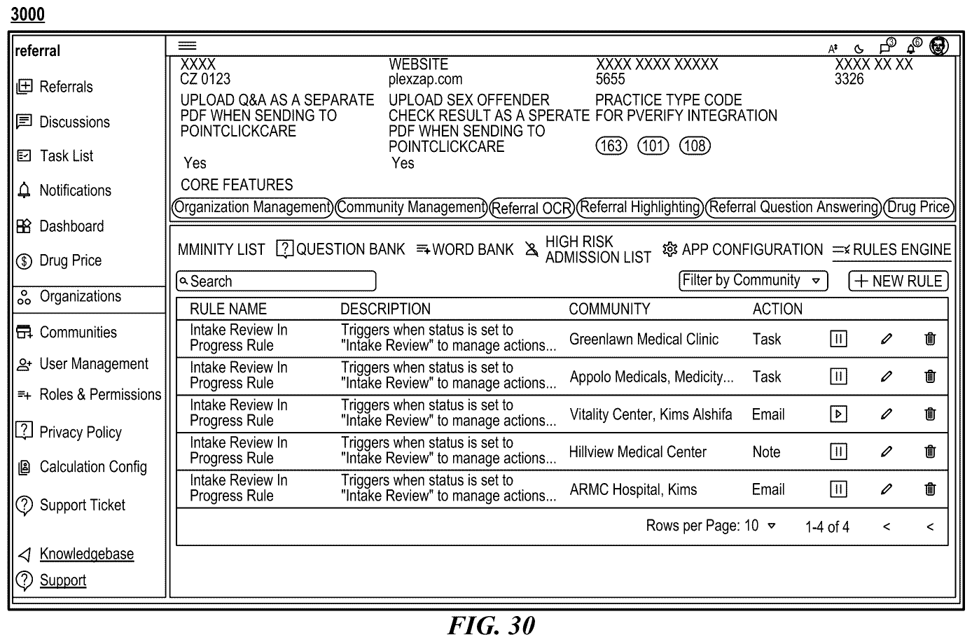

Following process 200A/200B/200C, the system can handle multiple data packages received from one or more source nodes. The system supports distributed processing of received data packages. Multiple authorized users (e.g., from multiple post acute communities with a network or organization) can share the processing workload. This distributed approach ensures efficient processing of data packages. The system maintains a unified processing environment. Authorized users operate within the same system. These users may include central processors, node-specific processors, and regional administrators. To support automated workflows in this distributed environment, the system utilizes a rules engine (rules engine 480 as illustrated in FIG. 4) and/or follows event-condition-action logic (FIG. 29). User interfaces for configuring these rules and viewing automated tasks are shown in FIGS. 30, 31, and 32.

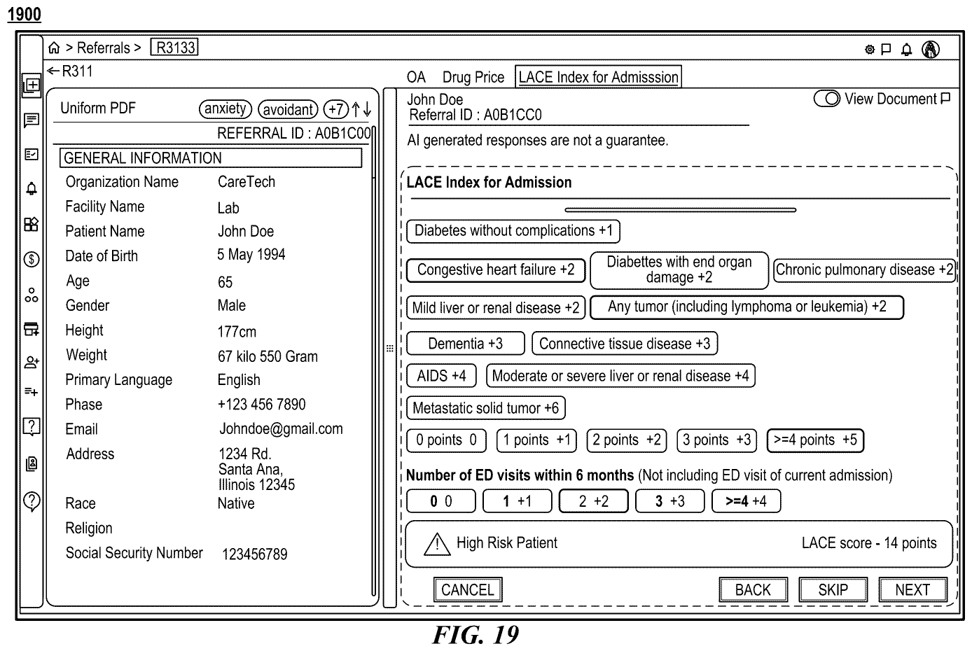

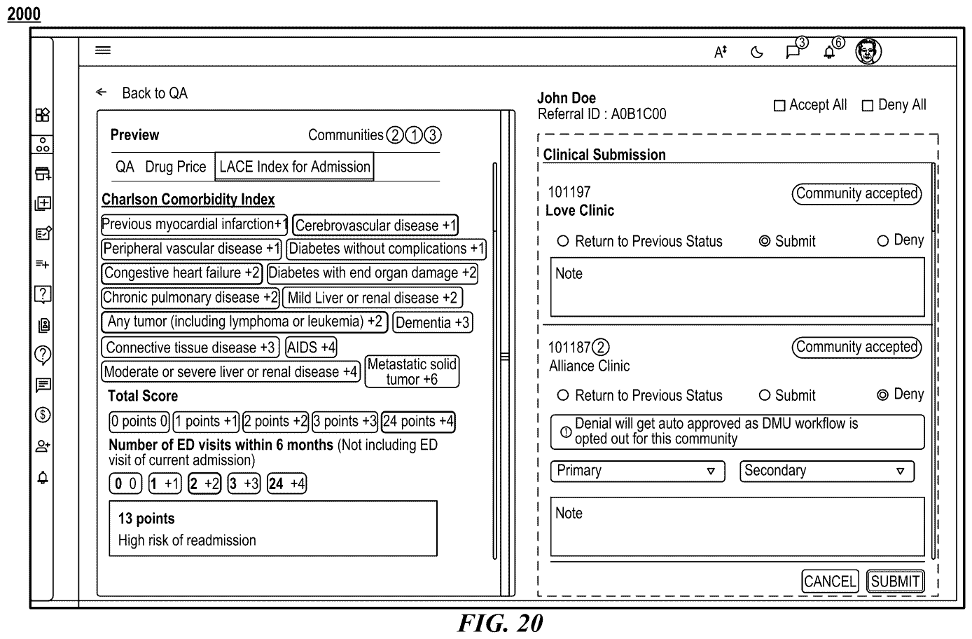

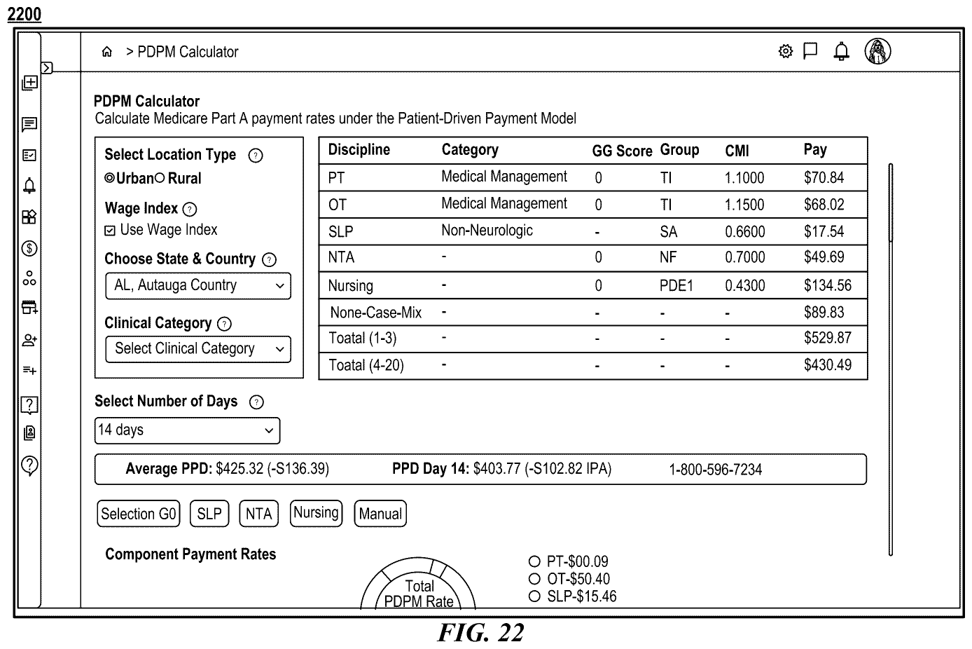

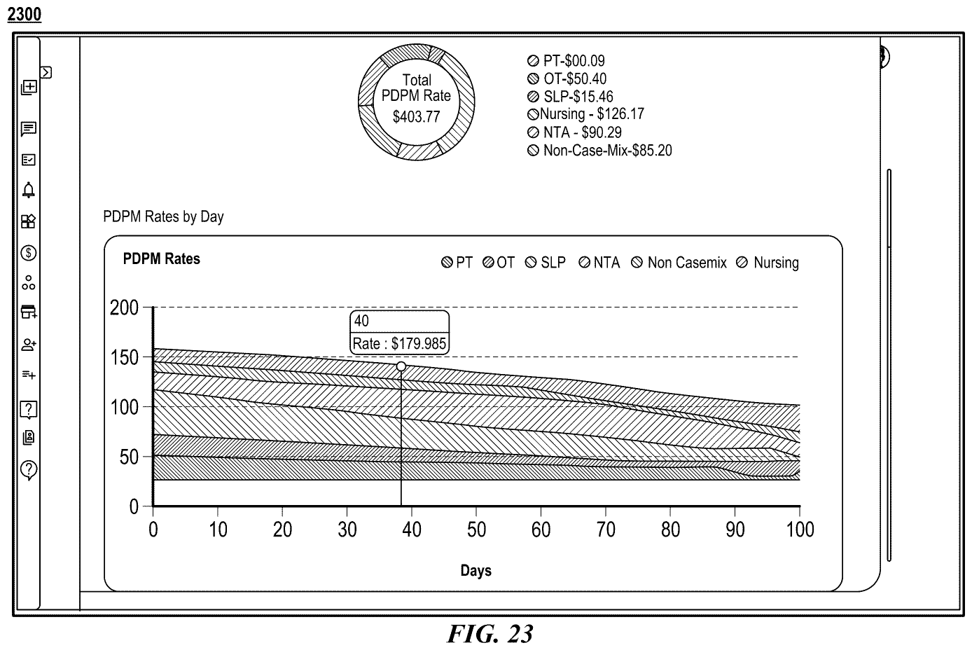

For each received data package, the system determines a priority score. These priority scores govern the processing order of the multiple data packages. The priority score calculation considers multiple factors. In healthcare settings, these factors include medical condition urgency indicators, proximity to service providing nodes, care facility occupancy levels, and alignment with care facility specializations. Specific modules for calculating these clinical and financial factors are detailed in subsequent figures. For example, an exemplary LACE Score System (Clinical Risk) is illustrated in FIG. 18, with exemplary user interfaces in FIGS. 19 and 20. An exemplary PDPM (Financial Forecasting) logic is shown in FIG. 21, with exemplary calculator interfaces in FIGS. 22 and 23. The system may assign higher priority component scores to medical care needs that are rarely available compared to those that are abundantly available. This weighted scoring ensures optimal allocation of limited specialized resources.

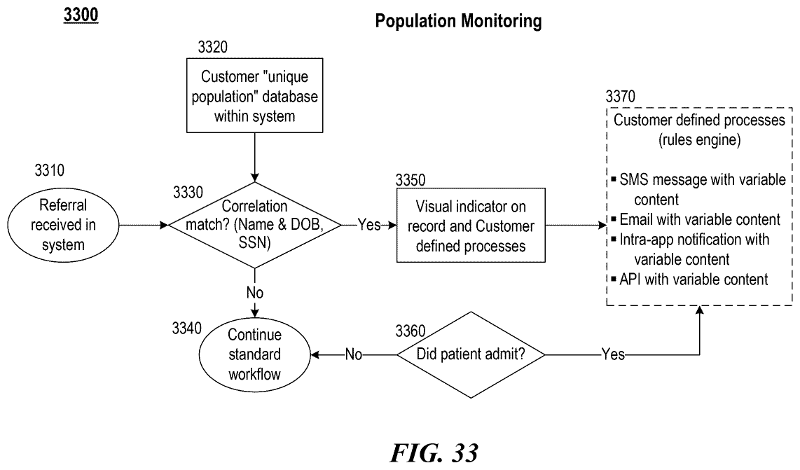

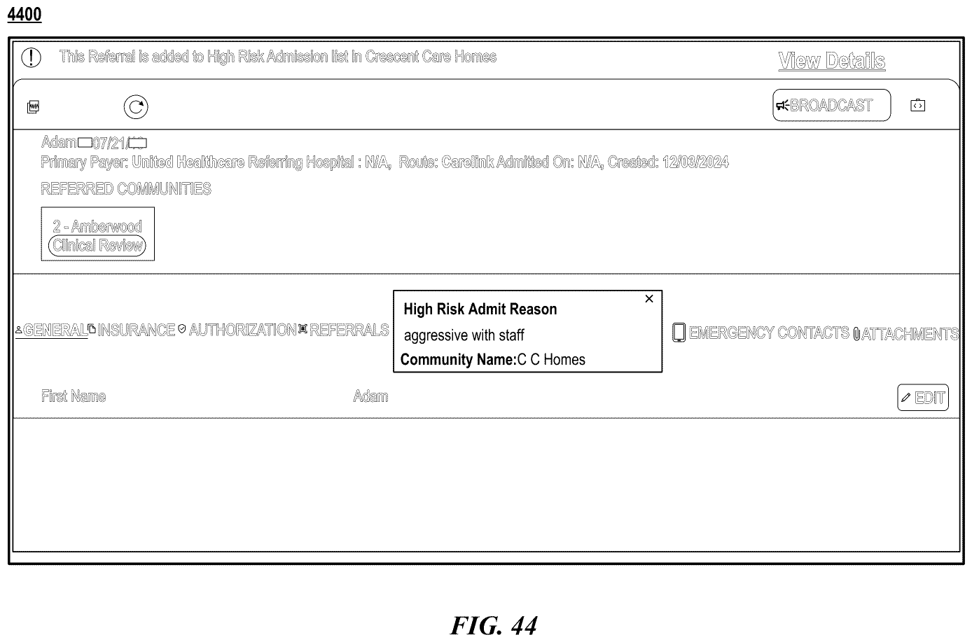

The system dynamically adjusts priority scores based on temporal factors. Each data package's priority score is adjusted based on the elapsed time since its receipt. This temporal adjustment helps prevent older data packages from being indefinitely superseded by newer ones with similar initial priority scores. The adjusted priority scores of data packages pending processing are synchronized across all processing nodes in the system, ensuring consistent prioritization across the distributed processing network and enabling efficient workload distribution based on real-time priority assessments. Additionally or alternatively, the system may employ population monitoring logic as shown in FIG. 33 to trigger alerts, such as the High Risk Admission Alert shown in FIG. 44.

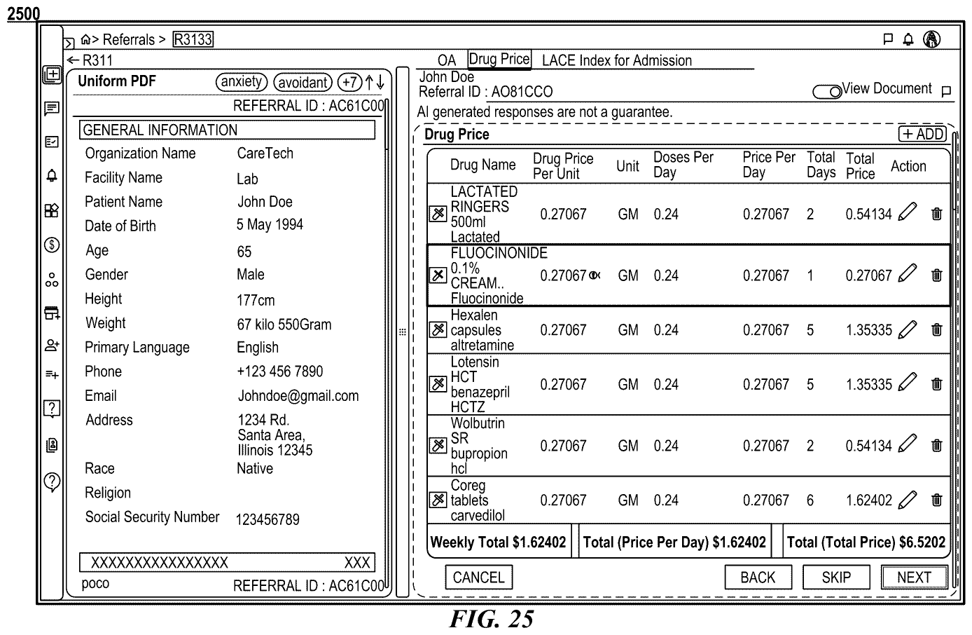

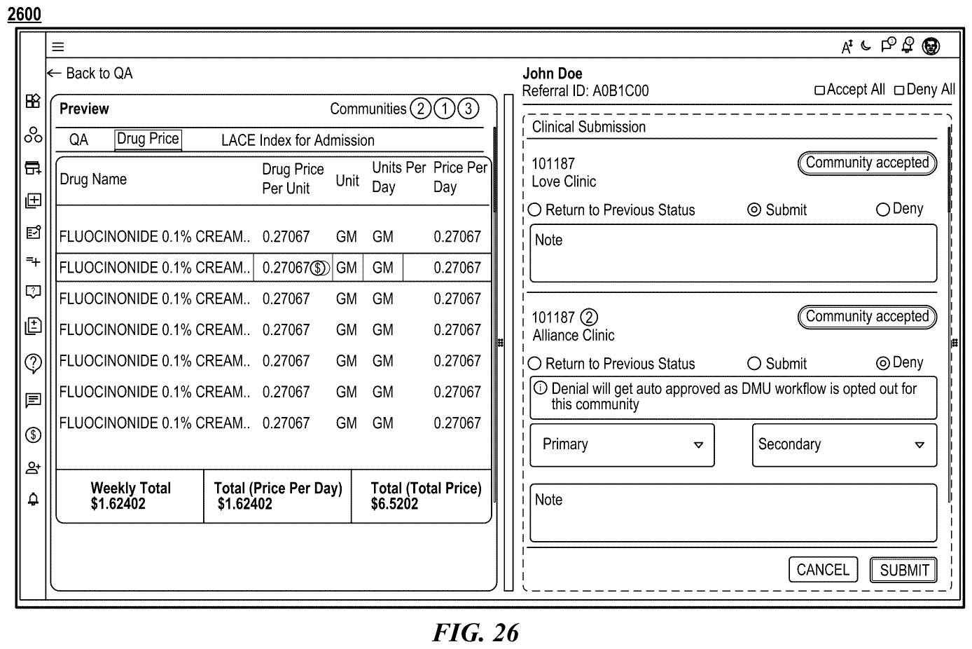

Upon receiving an acceptance from a target node, regardless of which entity—system or source node—initially receives the target node's response, the receiving entity automatically notifies the other to ensure data consistency across the distributed architecture. If multiple target nodes accept a same invitation, the source node or system determines the final assignment based on, e.g., their rankings. The ranking may consider factors such as response time, geographical proximity, capability matching scores, costs, or the like, or a combination thereof. Cost estimation may be supported by the Medication Cost Logic (FIG. 24) and Medical Equipment RAG System (FIG. 28), with exemplary interfaces shown in FIGS. 25 and 26.

For a chosen acceptance, the source node or system initiates approval processing. The system updates the centralized records to reflect the approved status. The source node or system notifies other target nodes that their invitations are no longer active. The system maintains a complete audit trail of all responses and status changes in the centralized database.

The system tracks both acceptances and denials in real-time. For denials, the system captures denial reasons and can trigger additional candidate node recommendation or identification if needed. All status updates are synchronized across the distributed processing network, ensuring all authorized users have access to current status information. Historical response patterns and processing outcomes are stored for analytics and future optimization of target node selection.

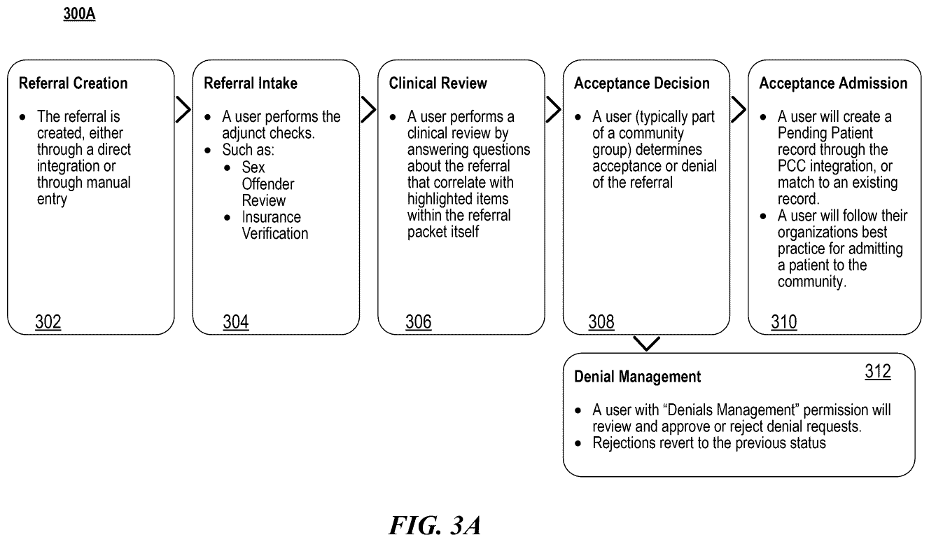

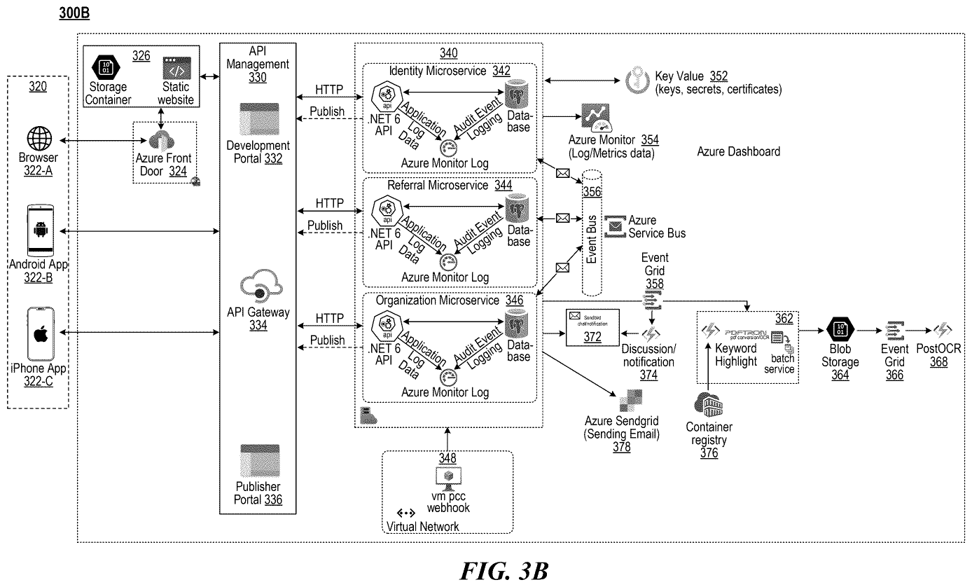

FIG. 3A illustrates a workflow of operations performed by a data processing system, according to some embodiments of the present technology. The workflow 300A shows an exemplary implementation of the process 200A/200B/200C shown in FIG. 2A/2B/2C in an exemplary use case of patient referral.

At stage 302 (Referral Creation), corresponding to operation 210 in FIG. 2, the system creates referrals either through direct integration with other systems (e.g., CarePort or NaviHealth), direct fax intake, or manual entry. This standardizes the referral structure regardless of the source.

Stages 304 (referral intake) and 306 (clinical review) correspond to operations 220 and 230 in FIG. 2. At stage 304 (Referral Intake), adjunct intake checks are performed. These include specific criteria review (e.g., sex offender status) and insurance verification through API integrations.

At stage 306 (Clinical Review), one or more of the following may be performed:
a. A clinical review is conducted by a reviewer to answer pre-determined questions related to the referral.
b. The questions are designed to assess the referral's needs and eligibility for services. The questions are customized based on community configuration and needs.
c. The system ingests attached documents using OCR to extract text (via the document processing engine 420 shown in FIG. 4), which is processed to identify keywords based on a dictionary of keywords (which can be customized). The extracted text is displayed to the reviewer with the highlighted keywords (via the verification rendering engine 460 shown in FIG. 4). This helps reviewers identify portions of the extracted text to answer specific clinical questions, or alternatively to validate AI/LLM automatically generated answers (discussed with reference to 220 and also below). In addition, specific highlight colors can be used for different groups of keywords in different categories, which can correspond to different categories of questions in the clinical review. The extraction and processing may utilize the PDF Processing Logic illustrated in FIG. 6. An LLM can be used to identify words synonymous with keywords, or even sections that are contextually relevant.
d. An LLM is used to process the extracted text to generate initial answers to the questions. This includes providing as an input to the LLM a prompt including instructions to act as a referral reviewer, a specific clinical question, extracted text data (or text chunk embeddings retrieved from the vector store index 434 shown in FIG. 4), community specific data (e.g., capabilities), and indication of input data format, and indication of requested output data format. See FIG. 10 for an exemplary RAG architecture and FIGS. 7 and 8 for an exemplary prompt engineering logic supporting this step.
e. Data related to the referral (e.g., needs) is also compared to community specific capabilities to determine whether a referral should be rejected and a notification displayed to the user. This is currently done by answering the clinical questions based on referral information from the referral packet. This corresponds to the capability matching logic shown in FIG. 14.

In some embodiments, a recommendation for another community in the organization can be determined and sent back to the referral source, if no referred community in the organization is able to accept the referral but a community exists in the organization that is a good or better fit. As an illustrative example, it can determine that a referral cannot be taken at (e.g., is rejected for) referred communities 1, 2, and 3 but that community 4 may be a good fit.
f. Referral can be compared with an org-wide high-risk admission list. If referral is on the list, a notification is displayed to the user along with relevant information related to rejecting the referral (e.g., reason for being on the list), and a way to short-circuit the process and reject the referral. FIG. 44 shows exemplary alerts.
g. Answers to clinical review questions are shared with other communities in the organization, ensuring that work is not duplicated, and only new questions for communities need to be answered/validated for additional clinical reviews conducted by other communities.

Stages 308 (acceptance decision), 310 (acceptance admission), and 312 (denial management) correspond to operation 240 in FIG. 2A. At stage 308 (Acceptance Decision), a user, e.g., part of a community group, reviews the referral and ultimately makes a decision to accept or deny the referral based on the clinical review and other relevant factors. This decision may be informed by clinical risk assessments from the LACE module (FIGS. 18-20) and/or financial forecasts from the PDPM module (FIGS. 21-23).

At stage 310 (Acceptance/Admission), one or more of the following may be performed:
a. if a referral is accepted, the system creates a “Pending Patient” record in the electronic medical record (EMR) or electronic health record (EHR) (e.g., Point Click Care (PCC)) integration or matches to an existing record.
b. this triggers automated responses to the referral source.
c. the appropriate organization follows their practices (e.g., best practices) for admitting the patient to the community through their EMR/EHR.

At stage 312 (Denial Management), if a referral is denied, one or more of the following may be performed:
a. a user with “Denial Management” permission reviews and approves/rejects a denial request.
b. a denial rejection reverts the referral to its previous status.
c. an approval triggers automated responses to the referral source.

FIG. 3B illustrates an architectural implementation of a data processing system 300B, according to some embodiments of the present technology. The system 300B may include the specific engines and processing flows illustrated in FIG. 4. The system 300B may be implemented using a serverless architecture (e.g., Azure Functions) where discrete processing steps are triggered by asynchronous events, as further detailed in the Data Ingestion Logic of FIG. 5. The system 300B receives data through input interfaces 322 (individually identified as web browser interface 322-A, mobile application interfaces 322-B, iOS application interface 322-C), phone, fax, email, etc. The input interfaces 322 enable (e.g., one- or bi-directional) data communications with multiple source nodes (e.g., hospital discharge planning systems).

The web browser interface 322-A communicates through a gateway service platform 324 that manages and routes web traffic by receiving incoming requests, managing connection security and protocols, and optimizing data transmission through caching. For example, such functionality may be implemented using cloud-based services like Azure Front Door. The gateway service platform 324 connects to a static content management component 326 to facilitate data transmission between the web browser interface 322-A and the API management component 330. The static content management component 326 may include a storage container configured to store website assets and a static website hosting element configured to serve static web content. The storage container of the static content management component 326 may be cloud-based storage. The gateway service platform 324 and the static content management component 326 may facilitate data communications via the web browser interface 322-A by providing server-hosted content (e.g., HTML/CSS/JavaScript content), content delivery optimizations (e.g., caching and routing), session management and load balancing, or the like, or a combination thereof.

The mobile application interface 322-B and iOS application interface 322-C communicate directly with the API management component 330. The mobile application interface 322-B and iOS application interface 322-C may be pre-compiled with built-in interface components for structured API communications with the API management component 330.

The API management component 330 may provide structured communication interfaces to manage integrations with external verification services. The API management component 330 may include a development portal 332 for API documentation and testing, an API gateway 334 for request routing and protocol handling, and a publisher portal 336 for API lifecycle management and monitoring. The API gateway 334 may perform verification operations through API integration as described in FIG. 2 and stage 320 in FIG. 3A. While the web-based and mobile/iOS communications follow separate paths (via 326 and 326 or directly connected to 330), all verification operations are centralized through the API management component 330.

In the example use case of patient referral, patient identity and status verification is performed through the API management component 330. When a data package is received, the API gateway 334 executes verification API calls to external services (e.g., for sex offender status checks). API management component 330 may attach the verification results to the data package. The verification results flow through the event bus 356 to the relevant microservices for processing decisions. All verification attempts and results may be logged and collected by the monitoring component 354.

The microservice cluster 340 includes an identity microservice 342, a referral microservice 344, and an organization microservice 346. The cluster 340 receives incoming requests through the API management component 330, which routes traffic from web browser interface 322-A (via gateway service platform 324 and static content management component 326) and mobile interfaces 322-B and 322-C. The API gateway 334 handles the routing of requests to appropriate microservices within the cluster 340, while the development portal 332 and publisher portal 336 provide API documentation and lifecycle management for interfacing with these microservices. The cluster 340 implements a layered security model where the identity microservice 342 validates authentication for requests before they are processed by the referral microservice 344 or organization microservice 346, with inter-service communications coordinated through an event bus 356 (e.g., Azure Service Bus).

The identity microservice 342 serves as a security and authentication component in the system architecture. It performs multiple types of verification including, e.g., input authentication for users submitting data packages, processing authentication for users reviewing and modifying analysis records, action authentication for users making decisions on referrals, and system access authentication controlling feature access based on user roles and permissions.

On the upstream side, the identity microservice 342 processes authentication requests and user identity data received through the API management component 330 from both web-based (via 322-A, 324, 326) and mobile interfaces (322-B, 322-C). It validates credentials of users accessing the system and ensures appropriate permissions for their requested operations.

The identity microservice 342 generates various categories of operational records during its processing: infrastructure monitoring data capturing system performance metrics, application logs recording service operations and API activities, and database transaction logs documenting data access patterns. These operational records are collected and processed by the monitoring component 354 for system analysis and auditing.

When the referral microservice 344 or organization microservice 346 receives a request, they interact with the identity microservice through the event bus 356 for authentication validation and access permission verification. The identity microservice 342 performs these security checks using credentials securely stored in the key value vault 352 and communicates decisions back through the event bus 356. The credentials may be in the form of keys, secrets, certificates, etc.

The security architecture may follow a token-based model. After initial authentication, security tokens may be used for subsequent service requests. Different user types (administrators, reviewers, facility staff) may have different access levels enforced through role-based access control. Each microservice may validate these tokens through the identity microservice, with all security-relevant actions recorded in operational logs. The monitoring component 354 may aggregate these records for security analysis and alert generation.

During system operations, such as document processing through a document processing component 362 or notification handling via components 374 and 378, the identity microservice 342 records all security-relevant events. The event bus 356 ensures timely distribution of security events throughout the system, enabling consistent security state management and rapid security incident response.

The referral microservice 344 implements the processing operations, e.g., operations 220 and 230 described in FIG. 2A/2B and operations at stages 304, 306, and 308 in FIG. 3A. The referral microservice 344 may have a similar internal architecture of the identity microservice 342, with its own infrastructure monitoring, application logging, and database transaction logging capabilities.

On the upstream side, it receives data packages routed through the API management component 330 and validated by the identity microservice 342. After receiving validated requests, the referral microservice 344 implements the analysis record generation described in operation 220 of FIG. 2A/2B or stage 306 in FIG. 3A. It interfaces with the document processing component 362 to perform the document processing operations detailed, including optical character recognition, keyword identification, and text highlighting of clinical review content. For example, the document processing component 362 may operate by executing PDFTron software. The referral microservice 344 may include a machine learning component as described in 220 of FIG. 2A/2B to generate the analysis record.

The referral microservice 344 performs the compatibility analysis described in operation 230 of FIG. 2A/2B and stage 306 of FIG. 3A. It evaluates capability matches and generates compatibility indicators through a series of processing steps, with results routed through the event bus 356.

For the acceptance decision process described in stage 308 of FIG. 3A, the referral microservice 344 coordinates with the discussion/notification component 374 and email service 378. It maintains processing state through the entire workflow outlined in FIG. 3A, from intake verification through final disposition. All operations are tracked through infrastructure monitoring data, application logs, and database transaction logs, collected by the monitoring component 354.

The referral microservice 344 works in conjunction with the organization microservice 346 to implement the multi-community processing capabilities described in FIG. 3A, with inter-service communications managed through the event bus 356. Security operations, including access control and audit logging, are coordinated with the identity microservice 342.

The organization microservice 346 manages organizational data and configurations needed by other services. The organization microservice 346 may have a similar internal architecture of the identity microservice 342 or the referral microservice 344, with its own infrastructure monitoring, application logging, and database transaction logging capabilities. Like the referral microservice 344, it receives requests through the API management component 330 after validation by the identity microservice 342.

The organization microservice 346 manages capability specifications for nodes (e.g., service providing nodes) in the network. These specifications include processing capacities, geographical data, resource availability, specialized handling capabilities, or the like, or a combination thereof. When the referral microservice 344 performs compatibility matching described in operation 230 of FIG. 2A/2B, it queries the organization microservice 346 through the event bus 356 to obtain current capability data (as stored in the node capability specifications of (potentially available) nodes (e.g., participating nodes within a node network) in the storage device 442 shown in FIG. 4). This enables real-time matching based on updated organizational capabilities.

In multi-node operations, the organization microservice 346 implements group management functions. It maintains node relationships, hierarchies, and processing rules. When the referral microservice 344 identifies candidate nodes during the matching process described in stage 306 of FIG. 3A, the organization microservice 346 provides organizational context such as geographical groupings and preferred routing patterns.

The organization microservice 346 manages organizational data through a structured database. For example, the organization microservice 346 manages organizational data including node configurations and relationships, resource allocation rules and thresholds, geographical service boundaries, processing priorities and specializations, organizational hierarchies and reporting structures, or the like, or a combination thereof.

The communication patterns between the API management component 330 and the microservice cluster 340 are represented by two distinct types of connections. Solid bidirectional arrows marked with “HTTP” indicate synchronous request-response communications where components interact directly through HTTP protocols. These HTTP connections are used between the API gateway 334 and the microservices (342, 344, 346), enabling immediate two-way data exchange.

Dashed unidirectional arrows marked with “Publish” represent publish-subscribe communication patterns. These may be asynchronous. These connections show how components publish events or messages to be consumed by other components. For example, the microservices publish events to the event bus 356, and the development portal 332 and publisher portal 336 publish API-related updates to the API gateway 334.

The combination of these communication patterns enables both real-time interactions and event-driven processing, allowing the system to handle both immediate requests and asynchronous operations efficiently through appropriate protocols.

For communication and notification functions, the organization microservice 346 interfaces with the discussion/notification component 374 and email service 378 through the event bus 356. It triggers organizational notifications, distributes configuration updates, and manages communication preferences at the organizational level.

The operations may be tracked through logging, with records collected by the monitoring component 354. The event bus 356 ensures that organizational state changes are consistently propagated across the service cluster, maintaining system-wide data consistency. The data in the monitoring component 354 may be organized for visualization and interaction via an observability portal (e.g., Azure Dashboard).

The system architecture includes several specialized components for data processing and event handling. A virtual network interface 348 enables webhook integration with external record systems (e.g., PointClickCare EHR). An event grid 358 implements rule-based event routing and filtering, while a separate event grid 366 manages rules for document processing events.

The communication infrastructure includes a notification API 372 (e.g., ChatNotification API) for messaging services and a container registry 376 for managing containerized applications. File management is handled through blob storage 364 (e.g., Azure Blob Storage), providing scalable storage for unstructured data. A post-processing function 368 performs operations on documents after optical character recognition processing.

These components work together through the event bus 356 to provide integrated functionality. The webhook-based integration enables real-time data exchange with external systems, while the event grids manage rule-based processing and routing of system events. The containerized application management supports scalable deployment, working in conjunction with the blob storage for handling unstructured data. The post-processing workflow automates document handling after initial recognition processes.

For example, the referral microservice 344 generates recommendation messages which may be routed through the event bus to the notification API 372. The notification API 372 processes the recommendations through the discussion/notification component 374. Based on source node configuration (web-based via 322-A or mobile via 322-B, 322-C), the communication is routed either through the web gateway 324 or directly through the API management system 330. For web-based interfaces, the static content management system 326 provides presentation resources.

The system supports real-time updates through its event-driven architecture. Event grid 358 manages notification rules and triggers, while event grid 366 handles document-related events. Status updates and responses are synchronized through these event grids, ensuring consistent information display across all interfaces.

The communications are validated through the identity microservice 342, with operation logs maintained in blob storage 364 and monitored through component 354. This ensures secure, traceable communication while maintaining data consistency between the source node and the system.

The microservice architecture provides several technical effects and/or advantages through its structural arrangements. The division into distinct services (identity microservice 342, referral microservice 344, and organization microservice 346) within a unified cluster 340 enables independent scaling and deployment of each service while maintaining coordinated operations through the event bus 356.

The centralized identity microservice 342 provides consistent security management across the system. By validating all authentication requests and maintaining comprehensive operational records, it ensures uniform security enforcement while simplifying security auditing and compliance tracking.

The separation between referral microservice 344 and organization microservice 346 enables specialized handling of different data types while maintaining system cohesion. The referral microservice 344 can focus on document processing and workflow management, while the organization microservice 346 handles capability specifications and resource management.

The shared monitoring infrastructure, where each microservice generates operational records collected by component 354, enables comprehensive system visibility while maintaining service independence. This arrangement facilitates problem detection and performance optimization across the distributed system.

The integration through the event bus 356 enables loose coupling between services while ensuring reliable message delivery. This architecture supports system evolution, as individual services can be updated or modified without impacting other components, provided they maintain consistent event interfaces.

The following description details a workflow implementation in an example use case of patient referral. The workflow expands upon the operations shown in FIG. 2 (210-240) and stages illustrated in FIG. 3A (302-312), with specific technical implementations shown in the architectural components of FIG. 3B. The workflow encompasses several integrated functionalities including the following.

1. Referral Capture and Aggregation. The operation corresponds to 210 of FIG. 2 or 302 of FIG. 3A. Referral packages may be received through input interfaces 322 and API management component 330.

The system (e.g., 150, 300B) may integrate with various referral channels, such as a NaviHealth, Careport, etc., (e.g., discharge planning and referral software systems used by acute care providers) as well as direct fax intake, email intake, and an outward facing patient portal for use by physician offices. The system aggregates all the referrals from multiple sources and standardizes the structure for consistency. Referrals are aggregated for multiple communities within an organization to minimize duplicate work.

2. Automated Intake Reviews. The operation corresponds to 210 or 220 of FIG. 2 or 304 of FIG. 3A. The automated intake review may be performed via API gateway 334 verifications. The system utilizes APIs for sex offender review and insurance verification, with the goal of incorporating criminal background check and asset search capabilities.

3. Clinical Review Keyword Highlighting and AI Processing. The operation corresponds to 220 of FIG. 2 or 306 of FIG. 3A. The operation may involve document processing component 362 and the referral microservice 344.

The system involves processing referral packet documents with Optical Character Recognition (OCR). Keywords are identified and highlighted (e.g., by the verification rendering engine 460 of FIG. 4) based on a curated dictionary of keywords using fuzzy matching logic, allowing users to streamline their review and answer clinical questions effectively. Different colors indicate various categories of questions, facilitating easier navigation and categorization. Additionally, a Large Language Model (LLM)/Natural Language Processing (NLP) process may be employed to identify words or sections that are contextually relevant to the keywords. For instance, a prompt containing text from the referral packet can be input into the LLM with a task to identify and return a list of words or segmented text chunks that either match, are synonymous with, or are semantically relevant to a provided list of keywords for a particular category. These returned words and text chunks are then highlighted in the referral packet for user review, using different colors for each category. This approach helps highlight relevant text related to the category that may not be direct keyword matches, instead focusing on logically and contextually relevant sections based on all keywords from the category. The extraction and processing may utilize the PDF processing logic illustrated in FIG. 6 and the paragraph tagging logic of FIG. 7. In simpler applications, this process can identify and highlight various keyword synonyms in addition to the original keywords for the category.

Document data is provided to the LLM with organization or community-tailored prompts to extract context for filling out clinical questions. The LLM leverages data within the provided documentation to assist with the clinical review process, utilizing a default set of questions and prompt pairs. Additional clinical questions provided by the community have custom LLM prompts engineered by the internal team to ensure the most appropriate responses. Each question has a corresponding custom prompt used in combination with the referral packet, essentially forming a table of questions and corresponding LLM prompts. For example, the community may have a question to ascertain a patient's past medical history, and the generated AI prompt used by the LLM may be: “Summarize the details of the patient. Here are the specific aspects to include in your summary: Primary referral reason/diagnosis; Recent medical issues; Current medications; Past medical history (PMHx) with a focus on items that may impact current care needs; Any recent tests or procedures; Important symptoms or findings. Ensure that your summary focuses on the most critical information necessary for the patient's ongoing care, omitting any identifying details such as the patient's name or birth date and do not provide treatment recommendations or considerations.”

The LLM provides responses that answer the questions posed in the prompts, following guidelines such as selecting between one or more options or providing freeform answers. It utilizes the referral packet and other related information provided to the LLM concerning the referral. The prompts may include instructions to either leave the response blank or indicate in the question response text input field if the referral packet lacks sufficient information to answer the question. They may also set logic flags indicating that the question was unable to be answered, which can trigger the display of specific icons or text adjacent to the question, indicating that the LLM was unable to answer. Additionally, prompts may indicate if the LLM may only partially answer the question and whether additional information is needed.

A method of source attribution, such as Retrieval-Augmented Generation (RAG), can also be used to indicate what specific information in the referral packet was used to answer each question. This involves performing a pairwise correlation determination between a query embedding (derived from the question) and text chunk embeddings (derived from the referral) to identify relevant text chunks, as managed by the vector embedding module 432 of FIG. 4. This can be done by providing a link to a specific section of the referral packet or, alternatively, in a tooltip or adjacent to the question, directly quoting text from the referral packet. The link may direct to the portion of the text generated from OCR from the referral packet (which may be a PDF or image) or to a specific location in the referral packet PDF file. The identified text is mapped to a location in the PDF. FIG. 12 illustrates an exemplary visual correlation system block diagram.

Various icons are used (e.g., placed adjacent to the questions) to denote the status of the answers. For example, one icon may indicate that the answer is LLM-generated, another icon may signify that the answer was LLM-generated but human-edited or reviewed, and a third icon—or the absence of an icon—may indicate that the LLM was unable to provide an answer.

These integrated features collectively provide a robust solution for managing referrals, enhancing clinical reviews, and ensuring consistency and efficiency across multiple channels and communities within an organization.

4. Multi-Site Referrals. The operation corresponds to 230 and 240 of FIG. 2A or stage 306 of FIG. 3A. The multi-site referral may the referral microservice 344 and the organization microservice 346. If a referral is referred to multiple communities there is still only a single referral with multiple referred communities. This creates a centralized referral and minimizes duplicate work. Within an organization once one community fills out the clinical review questions for a particular referral, the same question is automatically filled out for other communities. Only community-specific questions need to be completed for each additional community.

5. Capacity Matching. The operation corresponds to 230 of FIG. 2A/2B or stage 306 of FIG. 3A. The capacity matching may involve the referral microservice 344 and the organization microservice 346 (and the candidate matching engine 440 of FIG. 4).

Capacity matching can be executed on a question-by-question basis. Organizations and communities establish a capability rating for each answer option for every question, e.g., using colors such as “red,” “yellow,” or “green.” For example, a red rating signifies a referral-need community-capability mismatch (e.g., failure to satisfy a mandatory constraint), acting as a hard stop for the community; yellow indicates that further review, consideration, or verification may be necessary (e.g., failure to satisfy a preferred constraint), while green denotes that there are no issues.

Referral patient needs are compared with community site capabilities, which may include resources like ventilators, bariatric care, complex wound treatments, or facilities for patients needing a locked dementia unit. Certain mismatches in these needs can lead to automatic rejection of the referral or alert the user to expedite the review process. Alerts related to red and yellow capability ratings are displayed during the clinical review after the Large Language Model (LLM) processes each question. A top banner summary provides a count of red and yellow answers identified from the clinical review. This banner may also indicate the number of referred communities marked red out of the total number of referred communities within the organization and may suggest if there are non-referred communities in the organization that would be a good fit. The system considers communities based on organizational configurations, such as similar geographical locations or ZIP codes, to determine which communities should be evaluated together.

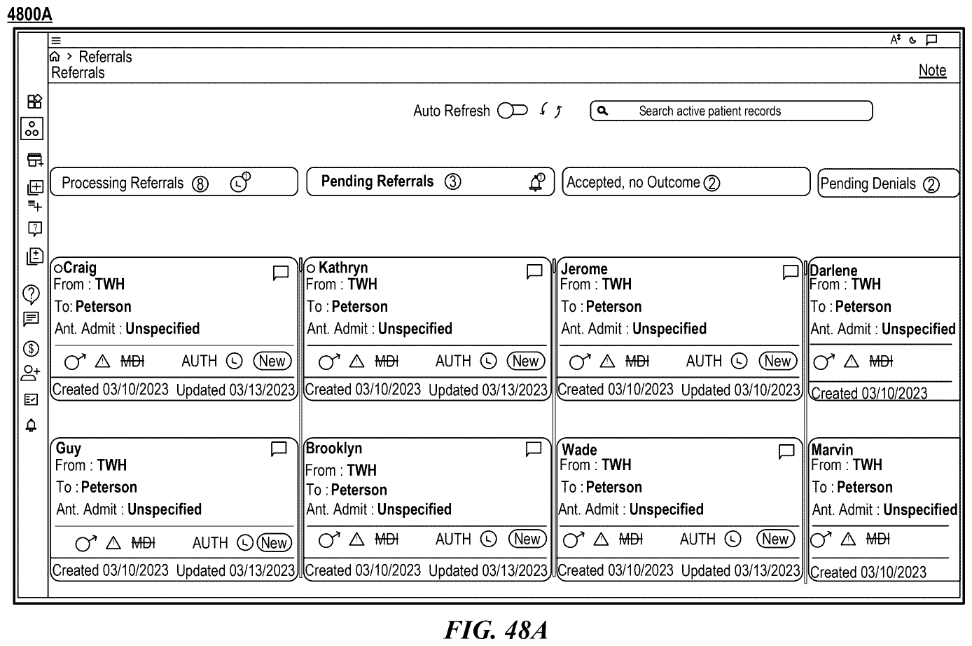

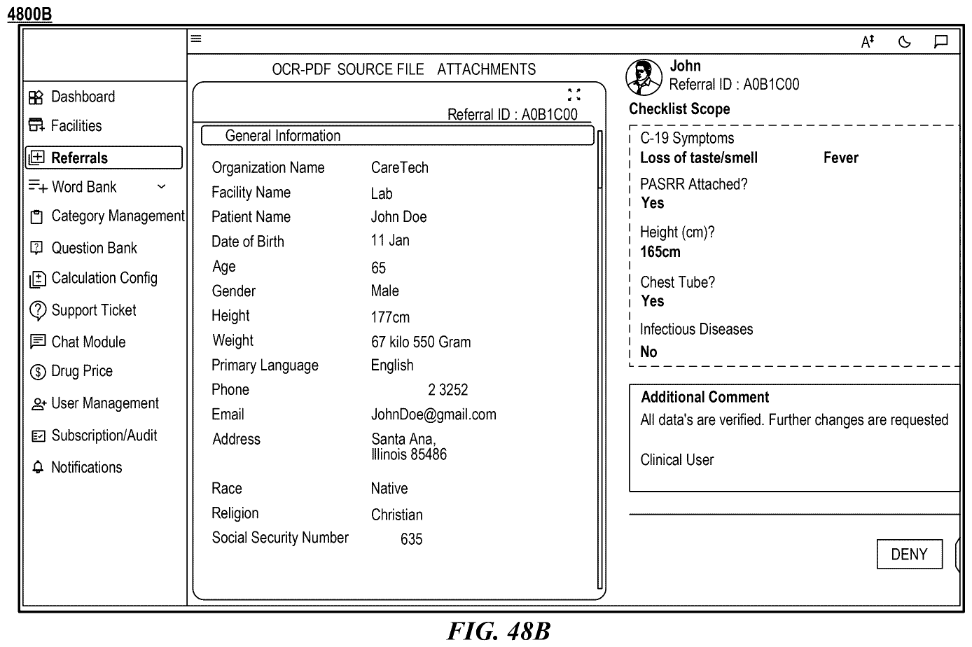

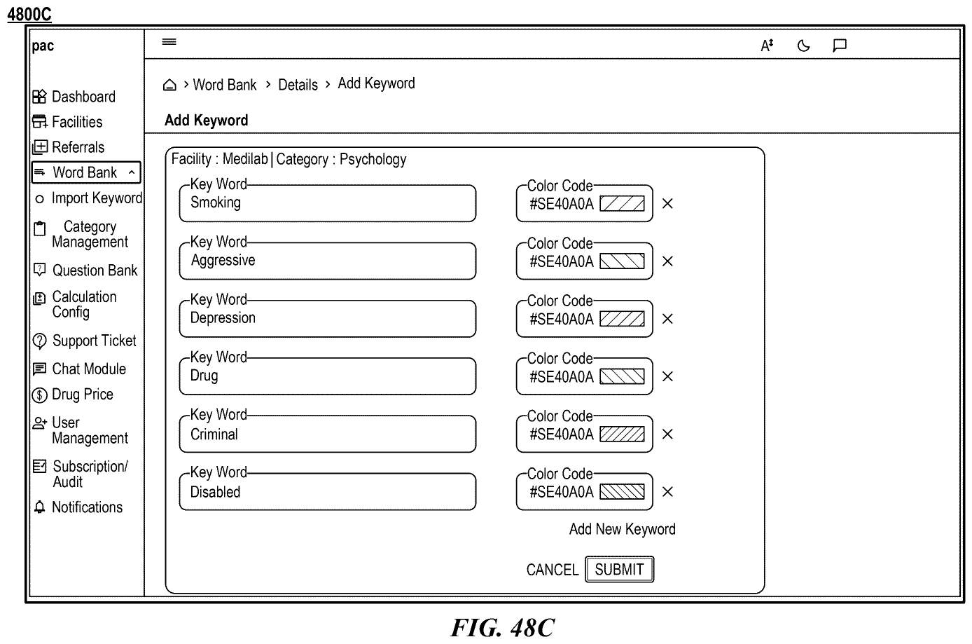

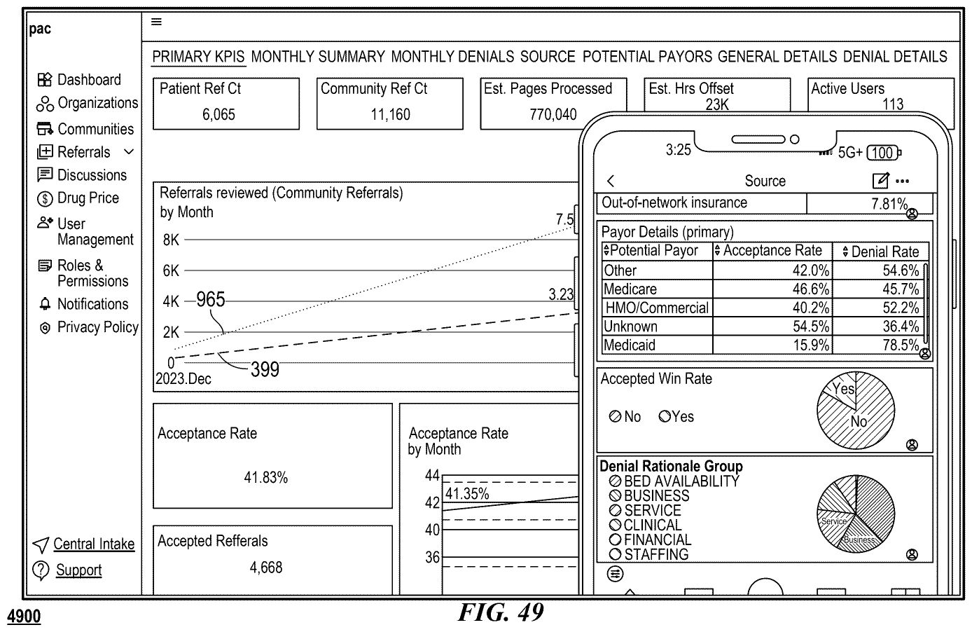

Similar alerts are shown in the post-clinical (community) review phase. These alerts are specific to each community's capabilities and can include additional review questions and answers. Each community may have a different capability rating (e.g., color) associated with specific question answers, indicating whether the community can handle the needed capability. See FIGS. 48A-48C (Clinical Review UI) and FIG. 49 (Community Review UI) for example interfaces.

Patient needs for care are either pulled by the AI system or determined through clinical review and are then compared to a community-provided capability grid that outlines each community's ability to care for the patient. This comparison is currently conducted on a question-by-question basis during the clinical review. For example, if a patient needs a locked dementia unit for safe care and the AI or user flags this need, a “mismatch” flag is displayed to notify the user of the discrepancy. This capability matching also applies when a patient is a registered sex offender and the community is unable to admit them, if the community lacks a contract with the patient's insurance, or if there are no available beds for placement based on occupancy, gender, or isolation status.

Capability matching may also be performed for non-referred communities to determine if there are any additional communities within the organization that would be a suitable fit, meaning no capability mismatches are identified. This matching can be done alongside referred communities or after determining that existing referred communities are bad fits due to “hard-stop” capability mismatches. When sending a denial response back to the referral source, the system may include a recommendation for a better-fit community based on matched capabilities in a “notes” text input field. For example, the note might state, “Unable to accept at [communities 1, 2, and 3], but please open referral for [community 4].” These notes may include reasoning, such as the lack of capability at denied communities but the presence of necessary capabilities at the recommended community. Once the referral source submits a new referral for the recommended community, the system maintains the existing information. Consequently, any completed intake reviews and previously answered clinical questions may be prepopulated, ensuring a seamless and efficient referral process.

6. Prioritized Referral Scoring. The operation corresponds to 230 of FIG. 2A/2B or stage 306 of FIG. 3A. The capacity matching may involve the microservice cluster 340.

Prioritized Referral Scoring involves assigning a prioritization score to each referral to determine the order in which referrals are addressed. Each referral is initially given a score based on the primary payer, which varies depending on the organization or community. This score is subsequently adjusted according to the time elapsed since the referral was submitted by the referral source; the score increases the longer the referral remains unaddressed to enhance its prioritization.

In addition to the primary payer and time factors, several other potential factors influence the prioritization score. These include the patient's clinical conditions, the proximity of the referral to the community's location (with closer referrals receiving higher scores and those further away receiving lower scores), the patient's age, the primary diagnosis, and whether the patient is considered high-risk. For instance, a high-risk patient may have their score deprioritized because they are more likely to be rejected.

Furthermore, expedited requests, as indicated by the referral source, may have their scores increased to ensure they are moved to the top of the priority list. This comprehensive scoring system ensures that referrals are handled efficiently and that the most critical and time-sensitive cases receive appropriate attention promptly.

7. Shared High-Risk Patient Admission List. The shared high-risk patient admission list may be maintained through the organization microservice 346 and distributed via the event bus 356. The list may be used in 210 or 220 of FIG. 2A/2B, or 304 of FIG. 3A.

Shared high-risk patient admission list is an organization-wide feature that allows users in different communities within an organization to add patients along with specific concerns, such as a history of problematic behavior. This high-risk patient list can be utilized to compare against incoming referrals across various communities within the organization. When a referral matches an individual on the high-risk patient list, the system notifies the reviewer by displaying an alert on their referral page or by showing an alert indicator icon on their referral card in grid view. Additionally, if a referral is identified as a high-risk patient, the system may deprioritize the referral by adjusting its prioritization score lower. This ensures that high-risk patients are managed appropriately and that reviewers are promptly informed of any potential concerns related to the referrals they are processing.

8. Distributed/Virtual Central Intake. The operation corresponds to 210 of FIG. 2A/2B or 302 of FIG. 3A. The distributed/virtual central intake may be enabled by the microservice architecture 340 and event bus 356 communication.

The system enables staff at multiple communities within the organization to share the processing of intake referrals. This allows community specific users to share the referral intake workload across communities to ensure referrals are processed quickly and efficiently. The system allows authorized and credentialed users, including central intake, community-specific, and regional leadership users, operate within the same system.

9. Data Driven Actions. The operation corresponds to 240 of FIG. 2A, or 310 or 312 of FIG. 3A. The actions may be supported by monitoring component 354 and analytics capabilities across the system 300B.

Comprehensive dashboards and user notifications are integral features of the solution, providing detailed insights and actionable information for both communities and the organization as a whole. The comprehensive dashboards display granular data covering various aspects such as staffing, volume, sources, user actions, outcomes, denials, and admissions. This extensive data visualization allows organizations to monitor and analyze their operations effectively, ensuring that all critical metrics are tracked and managed efficiently.

In addition to the dashboards, the system is designed to notify users with insights based on the collected data. For instance, users receive alerts when processing times for a particular clinical question exceed a defined threshold, ensuring that delays are promptly addressed. Similarly, notifications are sent if the number of denials surpasses a specified limit or if the volume of referrals exceeds set thresholds. These timely notifications enable users to respond quickly to potential issues, maintain optimal performance, and uphold high standards of service across all communities within the organization.

This solution addresses the inefficiencies and complexities of the current referral intake process, reducing labor costs through deduplication of work, minimizing errors, increasing decision speed, and improving overall quality of care. It also creates a whole new system.

FIG. 4 illustrates a system block diagram of a centralized processing hub or system 400, according to some embodiments of the present technology. The centralized processing hub 400 (“system 400”) may correspond to the processor configurations within system 150 or 300B. The system 400 may include a set of distinct functional engines configured to execute the specific intake, analysis, and verification operations described herein. These engines may be implemented as software modules executed by at least one processor, as serverless functions (e.g., Azure Functions), or as dedicated hardware components.

As shown in FIG. 4, the system 400 includes one or more components including a data ingestion engine 410, a document processing engine 420, a machine learning (ML) engine 430, a candidate matching engine 440, a decision support engine 450, a verification rendering engine 460, an additional documentation request (ADR) management engine 470, and a rules engine 480.

The data ingestion engine 410 may be configured to serve as the entry point for data packages received from source nodes 110. The data ingestion engine 410 operates via the network interface to accept data packets including, e.g., unstructured files (PDFs, fax images), and structured data streams. In some embodiments, the data ingestion engine 410 utilizes an asynchronous architecture (e.g., an event grid or message queue) to decouple the receipt of data from immediate processing, ensuring high availability and scalability during peak referral volumes.

The data flows from the ingestion stage to a document processing engine 420. This engine is responsible for the “Digitization” phase. It executes an Optical Character Recognition (OCR) process on the document images to generate extracted text. The document processing engine 420 may also generate a coordinate map (e.g., x/y bounding box coordinates) that defines the precise spatial location of each extracted word or paragraph within the original image. This spatial data may be preserved and passed downstream to enable the visual verification features described later.

The ML engine 430 may be configured to synthesize the extracted text and generate a structured analysis record defining patient needs. The ML engine 430 may include one or more modules including a vector embedding module 432, a vector store index 434, a retrieval module 436, and/or an analysis generation module 438. The vector embedding module 432 converts portions of the extracted text (e.g., paragraphs or “text chunks”) into high-dimensional vector embeddings. These embeddings are indexed in a vector store index 434 (which may be a temporary, session-specific index). The retrieval module 436 (or RAG module) performs a pairwise correlation determination (e.g., cosine similarity search) between query embeddings (derived from clinical questions) and the text chunk embeddings to identify the most relevant text chunks. The analysis generation module 438 (utilizing, e.g., a large language model) synthesizes these relevant chunks to generate an analysis record containing specific compatibility indicators (e.g., “Patient requires Dialysis: Yes”).

When the patient needs are digitized into the analysis record, the data is processed by a candidate matching engine 440. This engine compares the generated compatibility indicators against node capability specifications stored in a database 442. The candidate matching engine 440 applies hierarchical constraint logic, distinguishing between mandatory constraints (where a failure triggers a “hard stop” or automatic exclusion) and preferred constraints (which influence ranking). This engine determines which service providing nodes are valid “Candidate Nodes.”

The decision support engine 450 may be configured to determine quantitative metrics used to assess, rank, and/or prioritize the candidate nodes identified by the candidate matching engine 440, to support comprehensive decision-making beyond simple capability matching. The decision support engine 450 integrates one or more specialized modules:

A clinical risk module 452 executes the LACE scoring algorithms shown in FIG. 15 to predict patient readmission risks.

A financial forecasting module 454 executes the PDPM logic illustrated in FIG. 18 to estimate reimbursement potential based on factors such as urban/rural designations and wage indices.

A cost estimation module 456 calculates specific medication and equipment costs via the pricing integration logic shown in FIG. 24. The system 400 utilizes the aggregated scores from the decision support engine 450 to identify one or more recommendations, and/or generate a ranked recommendation list, allowing the user to select the optimal facility based on a holistic view of clinical risk, financial viability, and/or operational cost.

The verification rendering engine 460 may be configured to generate an interactive user interface that visually correlates the analysis record with the original source documents. The verification rendering engine may combine the compatibility indicators from the ML engine 430 with the coordinate map from the document processing engine 420. It generates the verification interface (as illustrated in FIG. 2B, 250), which presents the user with a visual overlay. This overlay visually associates the AI-generated answers (e.g., “Diagnosis: Pneumonia”) directly to the highlighting of the relevant text chunks on the original document image, allowing for rapid human validation and auditing of the automated results.

The ADR management engine 470 may be configured to manage supplemental information requests generated by insurance payors during the billing cycle. The ADR management engine 470 integrates tightly with the data ingestion engine 410 and the document processing engine 420. While the upstream engines focus on referral intake and facility matching, this engine 470 addresses the “revenue cycle” phase. For example, insurance/payor organizations commonly deny claims and send a request back to the provider asking for more documentation to support the claim. Although this documentation is most frequently found within the original referral packet used for the first billable claim, providers often struggle to locate and assemble it. By leveraging the centralized data repository created during the intake phase, the ADR management engine 470 introduces a systematic and auditable process for satisfying insurer documentation requests, reducing administrative workload and ensuring regulatory compliance.

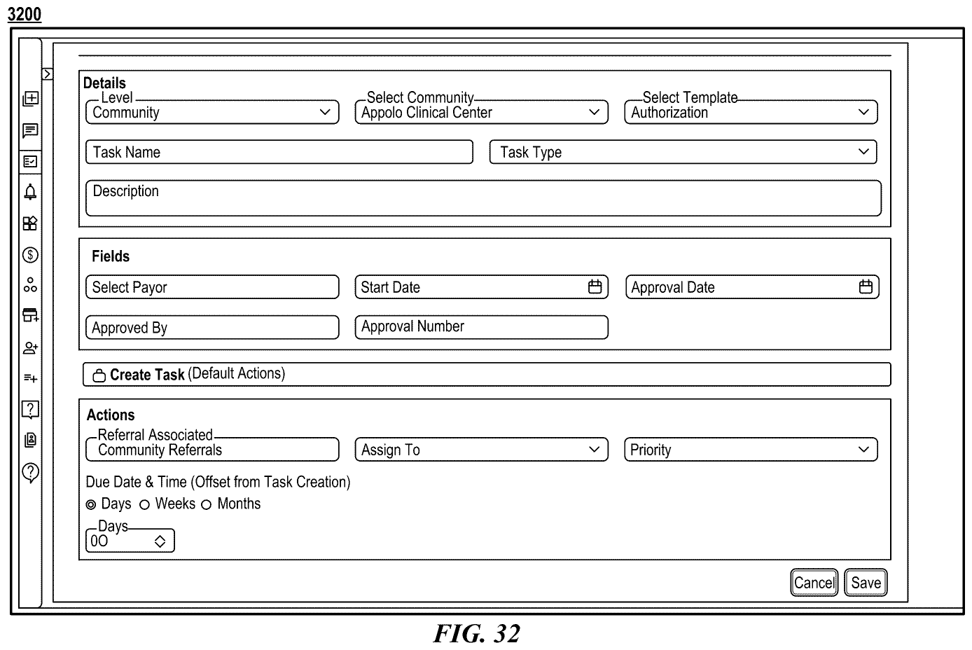

The information flow between these components is managed by the rules engine 480. The rules engine 480 operates on an “Event-Condition-Action” framework (as illustrated in FIG. 29) to orchestrate the processing pipeline. For example, upon detecting an “Ingestion Complete” event from engine 410, the rules engine 480 triggers the ML engine 430. As another example, upon detecting a specific capability mismatch from engine 440, the rules engine 480 may interrupt the flow to trigger a “High Risk Alert” (see FIG. 44) or generate an automated task (see FIG. 32). The rules engine 480 ensures that data transitions seamlessly between the intake, analysis, and decision-making phases based on configurable organizational protocols.

The functional engines illustrated in FIG. 4 may be implemented via the specific architectural components of FIG. 3B. The data ingestion engine 410 may correspond to the API management component 330 and gateway service platform 324, which manage the intake of data from the Input Interfaces 322. The document processing engine 420 may correspond to the document processing component 362 and post-processing function 368. The ML engine 430 may primarily be hosted within the referral microservice 344, which manages calls to external AI services (e.g., Azure OpenAI) and utilizes vector store indexes. The candidate matching engine 440 may be implemented by the referral microservice 344 in communication with the organization microservice 346 (hosting the “hard stop” data). The decision support engine 450 may correspond to specialized sub-routines within the referral microservice 344 or dedicated microservices (e.g., a pricing service) that execute the algorithms of the LACE, PDPM, and cost modules. The verification rendering engine 460 may correspond to the static content management component 326, serving the visual overlay interface to the user's web browser 322-A. The rules engine 480 may correspond to the event grid 358 and event bus 356 routing triggers to the referral microservice 344, which acts as the controller.

FIG. 5 illustrates a flow diagram for a community capability mapping, according to some embodiments. The process 500 provides an exemplary end-to-end view of how patient referrals are processed by the centralized processing hub 400 from intake to decision support.

The process 500 begins with patient referrals 510 received from various sources (e.g., Care Port, Navihealth, Manual Entry, Fax). These sources provide data packages 515, such as XMLs, HL7 messages, and attachments. In some embodiments, the process 500 includes executing a standardization step 516. Because the incoming data packages 515 may arrive in heterogeneous formats (e.g., raw fax images, XML data streams, or HL7 text), the process 500 may include processing these inputs to generate a uniform referral PDF. This step normalizes the disparate file types into a single, consistent document format.

The process 500 includes performing OCR 520 on the input documents to extract raw text. In some embodiments, the extraction includes a paragraph tagging and coordinate mapping process to facilitate downstream intra-document navigation. As the OCR engine processes the document, it assigns a unique identifier to discrete text blocks (e.g., paragraphs or lines or other discrete chunk of text) and captures their spatial coordinates (e.g., X/Y bounding box values) relative to the document page. This paragraph-level metadata is preserved alongside the extracted text. This tagging enables the interactive context linking and intra-document navigation (e.g., as described in Section III.c), where subsequent AI-generated answers can be hyperlinked back to these specific coordinates. For example, if the AI later cites a specific paragraph as evidence for a capability match, the system uses these stored tags to auto-scroll the user to that exact location and render a visual overlay (e.g., a highlight box) around the source text.

To manage load, the process 500 includes creating a queue message at 525, which activates an Azure function queue trigger at 530. This triggers the function to read, at 535, the processed document text (generated by OCR at 520).

The process 500 includes generating a payload at 540 by combining the document text and specific prompts for community capability mapping (CCM).

The process 500 includes accessing a data repository or storage 544 configured to support retrieval-augmented generation (RAG) operations. While illustrated in the context of RAG, the storage 544 is not limited to vector-specific stores; it may comprise a traditional database (e.g., a relational Structured Query Language (SQL) database), a non-relational (NoSQL) database, a knowledge graph, or a hybrid storage system. This storage 544 is populated with community capability mapping data for various organizations (e.g., Org 1 541, Org 2 542), which acts as the ground truth or knowledge base retrieved to augment the generative AI model's prompt payload.

The combined payload is sent to an AI Model at 545 (e.g., Azure OpenAI GPT 4.1 mini). In some embodiments, the combined payload includes both the referral data payload generated at 540 and the community capability mapping data retrieved at 544. By including the capability data directly in the prompt payload, the generative AI model is configured to simultaneously extract the clinical facts and perform the capability assessment, returning a structured output that includes the capability determination (e.g., “Pass/Fail”).

The system is not limited to this single-pass architecture. In some embodiments, the capability assessment logic may be decoupled from the extraction phase. For example, the payload sent to the AI model at 545 may include only the referral data and prompts for extracting clinical answers (e.g., “Does the patient require a ventilator?”). The system then receives the extracted answers and performs the capability assessment subsequently using a deterministic rules engine (e.g., traditional boolean logic or code-based comparison) that compares the extracted answers against the stored community capability specifications. In yet another embodiment, the system may execute a separate, secondary API call to the AI model specifically for the capability comparison—particularly useful for analyzing complex, free-form text answers against nuanced capability descriptions—thereby separating the data extraction task from the logical evaluation task.

The process 500 may involve retry logic at 550 (e.g., up to 5 times, or another count specified by a user or according to a system setting) to ensure successful response generation.

The process 500 includes receiving a response including the community capability mapping results as a structured JSON payload at 555. Based on the response formatting requirements defined in the prompt (e.g., as described regarding 540), this JSON object includes one or more items including, e.g., the question identifier, the answer content (e.g., typed values such as Boolean true/false, text, or date), and citation metadata. The question identifier may link to specific clinical inquiries. The citation metadata may include the page numbers and line coordinates used to support the answer.

The process 500 includes converting the JSON response at 560. This conversion may include automatically parsing and validating the raw output against the internal data structure (or schema) of, e.g., the system 400. This validation ensures that the AI-generated data aligns with the system's standardized fields—such as patient demographics or clinical capabilities—and that data types (e.g., dates, booleans) are formatted correctly for storage in the system's database. The process 500 includes the converted AI-generated results to a database at 565. The queue message is successfully processed and deleted at 570. The process 500 includes displaying the response at 575. For example, the display includes the visual capability indicators (e.g., “Green” for match, “Red” for mismatch) generated by the logic described in FIG. 14, allowing the end user to make an informed decision to Accept or Reject the patient referral at 580.

### II. Data Intake

FIG. 6 illustrates a flow diagram of a data ingestion and standardization pipeline, according to some embodiments. The flow may be executed by the data ingestion engine 410 in FIG. 4. The flow may relate or correspond to 510 and 515 in FIG. 5. To address data heterogeneity across various healthcare platforms and systems using various data structures and formats, the system normalizes all data into internal data structures (or a unified schema) through various methods. As illustrated, the pipeline handles multiple input formats including Extensible Markup Language (XML) 614, Health Level Seven (HL7) 612, and JavaScript Object Notation (JSON) 616.

XML Ingestion. Referral data received in XML format 614 may be processed through a Transport Layer Security (TLS) 1.2+(or higher), authenticated web application post (shown as Web Application 620). Upon receipt, the XML file is stored in a container (e.g., an Azure Blob container) and evaluated by an automated function (e.g., Azure Function) for the presence of attachment download (DL) keys. If attachment keys are detected (630), the system retrieves the corresponding documents (e.g., PDFs, clinical summaries) and stores them in a shared Attachment Blob container. The XML payload is then parsed and converted via the conversion function 640 (applying transformation protocols 642) into the platform's proprietary JSON structure at 650 (map to a JSON package), which serves as the foundation for creating a referral record and linking associated attachments. This path may be the primary normalization route for downstream processing and serves as the template that the HL7 and JSON workflows ultimately follow.

HL7 Ingestion. Referrals submitted in HL7 format 612 are processed through a similar TLS 1.2+ web post mechanism and stored in a format-specific Azure Blob container. For instance, an Azure Function retrieves the HL7 message and first converts it into XML, maintaining all message segments and hierarchical relationships (as defined in the transformation protocols 642 of function 640). Once converted, the XML payload follows the same transformation and attachment evaluation logic as the XML workflow-evaluating for attachment keys (630), retrieving associated files, and mapping to the internal JSON schema at 650. This approach leverages the established XML processing logic, reducing or minimizing redundancy and ensuring consistent normalization across formats.

JSON Ingestion. Referrals originating from JSON-based submissions 616, including those captured through Robotic Process Automation (RPA) integrations, are handled through a distinct endpoint. Although these payloads are already structured, they often vary in schema and field naming. Accordingly, the JSON input is reformatted into a temporary XML representation to align with the platform's standard XML parsing framework (consistent with the supported formats in protocols 642). From there, the XML is processed identically to other XML sources—evaluated for attachments, normalized through static mapping templates, and transformed into the internal JSON schema at 650 used for record creation. This approach ensures uniform handling of all data sources while allowing the RPA channel to operate with minimal friction.

Security and Isolation. As illustrated by the entry point at the web application 620, inbound connections use encryption (e.g., HTTPS/TLS encryption) with tokenized authentication and credential validation to comply with data security requirements (e.g., HIPAA and related security frameworks). Different data formats are segregated by endpoint and container, preventing cross-contamination or schema confusion. Document attachments (e.g., PDFs) are routed to a shared “attachment” blob container linked by metadata references within the corresponding JSON packet.

Output and Utilization. The resulting standardized JSON package 650 may serve as the core data representation for referral packet creation and may be simultaneously persisted in the platform's data layer for downstream analytics, auditability, and machine-learning processes. In certain cases, HL7 data is first transformed to Fast Healthcare Interoperability Resources (FHIR) and then mapped to JSON, simplifying conversion logic and reinforcing schema consistency.

This pipeline provides several technical effects and/or advantages, including, e.g.: (1) Unified multi-format processing where a single system handles XML, HL7, and JSON inputs using adaptive transformation pipelines; (2) Cascading transformation logic (HL7→XML→JSON and XML→JSON) that ensures consistency and reduces mapping complexity; (3) Attachment-aware ingestion (operation 630) that automatically detects and retrieves associated documents during normalization; (4) A proprietary JSON schema that provides consistent internal representation for all referral types; (5) Graceful degradation which allows partial record creation even under field-level mapping errors; (6) Azure-native orchestration that is event-driven and scalable, supporting real-time throughput; and (7) Compliance and isolation via secure, tokenized ingestion paths that maintain HIPAA compliance and tenant data segregation.

Regardless of the input format (HL7, XML, JSON), the system may render the data into a standardized document format (e.g., PDF) to facilitate unified OCR processing. During this intake or extraction phase, the system may apply prompt engineering guardrails to the AI model to prevent over-censorship. For example, the system instructs the model to retain sensitive clinical terms (e.g., regarding wounds or substance abuse) that might otherwise be filtered by standard safety protocols, ensuring the clinical analysis record is complete.

### III. Automated Information Extraction Via Artificial Intelligence

III.a Content Extraction from Referral

The system (e.g., 150, 300B, 400, 3400) is configured to leverage an AI tool, e.g., Azure Open AI, for various prompts where it is provided with clinical records, and provides a responsive analysis based upon the prompt provided. This may include highly structured information (demographics, medications, etc.) as well as custom (customer-defined) responses to unique clinical questions based upon customer needs per location.

### III.a.1. OCR Extraction and Preprocessing

Following the ingestion and normalization of referral data as illustrated in FIG. 5, the system may execute an AI-driven orchestration process to transform unstructured documentation into structured clinical answers. This process may begin with OCR extraction and preprocessing. For example, referral documentation—including clinical notes, consult summaries, and attachments—is first processed using an OCR engine (e.g., Apryse) hosted within an Azure environment. The OCR engine may perform text extraction and/or layout analysis, identifying bounding boxes, text position, and document hierarchy. In addition to geometric coordinates (e.g., X/Y bounding boxes), the OCR process assigns semantic location tags to distinct text chunks. These tags may include, for example, paragraph identifiers (e.g., “Para_001”), line numbers or ranges, word sequence indices, section headers, or page-level section headers. These location tags are preserved as metadata alongside the extracted text and are utilized to create a referenceable index for the downstream AI components. The extracted data is combined into a structured JSON representation, aligning with the platform's internal schema for referral data. Text elements are tagged with coordinate metadata to allow location-based navigation, enabling end users to view extracted data in context within the original document.

### III.a.2. AI Prompting and Orchestration Pipeline

The AI prompting and orchestration pipeline may function to systematically transform the unstructured text extracted from referral documents into structured, query-specific clinical insights. By managing the interaction between the raw data and the AI model, the pipeline may ensure that the generated outputs adhere to certain schema (e.g., pre-defined schema) and accurately address both standard and customer-defined clinical questions, rather than merely providing unstructured summaries.

AI interactions may be executed through an AI model, e.g., Azure Functions, that orchestrates data transfer, prompt construction, and response handling with the Azure OpenAI Service (e.g., GPT-4.1 Mini). In this configuration, the AI model functions as a generative inference engine defined primarily by its input-output interface, abstracting the specific internal architecture (e.g., neural network depth or attention mechanisms). The system treats the model as a transformation layer that converts a specific composite input into a formatted output. Documents exceeding the model's token limitations may be semantically segmented into smaller chunks, preserving contextual relevance and minimizing fragmentation. When the content (including, e.g., unstructured text extracted from the referral documents, segmented to fit token constraints if needed) is prepared, the system initiates the inference process by constructing and transmitting an API call to the AI service.

The API call may include a structured prompt payload. The prompt may serve as the directive logic that guides the AI model to selectively extract and format specific data points from the prepared content. For example, an API call includes: (i) a primary instruction set (defining core extraction rules and schema); (ii) a customer-defined question set (comprising custom inquiries and categories); (iii) facility-specific capability specifications, (iv) question metadata (specifying data types such as text, date, choice, multi-choice, etc.); and (v) response formatting requirements (defining the JSON structure including patient identifiers, record IDs, question IDs, and typed answers). The facility-specific capability specifications may include, e.g., acceptance criteria, required answer formats, and logic for determination. For instance, the prompt may include a rule stating “If BMI>35, mark compatibility as ‘Fail’”. This allows the AI model to not only extract the value (e.g., “BMI: 38”) but also generate a preliminary compatibility indicator or recommendation (e.g., “Status: Fail”) directly within the response payload based on the provided constraints.

The prompt payload construction may involve passing the OCR-generated location tags (e.g., paragraph IDs or sequence numbers) to the AI model alongside the text content. The AI model is explicitly instructed to cite these location tags in its structured response when identifying the source of an answer. For example, if the model identifies a diagnosis of “Hypertension” in the text block labeled “Para_12”, the returned JSON object for that question includes a reference to “Para_12”. This enables the system to map the AI-generated answer back to the specific text chunk and its corresponding geometric coordinates on the document image, facilitating the precise visual indication described in, e.g., Section III.b.

In some embodiments, the prompt payload may explicitly provide a set of valid answer options for each query (e.g., [“Yes”, “No” ], [“Independent”, “Assist x1”, “Dependent” ]). These options are provided to the machine learning component to constrain the generated output to a specific format that aligns with the node's capability specifications. For example, if a facility's capability rule is defined as accepting “Independent” or “Assist x1” but rejecting “Dependent,” the AI model is instructed to select the answer solely from that specific options list. This ensures that the resulting compatibility indicator (e.g., Pass/Fail) is derived from a standardized data value that directly maps to the facility's internal logic.

From processing the payload, the model returns output in a JSON format, which is automatically validated and ingested into the platform's PostgreSQL database. Extracted results are simultaneously rendered in the application UI and stored as JSON for traceability. Prompt-response pairs may be archived for auditing and compliance, including timestamps, model parameters, and associated referral identifiers.

### III.a.3 Standard and Custom AI Prompts

The system includes a set of standard prompts-baseline prompt templates that extract standardized healthcare data, uniform across all customer organizations. Examples of standard prompts include:
Prompt 1—Large File Chunking (Internal): Handles oversized input files through segmented processing (see Example 2).
Prompt 2—General Information: Extracts demographic and patient-identifying data (see Example 3).
Prompt 3—Medication Information: Identifies prescriptions and dosages (see Example 4).
Prompt 4—Diagnosis and History: Extracts ICD codes, diagnoses, and relevant historical data (see Example 5).

Alternatively or additionally, customers may define custom (customer-defined) prompts (or referred to as “clinical prompts”) for extracting data relevant to their operational, clinical, or appropriateness-assessment workflows. Such questions may be stored in the platform's metadata layer with unique identifiers, associated category tags, and data-type constraints. Custom prompt execution may occur through, e.g.:
Prompt 5—Large File Chunking (Customer Defined) (see Example 6).
Prompt 6—Clinical Prompt (Customer-Specific Extraction) (see Example 7).

These prompts may allow organizations to tailor information retrieval without modifying the core AI framework, supporting high configurability across organizations, facilities, or locations.

### III.a.4 Customer-Defined AI Form Population and Dynamic Prompt-Oriented Clinical Data Structuring System (AI Clinical Answers)

In some embodiments, the system includes a configurable artificial intelligence (AI) system configured to automatically populate organization-defined clinical review forms using structured data derived from unstructured referral documentation. This system enables healthcare organizations to configure custom question sets, manage how those questions are included in AI processing, and receive structured, explainable, and auditable answers generated by a generative AI model (e.g., a Large Language Model or LLM). The solution dynamically merges organization-specific configurations with a platform-level master prompt schema to generate precise, context-aware responses, ensuring clinical transparency, operational efficiency, and compliance with data security standards.

System Architecture Overview: The AI form population system may operate (partially or entirely) within a multi-tenant cloud computing environment (e.g., Microsoft Azure), leveraging various components such as:
(i) Serverless Compute Functions (e.g., Azure Functions) for parallel AI processing;
(ii) AI Inference Services (e.g., Azure Foundry and OpenAI GPT-4.1-mini) for generating responses;
(iii) Structured Databases (e.g., PostgreSQL) for structured and JSON-based data storage; and
(iv) Object Storage (e.g., Azure Blob Storage) for intermediate document caching and metadata management.

Data—including referral text, AI prompts, and responses—is encrypted both in transit and at rest. Each tenant (organization or facility) operates within a logically segregated environment, ensuring data isolation and compliance with regulatory standards (e.g., HIPAA) regarding the handling of protected health information (PHI).

Organizational Configuration and Question Definition: A tenant (organization) defines custom clinical review questions via a configurable administrative interface. These questions are grouped into categories, which can be assigned selectively to one or more facilities (termed “communities”) within the organization. This enables granular control—for example, a facility that does not admit ventilator-dependent patients may have the “Ventilator” category unassigned. A question record may contain: (i) question text (string field); (ii) answer type (string, date, single choice, or multi-choice); (iii) category association; (iv) metadata (facility assignment, display order, and visibility); (v) a prompt inclusion flag (determining whether to include the question in ai processing); (vi) a unique identifier for schema mapping, or the like, or a combination thereof.

Questions are stored in a hybrid relational/JSON schema, allowing structured querying alongside flexible category-based organization. Organizations can independently define which questions are AI-populated and which are manually entered by users, enabling tailored configurations per operational or compliance requirements.

Master Prompt Composition and AI Orchestration: A master prompt template (e.g., stored as an appendix or configuration file) resides at the platform level, defining, e.g.: (i) contextual boundaries for the model (e.g., “Only use content found in referral text”); (ii) output structure requirements (e.g., an enforced JSON schema); (iii) instructions for precision, traceability, and compliance, or the like, or a combination thereof.

When a referral is processed, the system dynamically merges, e.g.: (i) The master prompt; (ii) The organization's selected question set; and (iii) The extracted referral data and positional information (e.g., paragraph-level metadata for text, to facilitate traceability and intra-document navigation as described elsewhere herein) into a single composite AI request. This composite payload is transmitted securely to the LLM endpoint through the orchestration layer (e.g., Azure Functions), which supports parallelized execution for concurrent referral processing. Large referral documents may be chunked into smaller semantic segments to preserve context within token limitations, ensuring consistent inference accuracy across complex documents. A redundant AI execution pipeline may be included to provide failover handling: if a timeout or model error occurs, the system automatically retries using a backup processing channel.

AI Processing, Validation, and Structured Output: The AI model receives the composite prompt and returns a structured response (e.g., JSON) containing: (i) mappings of each question identifier to its corresponding answer; (ii) metadata fields (including, e.g., confidence score, timestamp, model version); and (iii) compliance flags indicating adherence to required formats.

Upon receipt, the system performs schema validation to ensure type conformity (e.g., date, array, text). If the output fails validation, the system retries automatically. If the second attempt fails, a message is surfaced to the user indicating which questions require manual completion. The structured results are then written to the database with strict separation between AI-generated answers and user-edited answers. A field is linked to its originating referral and question ID, enabling traceable one-to-one mapping across all data layers.

User Interface and Review Workflow: Within the application's clinical review interface, AI-generated fields are presented alongside manually entered fields. AI-derived fields are visually denoted (e.g., with a green wand icon), signaling that the content was system-generated. Users can: (i) Review and edit AI-generated answers directly; (ii) Re-run AI generation for the entire form if needed (e.g., after document updates); and (iii) Submit manual overrides, which are recorded as distinct versions from the AI-generated response. While only the most recent value is displayed in the interface, the system maintains both versions for audit and model-improvement analysis. A user modification is flagged in the database, allowing the platform to track and analyze where human correction occurs (and frequency) to refine future AI accuracy.

Data Management, Auditability, and Compliance: Prompts, responses, and edits are logged with, e.g.: (i) timestamps; (ii) model version; (iii) execution ID; and (iv) organization and user identifiers. This log allows complete reconstruction of an AI output, including its source prompt, referral data, and question configuration at the time of inference. Prompts and responses are encrypted and version-controlled, ensuring clinical and regulatory traceability for auditing and compliance validation. Additionally or alternatively, if an AI process is manually re-run, both the original and subsequent responses are retained. Failed AI attempts and fallback operations are logged for system diagnostics. This architecture creates a transparent, audit-ready framework for clinical documentation automation.

Security and Data Isolation: An AI processing occurs within a private cloud deployment (e.g., Azure Foundry) using AI endpoints with no external data transmission. PHI remains entirely contained within the secure tenant boundary. The multi-tenant design employs database-level partitioning and role-based access control (RBAC) to ensure that each organization can access only its own data. Exemplary encryption standards include, e.g., AES-256 encryption at rest and TLS 1.2+ encryption in transit.

An exemplary system workflow includes:
(a) Referral Intake: The system receives referral data and extracts text, structured metadata, and paragraph references.
(b) Configuration Lookup: The system retrieves the organization's question set and AI inclusion flags.
(c) Prompt Assembly: The system merges the extracted content, selected questions, and master prompt schema.
(d) AI Execution: The system sends payload to the cloud-hosted LLM; processes concurrently using serverless functions (e.g., Azure Functions).
(e) Validation: Parses and validates the returned JSON for completeness and schema adherence.
(f) Storage: Writes validated responses to AI and user-edit columns in the database.
(g) Display and Interaction: Presents AI-populated fields in UI with transparency icons; allows edits and reruns.
(h) Logging and Auditing: Captures all prompt configurations, outputs, and user interactions for compliance and performance optimization.

The disclosed configuration offers several technical effects and/or advantages including, e.g.:
(a) Configurable AI Schema Integration: Enables each organization to define its own question sets and determine which fields are AI-driven versus manually populated.
(b) Dynamic Prompt Assembly: Combines master prompt logic with tenant-specific questions and extracted referral text at runtime to produce customized LLM inferences.
(c) Parallelized Cloud Processing: Executes multiple AI jobs simultaneously using serverless functions for scalable, high-volume referral processing.
(d) Schema Validation and Self-Healing Retry: Automatically enforces type conformity, retries failed validations, and flags incomplete outputs for manual review.
(e) Versioned Human-AI Collaboration: Tracks and stores both AI and human-modified responses for auditability, explainability, and model refinement.
(f) Secure, Multi-Tenant Architecture: Maintains strict data isolation, encryption, and PHI containment within the private cloud boundary, ensuring compliance.
(g) Fully Auditable AI Provenance: Logs every inference with complete prompt lineage, model version, and user context, ensuring transparent traceability.
(h) Customizable Clinical Logic: Supports organization-level configuration for facility-level workflows, ensuring AI documentation aligns with local care requirements.

FIG. 7 illustrates an exemplary AI custom-answer workflow, according to some embodiments. The workflow 700 shows a dynamic prompt assembly and execution process utilized by the AI form population system.

The process 700 begins at 710, where the system performs referral generation and data extraction processes. As described elsewhere in the present document, this involves ingesting the referral documentation and extracting unstructured text and metadata (e.g., via OCR).

At 720, the process 700 includes determining the facility associated with the referral. For multi-tenant and multi-facility configurations, this operation identifies which specific configuration rules and question sets apply to the current case.

At 730, the process 700 includes assembling the AI prompt. This operation functions as a dynamic merger of data, combining the specific facility context determined at 720 with inputs from two distinct configuration sources:

At 740, the process 700 includes obtaining a master prompt architecture, e.g., a platform-level template that defines the structural rules, JSON output schema, and contextual boundaries for the AI model.

At 750, the process 700 includes accessing a question library, e.g., an organization-defined repository of clinical questions specific to the facility (e.g., “Ventilator status,” “Mobility needs”).

Once assembled at 730, the composite payload is transmitted at 760 as a prompt to the LLM layer (e.g., Azure OpenAI). This operation may involve parallelized execution via serverless functions to ensure scalability.

At 770, the process 700 includes performing user validation via AI (and/or schema validation). This operation involves parsing the returned JSON structure for conformity and presenting the AI-generated answers to the user interface for review, where users can validate or edit the system's findings.

At 780, the process 700 includes performing logging. This ensures that the exact prompt configuration, model version, and execution timestamp are archived for auditability and compliance. The logging may be performed before, simultaneously, or subsequent to 770.

FIG. 8 illustrates a diagram for LLM clinical question answering, according to some embodiments of the present technology. The flow 800 shows an exemplary operational flow where these prompts drive the clinical question answering process. The flow 800 begins with a referral packet and other referral information 810 being processed through optical character recognition (OCR) 820 to extract text content.

The system maintains a database of question prompts 830 that are tailored for specific types of analyses. These prompts are combined with the extracted text through a query generation process 840 that structures the input for the AI model (e.g., LLM). Each query includes instructions for the AI model to act as a referral reviewer, along with specific questions and contextual parameters. In some embodiments, the query generation at 840 may be implemented as described in FIG. 7 including utilizing one or more of the facility determination 720, the prompt assembly 730, the master prompt architecture 740, and the question library 750.

An AI component (e.g., LLM component) 850 processes the query against the extracted text. The LLM performs two main functions: identifying contextually relevant text segments and generating structured answers to specific questions. For text identification, the model locates words or sections that match, are synonymous with, or are semantically relevant to specified keywords. For question answering, the model generates responses following specified answer formats and types.

The output 860 includes structured answers that conform to specified formats (e.g., selections from predefined options) and identified text segments for highlighting. These outputs are provided for user review and can be modified or supplemented by human reviewers.

The flow 800 implements both contextual text identification for user review assistance and automated question answering while maintaining consistent formatting and structure in the outputs.

Merely by way of example, a community may insert a question into the system to ascertain a patient's past medical history and the prompt provided to the AI model may be:
Summarize the details of the patient. Here are the specific aspects to include in your summary:
   Primary referral reason/diagnosis
   Recent medical issues
   Current medications
   Past medical history (PMHx) with a focus on items that may impact current care needs
   Any recent tests or procedures
   Important symptoms or findings
Ensure that your summary focuses on the most critical information necessary for the patient's ongoing care, omitting any identifying details such as the patient's name or birth date and do not provide treatment recommendations or considerations.”
Prompts may indicate that specific answer options are available and need specific answer formatting (e.g., numeric, dichotomous [yes/no], multi- or single select multiple choice question, etc.
Prompts may also include instructions to indicate if there was not enough information to answer the question. The prompt may indicate to include in the answer field that there was not enough information, or was otherwise unable, to properly answer the question. The prompt may instead indicate to leave the answer blank in such cases. The prompt may indicate to set a logic flag to true if not enough information is available to answer the question, which can be used to display an icon indicating that the LLM was unable to answer the question.

FIG. 9A shows an exemplary LLM answer output 900A in response to prompt and provided referral information, according to some embodiments of the present technology. As shown, the system presents a summarized narrative (e.g., “The patient is a 43-year-old male presenting with ischemic stroke . . . ”) generated based on the specific prompt instructions described above, to focus on care information of interest while omitting identifiers.

FIG. 9B illustrates example clinical questions and answers 900B including icons indicating LLM answered, and LLM answered/user edited, according to some embodiments of the present technology. Questions that are answered by the LLM may be indicated with a specific icon, as shown in the figure (e.g., a “wand” icon). Other icon(s) may be shown to indicate that the LLM generated answer has been human reviewed and/or edited. For example, the following icons may be used. A first icon may indicate the answer is LLM generated. A second icon may indicate the answer was LLM generated but human edited/reviewed (e.g., an “edit” pencil icon). A third icon (or absence of an icon) may indicate that an LLM was unable to answer. Similarly, the LLM may be prompted to either fill in the answer indicating that an answer, or a portion of answer, was unable to be found. Alternatively, it may simply leave the answer field blank. The prompt includes instructions for the LLM on how to handle unanswerable questions.

### III.a.5 Retrieval-Augmented Generation (RAG) Integration

While the extraction pipeline described in subsections III.a.2, III.a.3, and III.a-4, functions to extract information, e.g., clinical facts, from the referral data package, the system is configured to correlate these facts with real-world operational constraints to determine admission suitability. For advanced contextual retrieval, the system employs retrieval-augmented generation (RAG) using an AI tool, e.g., Azure AI Search. This allows the model to query organization-specific truth tables and knowledge bases that define, amongst other items, e.g., facility capacities, acceptance criteria, medication cost, payor contracts. E.g., 541 and 542 in FIG. 5. By referencing these “ground truth” data sources, the system ensures that clinical decisions are based on current facility capabilities rather than static training data.

(i) Facility Capabilities: describes facility capabilities across multiple attributes (wheelchair accessibility, bariatric capabilities, ventilator criteria, etc.).

Examples: Criteria for Organization A, Facility 1 may list multiple high flow oxygen settings and equipment that can be accepted, as well as those that need to be denied. The correlation between this and the referral data determines if this referral can be accepted or not (visualized via indicators, criteria scoring, and matching screens). For instance, the model can evaluate other facilities within that organization to determine if another facility is more appropriate (i.e., has more “yes” matches on criteria).

(ii) Acceptance Criteria: Hard yes/no constraints that determine if a referral can be accepted.

Example: Criteria for Organization A, Facility 1 may list BMI>30 as “No”. If a referral is received where a patient has a BMI>30 (extracted via AI or data mapping), the system prompts users through visual indicators to deny the patient. Concurrently, similar to Facility Capabilities, the system may evaluate other facilities within the organization to determine appropriateness at other sites.

(iii) Medication Costs: Price lookups and dosing norms to estimate daily/weekly cost.

Example: Medication cost databases may be organization-specific and reflect their unique organizational formulary. Organizations without a formulary may leverage the CMS average wholesale price data. This data is correlated with the medication list extracted from the referral to estimate daily, weekly, and monthly medication spend.

(iv) Payor Contracts: List payor contracts and correlative information (levels, plans, start/stop dates, terms, rates, etc.).

Example: If a referral is received for Anthem Blue Cross Blue Shield, the LLM/RAG evaluates details of this payor and correlates them with patient descriptors (diagnosis, care notes) to determine which plan level will likely be billed, as well as reflecting authorization notes and contract details.

By augmenting LLM input with structured retrieval data, the system may enhance the accuracy and relevance of extractions without relying on global datasets. This may facilitate tenant-level data isolation and personalization.

FIG. 10 illustrates an architecture diagram for retrieval-augmented generation (RAG) integration, according to some embodiments. This diagram illustrates how these data sources are integrated into the decision-making workflow of an online communication matching flow executed during a runtime request. The architecture 1000 enables the system to generate specific and accurate clinical responses by grounding the AI model in organization-specific data rather than relying solely on pre-trained global knowledge.

As illustrated, the architecture includes a web app 1020 (e.g., user-facing clinical interface) that transmits requests to an app server/orchestrator 1030 (e.g., the Azure Functions). The orchestrator 1030 acts as a central logic hub, managing the flow of data between the knowledge retrieval components and the generative AI components. It may be implemented as a serverless compute component within the system 400, corresponding to the queue trigger and payload generation logic shown at 530, 540, and 545 in FIG. 5, or the prompt assembly logic 730/840 shown in FIGS. 7 and 8.

To process a request, the orchestrator 1030 first executes a “Query” to an AI search service 1040 (e.g., Azure AI search service). The service 1040 locates and retrieves specific information regarding an organization (or community) from the data sources 1060 (e.g., corresponding to 544 in FIG. 5), which serve as the “ground truth” for an organization (or community). As shown, these data sources may include:
Complex Scoring Rules: Logic for calculating risk or admission suitability (e.g., LACE scores, PDPM).
Capabilities Database: Structured SQL records defining what a specific facility can or cannot handle (e.g., “Ventilator: Yes,” “Bariatric: No”).
Other Sources: Additional unstructured or semi-structured documentation relevant to the tenant.

The AI search service 1040 retrieves the relevant “Knowledge” (e.g., the specific capability rules for the target facility) and returns it to the orchestrator 1030. The orchestrator 1030 then generates a composite payload (or “augmented payload” or “combined payload” described with respect to 545). This payload combines the original clinical inquiry (“Prompt”) (e.g., corresponding to the query generated at 740 in FIG. 7 or 840 in FIG. 8) with the retrieved organizational (or community-specific) facts (“Knowledge”).

This augmented payload (“Prompt+Knowledge”) is transmitted to a generative AI service 1050 (e.g., the Azure OpenAI GPT-4 or ChatGPT). The service 1050 performs a generative inference process based on the provided context. By analyzing the “Prompt+Knowledge,” the service 1050 generates a structured “Response” based on the information retrieved from the referral that is factually aligned with the organization's (or community's) real-world capabilities. This response is returned to the orchestrator 1030 for subsequent use.

By augmenting the AI model input with this structured retrieval data, the system enhances the accuracy and relevance of extractions and decisions without relying on global datasets. Furthermore, because the data sources 1060 are indexed specifically for each organization, this architecture ensures tenant-level data isolation and personalization, preventing data leakage between different healthcare organizations using the platform

The structured response generated by this flow may serve one or more functions within the platform. For example, it enables visual presentation at the web app 1020, where the structured JSON data (e.g., boolean values indicating capability matches) is rendered as visual indicators (e.g., color-coded “Green/Red” status badges), allowing the user to rapidly assess suitability. As another example, the structured response generated by this flow may guide the visualization of at least a portion of the extracted answers for user review, adjustment, or confirmation. As a further example, the structured response drives the system's automated community search logic.

FIG. 11 illustrates a workflow 1100 for providing answers to clinical questions along with specific citations to referral information, according to some embodiments. This workflow 1100 illustrates an internal processing logic utilized by the Azure AI Search service (1040) and Generative AI (1050) described above in FIG. 10, specifically illustrating the vector-based retrieval mechanism.

The process 1100 begins with the ingestion of a referral packet and other referral information at 1110 being processed through OCR and chunking at 1120, which converts documents to text and divides them into smaller, semantically distinct segments.

The chunked text is processed by an embedding model 1130 that converts text segments into numerical vectors. These vector representations are stored in a vector store index 1150, creating a searchable database of text chunk embeddings. This index may correspond to the “Data Sources” (1060) queried in the high-level architecture.

When a query 1140 (e.g., the “Query” shown in FIG. 10) is received, it undergoes two parallel processes. In one process, the query 1140 is passed directly to the Large Language Model (LLM) 1190 to frame the context for response generation.

Along the other process, the query 1140 is input to the embedding model 1130 to be converted into a vector representation (query embedding) 1160. The system performs pairwise correlation calculations 1170 between the query embedding and stored chunk embeddings to evaluate or identify matching (e.g., evaluating semantic similarity between a specific clinical question and the facility's capability rules). A ranking and retrieval component 1180 selects the most pertinent text chunks based on these calculations.

The LLM 1190 receives both the original query and the retrieved relevant text chunks as context (forming the “Augmented Payload” described in FIG. 10). It generates two outputs including citations 1192 that link answers to specific sections of the source documents and answers 1194 to the query based on the provided context.

### III.a.6 Post-Processing, Validation, and Monitoring

Extracted information is automatically parsed and validated before storage (see, e.g., 565 and 570 in FIG. 5). The system performs schema and field-type validation to ensure compatibility with internal data structures. A user-facing field may be displayed alongside its source location reference within the original document (see, e.g., 575 in FIG. 5 and “Document-to-AI Positional Correlation and Visual Highlighting System” section below), allowing users to cross-verify extracted data visually. In some embodiments, an icon may be presented in a user interface to indicate that a user has modified the AI output after generation.

After ingestion, user edits are monitored to detect discrepancies between AI responses and manual corrections. These events may be logged to support continuous quality improvement and refinement of prompt performance over time.

In the exemplary implementation, the entire system operates within the Azure cloud environment in a multi-tenant configuration, leveraging:
(i) Azure Functions for orchestration and prompt execution;
(ii) Azure Blob Storage for OCR outputs and extracted content caching;
(iii) Azure AI Search for RAG queries; and
(iv) PostgreSQL databases for structured record storage and metadata management.

Relevant communication may occur via secure HTTPS/TLS channels with tokenized authentication, ensuring HIPAA-compliant data handling.

The system provides several technical effects and/or advantages including, e.g.:

Hybrid AI-Orchestrated Content Extraction: Combines OCR layout intelligence with LLM-based semantic analysis to produce structured, location-aware data from unstructured referral content.

Structured Prompt and Response Architecture: Implements bidirectional JSON schema exchange, enabling standardized model communication and seamless database ingestion.

Customer-Specific Customization: Allows each organization to define and manage its own extraction schema, question categories, and data formats-without retraining models.

RAG-Enhanced Precision: Uses Azure AI Search to inject organization-specific (or community-specific) data into prompts, improving contextual accuracy and ensuring tenant isolation.

Semantic Chunking for Context Retention: Performs intelligent segmentation of long documents to preserve narrative coherence within token constraints.

Auditability and Traceability: Archives every prompt, response, and data transformation event, supporting explainability, compliance, and reproducibility.

Integrated Azure Architecture: Executes within an event-driven, serverless framework optimized for scalability, cost efficiency, and parallel task orchestration.

III.b Document-to-AI Positional Correlation and Visual System

In some embodiments, visual indicators may be provided to facilitate auditability, user navigation of information, or the like, or a combination thereof. For instance, AI-generated answers may be visually mapped to their corresponding regions within source documents, thereby enhancing transparency, auditability, and interpretability of large language model (LLM) outputs in healthcare referral processing. Merely by way of example, boxes are drawn around sections of the source document (e.g., a PDF) to highlight supporting evidence for an AI response and ease user navigation.

This feature allows end users to see exactly where within a patient's referral document an AI model extracted its information, achieved by leveraging OCR-derived metadata and coordinate-based bounding box visualization. The system may dynamically generate these bounding boxes in a document viewer interface (e.g., a ReactJS-based PDF viewer), enabling an interactive, explainable AI interface.

FIG. 12 illustrates a flow diagram 1200 for a document-to-AI positional correlation and visual highlighting system, according to some embodiments. In some embodiments, this system provides visual indicators to facilitate auditability and user navigation. For instance, AI-generated answers are visually mapped to their corresponding regions within source documents, thereby enhancing transparency and interpretability of AI model (e.g., LLM) outputs. The process 1200 may be implemented in system 150, 300B, 400, or 3400.

### III.b.1 Text Extraction and Coordinate Capture

The process 1200 begins with document ingestion and analysis, as illustrated by operations 1210 through 1230 of FIG. 12. At 1210, a source document (e.g., a PDF) is received. At 1220, in a text extraction phase, text and coordinate metadata are extracted and stored. A referral document undergoes processing through an OCR engine (e.g., Apryse), which extracts text as discrete lines along with metadata for page number, line number, and positional coordinates (X/Y values).

At 1230, these details are persisted in the application's PostgreSQL database, associating each paragraph or line with its geometric location within the source document (e.g., PDF). In some embodiments, the generated coordinate map includes a multi-layer (e.g., dual-layer) location schema. A first layer includes geometric coordinates (e.g., absolute X/Y bounding box values and dimensions) utilized specifically for rendering visual overlays, such as drawing highlight boxes around text on a canvas. A second layer includes semantic location tags-such as paragraph identifiers (e.g., “Para_001”), page numbers, line ranges, or word sequence indices. These tags provide a logical reference handle for the extracted text. While the geometric coordinates are used for the visual display, the semantic location tags are utilized by the system (e.g., ML engine 430) to associate relevant text chunks with specific queries and compatibility indicators. This allows the system to link an AI answer to “Paragraph 5” logically, and then look up the geometric coordinates of “Paragraph 5” to visually highlight it.

This creates a dual coordinate storage schema supporting both geometric (absolute X/Y) mapping and semantic (row index range) mapping. When OCR quality is degraded or the AI response lacks positional precision, the system can alternatively map content by row index ranges (e.g., “lines 16-20”) to ensure consistent traceability across varying document qualities. A text unit (e.g., paragraph or line entity) is linked to a unique document ID and stored in a normalized format, enabling downstream cross-referencing by AI models and visualization components.

### III.b.2 Chunk Generation and Prompt Structuring

Following extraction, the system proceeds to chunking and prompting, corresponding to operation 1240 (AI Model Processing) of FIG. 12. During the chunking and prompt configuration phase, content is segmented with embedded positional data. OCR-extracted content is aggregated into text chunks, e.g., not exceeding 80,000 characters, to meet token constraints for the AI model. A chunk may include the embedded positional metadata for every line or paragraph, allowing the model to interpret and return references to specific regions of the source document.

In some embodiments, the AI prompt includes two distinct components:
Instructional context—describing how to interpret the embedded metadata and how to format the output.
Customer-defined clinical questions—defining the queries for which the model needs to extract relevant content.

In an exemplary implementation, the model is explicitly instructed to include page, line, and/or coordinate references for every extracted answer, enabling subsequent spatial correlation.

### III.b.3 Model Processing and Response Mapping

The AI inference phase generates the coordinate data (1150) and parses the response (1160). During AI inference (e.g., via Azure OpenAI GPT-4.1 Mini), the model receives the text chunk, questions, and instructions to return both answers and positional references. A text chunk is processed by the AI model (e.g., GPT-4.1 Mini) via an orchestration layer or tool (e.g., Azure Functions), which handles orchestration and ensures schema consistency. The model output captured as bounding box coordinates at 1250 may represent the raw spatial data identifying where the answers were found in the document.

AI response parsing occurs at 1260. The model returns responses in a structured JSON format, which includes items such as the question identifier, the answer content, and a list of page-line or coordinate pairs used to support that answer. For large documents processed in multiple chunks, results may be aggregated and reconciled into a single unified response. This response retains at least a portion of or all positional metadata, ensuring each of at least a portion of AI-derived answers is linked back to its physical document source.

### III.b.4 Bounding Box Rendering and Interaction

Visual rendering occurs when the application receives, at 1270, the coordinate data (from 1250) and displays, at 1280, the visual overlays. The react application involved at 1270 (e.g., a ReactJS-based PDF viewer) may serve as the user interface that receives the bounding box coordinates generated at 1250.

At 1280, the application's viewer dynamically renders bounding boxes around the text regions identified in the AI response. For bounding box rendering, the viewer displays dynamic highlight overlays referencing stored coordinates.

Bounding boxes are drawn:
Using absolute X/Y coordinates when precise positional data exists, or
By row range highlighting when coordinate accuracy is unavailable.

Each bounding box is persisted in the database with its coordinates and the unique AI response/question ID.

This interactive capability creates a bidirectional link between the visual document view and the structured AI output. Users can hover or click on a visual element—either the bounding box on the document image or the corresponding clinical question/answer in the analysis panel—to view an informational tooltip. For instance, in embodiments where a single referral is being evaluated against multiple potential service providing nodes (e.g., multiple facilities), this tooltip is configured to display a multi-facility compatibility breakdown. For example, hovering over a specific answer (e.g., “Ventilator Required: Yes”) triggers a popup listing the compatibility status for each candidate facility regarding that specific data point. The list may utilize visual indicators (e.g., color codes) to show which facilities are compatible (e.g., Green), incompatible (e.g., Red), or require follow-up (e.g., Yellow). This allows the reviewer to instantly assess how a single clinical fact impacts the eligibility of the referral across the entire network of candidate nodes without navigating away from the current view.

### III.b.5 Persistence, Audit, and Compliance

For audit logging, the bounding box, coordinates, and response mappings are stored for traceability and regulatory review. Bounding box data—including coordinates, associated question IDs, and AI response references—are stored in the PostgreSQL database for audit and compliance purposes. This may ensure that each AI-derived result can be traced back to the exact location within the source document, the specific prompt-response pair that generated it, or both.

These records form part of the system's overall audit log, supporting regulatory transparency, model validation, and clinical review processes.

### III.b.6 Automated Review Aids: Keyword and Contextual Highlighting

To facilitate user review, the system processes the referral packet to visually indicate (e.g., by highlight, bounding box) specific keywords, as illustrated at 1280 discussed above. A keyword may be associated with a category corresponding to a category of the clinical review (e.g., “Cardiology,” “Mobility”). The visually indicated keywords allow the reviewing user to easily navigate the document to identify terms related to a specific question or category, or to navigate to specific keywords one at a time.

The visually indicated keywords can be selectively filtered using a “word bank” interface depending on the active clinical review section. The keywords in the word bank are organized by keyword categories. One or more categories and words may be provided by the system. One or more categories and words may be manually customized and curated by the organization or community users. The system employs fuzzy matching logic to match keywords directly with words in the referral packet text, ensuring identification even if minor OCR discrepancies exist.

In some embodiments, an AI-implemented process (e.g., an LLM or NLP process) is utilized to identify words or sections that are contextually relevant to the keywords, even if they are not exact matches. For example, the system passes a prompt along with text from the referral packet to the LLM; this prompt includes instructions to identify and return a list of words or segmented text chunks that are synonymous with, or semantically relevant to, a list of keywords for a certain category.

The returned list of words and text chunks are then visually indicated (e.g., highlighted) in the referral packet for user review. This functionality visually indicates (e.g., highlights) relevant text related to a category that may be missed by simple keyword matching, effectively identifying or indicating logically relevant sections based on the semantic context (e.g., highlighting “shortness of breath” for a “Respiratory” category even if the specific keyword was not in the dictionary).

The system exhibits one or more technical effects and/or benefits including:
Explainable AI in Document Context—Links AI responses to exact document regions, providing traceable, human-verifiable results that enhance interpretability in healthcare decision workflows.
Dual Coordinate Storage Schema—Supports both geometric (X/Y) and semantic (line range) mapping, allowing resilience across varying OCR quality and document fidelity.
Dynamic Visualization—Generates real-time bounding boxes in a web-based PDF viewer, improving user understanding of AI-derived answers.
Interactive Bidirectional Navigation—Users can click or hover to reveal question—answer associations, promoting transparency and confidence in automated extraction results.
Persistent Audit Trail—Stores positional data, AI outputs, and coordinates together in a normalized structure for future validation and compliance verification.
Modular Azure-Orchestrated Design—Integrates tightly with Azure Functions and PostgreSQL, providing scalable, event-driven orchestration and consistent schema governance.

For example, an AI generated answer to the question “Does the patient have any documented history of medication non-compliance?” may navigate the patient to sections within the document referencing where that specific information was found, with a colored bounding box around the section (it may include specific physician notes, rehabilitation notes, EMAR admin findings, etc).

FIGS. 13A and 13B illustrate example user interfaces 1300A and 1300B displaying the visual mapping, where AI-generated answers are linked to their corresponding regions within a source document. The application's ReactJS PDF viewer dynamically renders bounding boxes around the text regions identified in the AI response. The bounding boxes may be visually (e.g., color) coordinated to match whether the corresponding response is determined to be compatible, incompatible, or requiring follow-up (e.g., green, red, and yellow, respectively) based on compatibility with a particular facilities capabilities.

### III.c Interactive Context Linking and Document Navigation System (or Referred to as Intra-Document Navigation)

In some embodiments, a method is provided for interactive context navigation, enabling users to directly access the precise sections of a document that supports a given AI-generated answer. This feature is tightly integrated with the bounding box and coordinate mapping system, forming a bidirectional linkage between structured AI outputs and their unstructured document origins. The system utilizes the location tags returned by the AI to query the coordinate map. For example, if the AI response cites “Para_05”, the system retrieves the bounding box coordinates associated with “Para_05” from the database to render the visual overlay. This creates a robust linkage where the AI reasons using semantic tags, and the UI renders using geometric coordinates. The system enhances transparency, verification, and explainability by allowing users to instantly locate the evidence used by the AI model in generating its responses.

When a user reviews AI-populated responses within the application's questionnaire interface, each answer is accompanied by a navigation icon (e.g., a wand symbol). This icon represents an interactive link between the AI response and one or more supporting regions within the original document.

Upon selecting the icon, the application automatically scrolls and highlights the relevant section(s) of the document displayed in the document viewer (e.g., ReactJS PDF viewer). The system uses preloaded positional metadata—captured and stored during the OCR and bounding box process—to identify exact text coordinates or paragraph rows.

The navigation process is instantaneous, providing the user with a contextual understanding of how the AI derived its response without requiring manual searching or external referencing.

### III.c.1 Data Structure and Metadata Linking

An AI response record includes a set of coordinate references (page, X/Y bounds, and/or line ranges) and/or OCR-derived location metadata tags (e.g., paragraph IDs) corresponding to one or more document regions that informed the model's answer. These references are persisted in a crosswalk table within the system database (e.g., a PostgreSQL database), associating:
AI response ID
Question ID
Document ID
Page number(s)
Coordinate data (X/Y or line range)
Region sequence number
This linking is performed at the individual question level. Each specific clinical question (e.g., “Does patient have dementia?”) is directly associated with the specific text chunk(s) and coordinates used to answer it, allowing for precise, question-specific verification rather than just a document-level association. This structure supports multi-region mapping, allowing a single AI-generated answer to link to multiple non-contiguous regions within the document. The number of distinct mapped regions is displayed next to the navigation icon, enabling users to cycle through each supporting section using up/down arrow controls.

### III.c.2 Navigation Execution and Client-Side Rendering

A navigation action is executed on the client side within the application (e.g., ReactJS application). When a user selects the navigation icon:

The application retrieves the preloaded coordinate data for the selected AI answer. It auto-scrolls the PDF viewer to the corresponding page and viewport. A visual indication, e.g., a bounding box or region, is dynamically rendered. The visual indication may be implemented as, e.g., a colored outline, around the source text. This outline may be achieved using a transient highlight layer drawn over the document canvas.

If multiple regions are referenced, the user can navigate between them sequentially using a control, e.g., a built-in arrow control.

The visual indication (e.g., highlighting) persists during the user session and is re-rendered dynamically to maintain performance efficiency across concurrent users. Each user session is isolated such that navigation actions in one browser instance do not affect others.

III.c.3 Integration with Bounding Box Architecture

This interactive context linking and document navigation components operate in conjunction with the document-to-AI positional correlation and visual highlighting components, reusing the same coordinate data captured during OCR text extraction.

The pre-existing bounding box metadata—comprising page, paragraph, and X/Y coordinate data—serves as the foundation for both:
Passive visualization (bounding boxes displayed during initial review), and
Active navigation (real-time context linking when a user clicks a navigation icon).

By maintaining consistent coordinate mapping across both systems, the platform ensures high fidelity between AI reasoning, data provenance, and user-visible context.

### III.c.4 User Experience and Behavior

Real-time verification: users can click the navigation icon to see where the AI has sourced its answer, reducing uncertainty and validation time.

Sequential navigation: For answers supported by multiple regions, users can traverse between evidence areas using on-screen arrow controls.

Visual cues: Visual indications, e.g., bounding boxes as colored outlines, are drawn dynamically, visually distinguishing AI-referenced content from surrounding text.

Session persistence: The coordinates and highlights persist during the session but do not alter the underlying document

### III.c.5 Technical Architecture

To implement the visual navigation and coordinate mapping described above, the system may employ an exemplary technical stack comprising the following components:
ReactJS Client: acts as the frontend interface that executes the navigation logic, auto-scrolls to coordinate regions, and/or dynamically renders bounding boxes.
PostgreSQL Database: serves as the persistence layer that stores the AI-coordinate crosswalk, including region count, X/Y bounds, and sequence mapping for auditability and retrieval.
Azure Functions/API Layer: functions as the orchestration layer that supplies pre-fetched coordinate and metadata data to the client during page load.
AI Model Output (JSON): Provides the structured data payload containing the coordinate references and linkage information to map specific text regions to the model's generated responses.

The navigation process may be asynchronous and operate independently of server-side re-computation, ensuring low latency and high concurrency performance.

The system exhibits one or more technical effects and/or benefits including:
Bi-Directional Context Mapping—Creates an interactive link between AI-generated answers and their supporting document regions, enabling explainable and verifiable AI decision-making.
Multi-Region Evidence Navigation—Allows users to step through multiple contextual locations supporting a single AI output, reinforcing model transparency.
Preloaded Metadata for Instant Response—Leverages pre-cached coordinate data for immediate navigation without additional API latency.
Client-Side Rendering and Auto-Scrolling—Enables real-time highlighting, smooth page navigation, and seamless user experience across concurrent sessions.
Unified Provenance Framework—Integrates tightly with existing OCR-based bounding box data, ensuring consistent and traceable mapping between unstructured text and structured AI output.
Regulatory and Workflow Benefit—Enhances auditability and clinician confidence by visually grounding AI recommendations in the underlying documentation.

Example: An AI generated answer states a patient has a history of aggression. This feature may allow a user to click the icon associated with that answer to reveal that there are, for instance, 4 sections in the source document supporting the finding. The user can then actuate navigation controls (e.g., up/down arrows) to sequentially navigate through the document to those sections. In this visual arrangement, the answers and navigation controls (e.g., icons/arrows) may remain static on one side of the interface (e.g., the right, pane), while the document viewer on the other side (e.g., the left pane) adaptively navigates and highlights the relevant regions in real-time. See, e.g., FIGS. 13A and 13B.

### IV. Matching and Routing

The system (e.g., 150, 300B, 400, 3400) is configured to perform for automated candidate matching by correlating structured referral responses with organization-defined acceptance criteria. Specifically, the system (e.g., candidate matching engine 440) correlates structured answers—whether entered manually or extracted via AI—to capability questions with organization-defined capabilities (configurable to the facility level) as noted on a centralized database where each question has an acceptance capability noted per facility. The system evaluates each response in (substantially) real time against preconfigured thresholds and presents visual indicators reflecting alignment (e.g., by “green”), cautionary thresholds (e.g., by “yellow”), or incompatibility (e.g., “red”). This enables referral coordinators and clinical teams to rapidly determine patient-facility fit without manually comparing each data point against internal admission standards.

In some embodiments, the system supports multiple evaluation modes, such as a “Targeted Evaluation” mode and a “Network Search” (or “broadcast”) mode. In the “Targeted Evaluation” mode (common in direct referral workflows), the referral data package specifies a single target service provider. In this case, the system retrieves the capability specifications for that single target node and evaluates the referral solely against those constraints to generate a recommendation (e.g., Accept/Reject) for that specific provider. Conversely, in the “Network Search” mode, the system compares the referral against the capability specifications of a plurality of service providing nodes (potential providers) to identify one or more best-fit candidate nodes.

### IV.1 Data Configuration and Architecture

The system is built on a centralized data schema within a database environment, such as a PostgreSQL environment. Each organization maintains a set of acceptance criteria records, which may be applied globally across the organization or specifically overridden at the facility level. In some embodiments, each rule record includes one or more items including: a facility ID and/or organizational ID; a question reference ID (linked to a standardized questionnaire or AI-extracted data field); an accepted value or range definitions (e.g., Yes=Red, No=Green, >12=Green, 10-11=Yellow, <10=Red); a value type (Boolean, numeric, date, categorical); an operator type (equal to, greater than, less than, before, after); and an associated color outcome (Green, Yellow, Red). Rules may be configured directly through an administrative user interface (UI), enabling non-technical users to define or update acceptance criteria without modifying underlying logic. When rules are created at the organization level, they are automatically propagated to all associated facilities unless locally overridden.

### IV.2 Input Sources and Evaluation

The system evaluates responses from two primary input streams: user-entered data (structured questionnaire responses entered through the user interface) and AI-extracted data (fields generated through an AI-based (e.g., LLM-based) content extraction process). When a new response is entered or updated, the value is passed to an API-based rules evaluation service (e.g., executing within rules engine 480 utilizing the logic evaluation module 482). The service queries the acceptance criteria database for a corresponding rule tied to the specific facility and question ID. If a match exists, the rule evaluation service (or referred to as an engine, executing within rules engine 480) applies the defined logic to determine the response's compliance status and assigns it a visual indicator (e.g., a color value).

### IV.3 Range and Condition Handling

Rules can include discrete value matching (e.g., “Yes=Red”, “No=Green”); numeric thresholds (e.g., “<8=Green”, “8-9=Yellow”, “>9=Red”); and/or date-based conditions (e.g., “Before Sep. 1, 1998=Green”, “After Sep. 1, 1998=Red”). Each rule needs an explicit match based on simple logic (i.e., a direct, single-variable comparison), facilitating rapid, item-by-item evaluation. Compound logic conditions—complex scenarios that need simultaneous evaluation of multiple variables (e.g., using Boolean operators such as AND/OR)—are supported in the platform's broader rules engine (e.g., rules engine 480 shown in FIG. 4). By standardizing rule structure, the system ensures uniform interpretation across facilities and question types, regardless of the data source.

### IV.4 Visualization and User Interaction

When the evaluation is complete, results may be displayed inline with each corresponding question within the referral review UI. A response may be visually tagged with, e.g., a colored indicator. For example, “Green” indicates the response meets or exceeds facility acceptance criteria; “Yellow” indicates the response is within a cautionary or borderline range; and “Red” indicates the response does not meet facility criteria. Additionally or alternatively, a summary panel (e.g., positioned at the top of the interface) aggregates the results, displaying the total count of green, yellow, and red items for the current referral. The interface allows users to filter or sort responses by visual inspection (e.g., by color) to quickly focus on disqualifying or borderline criteria. This visualization layer is integrated with the platform's frontend (e.g., ReactJS), ensuring updates occur dynamically as new data is entered or AI results are parsed. The summary panel may include a summary of aggregated results for assessments done for multiple facilities ranked by compatibility or likelihood of readmission.

### IV.5 Data Persistence and Reporting

Evaluation results are stored in the database alongside the response data. Specifically, results including determined statuses (corresponding to visual indicators, e.g., color-coding) are persisted and linked to the original referral record, the specific facility/organization, the evaluated field, and the evaluation timestamp. These stored results can be used for:
Reporting and analytics (e.g., acceptance pattern analysis, bottleneck identification)
Quality assurance (e.g., monitoring frequency of red/yellow results)
AI performance validation (comparing automated extraction results against organizational thresholds).
This persistent data model supports both real-time display and historical review across referral activity.

### IV.6 Technical Workflow and System Configuration

The system executes a technical workflow to enable automated capability matching. During rule configuration, a user defines acceptance rules for a facility (or an organization) and question(s) through the administrative UI. During response capture, answers are generated through manual entry and/or AI extraction. The process then moves to API evaluation, where the rules evaluation service retrieves applicable criteria and computes results including acceptance status (e.g., corresponding to a visual indication, such as color-coding). In the UI display phase, results may be shown inline per question. Additionally or alternatively, aggregated counts may be displayed at, e.g., the top of the screen. Regarding database storage, part of or all evaluations and statuses are stored for audit and analytics. This workflow is fully contained within the platform's architecture (e.g., Azure), ensuring secure, (substantially) real-time processing without external dependencies.

FIG. 14 illustrates a flow diagram 1400 for capability matching via direct database correlations, according to some embodiments. The process initiates with two inputs: a referral question response 1410 (which may be user-entered or AI-extracted) and the acceptance criteria database 1420 (which houses the specific rules). These inputs are fed into the evaluate criteria at 1430, where the system logic correlates the specific response against the facility-specific rule. The flow 1400 proceeds to a decision block labeled “Determined?” at 1440. Based on the evaluation logic, the system determines a specific status which is categorized into one of three visual outputs: Green (indicating alignment) 1450, Yellow (indicating a cautionary status) 1460, or Red (indicating incompatibility) 1470.

The disclosed configuration offers several technical effects and/or advantages. It provides (substantially) real-time rule-based correlation by instantly evaluating structured or AI-extracted responses against facility-specific criteria through API-driven logic. The system enables facility-level flexibility with centralized governance, supporting organization-wide standardization while allowing granular facility overrides. A unified visual indication schema (e.g., color-coding schema) is utilized to deliver an intuitive visual interface that translates complex criteria into actionable decision signals. Furthermore, integrated multi-source evaluation seamlessly applies the same acceptance logic to both human-entered and AI-generated data fields. The system also possesses date and numeric threshold intelligence, supporting temporal and quantitative comparisons using parameterized logic. By maintaining a persistent analytical framework, the system stores evaluation and outcome, enabling advanced reporting and trend analysis. This results in regulatory and operational efficiency, reducing manual review time, standardizing intake evaluation, and providing defensible audit records for clinical and administrative teams.

The following examples illustrate the correlation logic. In a first example, a question asks, “Does the patient require a respirator?” with possible answers of yes/no. In the database, the organization has indicated for a specific facility that “Yes=Red” and “No=Green”; consequently, if the answer is “Yes,” the frontend reflects the red indicator.

In a second example, a question asks, “How many L/min oxygen does the patient require?” where the answer is a number field. On the acceptance database, the organization may have indicated for this facility that: <8 equals Green; 8-9 equals Yellow; and >9 equals Red. The UI reflects the corresponding color based on the numeric value extracted from the referral package.

### IV.7 Geospatial Matching and Proximity Logic

In some embodiments, the system (e.g., candidate matching engine 440 shown in FIG. 4) is configured to filter or rank potential care centers based on geospatial proximity. The system operates using a configured map or database of organizational locations, where each facility is associated with geospatial coordinates (e.g., latitude and longitude). Patient location data is derived from the normalized referral records (e.g., address fields extracted via the OCR/NLP pipeline) or integrated data feeds.

Upon initiation (e.g., when a new referral is ingested), the system queries the organizational location map and applies a proximity algorithm to determine the distance between the patient's location and each available care center. The computation may utilize a cloud-based geolocation service (e.g., an API-based mapping service).

Based on this assessment, the system is configured to identify, e.g.:
(a) The nearest care center to the patient;
(b) A ranked list of care centers within a specified distance threshold (e.g., within 10 miles); or
(c) A subset of centers filtered by both distance and configurable attributes (such as service type, bed availability, or payer participation).
(d) Evaluation of external service proximity. The system may evaluate capability based on the distance to required third-party services outside the facility. The system may evaluate capability based on the distance to required third-party services. For example, if a patient requires dialysis and the facility does not provide it on-site, the system calculates the driving distance to the nearest contracted dialysis center. If the distance exceeds a defined threshold (e.g., >10 miles), the compatibility indicator for “Dialysis Support” is marked as “Fail”. Merely by way of example, if a patient requires “Dialysis,” the system calculates the distance from the candidate facility to the nearest contracted dialysis center. If the distance exceeds a defined threshold (e.g., >10 miles), the system generates a “Fail” or “Red” compatibility indicator for that specific service requirement. See, e.g., geographic mapping as described elsewhere herein.

The results are returned to the application layer and may be displayed within the referral interface (e.g., as a map visualization or sorted list). Additionally or alternatively, the proximity data is used as a weighted parameter for automated system actions, such as assigning a referral to the nearest center or filtering recommendations based on capability matching.

Based on the determined compatibility indicators (e.g., 3 Red Flags), the system generates a recommendation message. In some embodiments, this recommendation is initially displayed internally to a clinical reviewer via the user interface (e.g., a “System Recommendation: Reject” banner). The interface provides interactive mechanisms (e.g., “Approve” or “Decline” buttons) allowing the user to validate the system's recommendation. Upon user confirmation (e.g., clicking “Decline”), the system generates the external transmission (e.g., the rejection message) to the source node.

Additionally or alternatively, based on the determined status (e.g., visually indicated by Green/Yellow/Red in a user interface), the system may generate a recommendation message. In some embodiments, this message is a disposition recommendation displayed internally to a reviewer user (e.g., “System Recommends: Decline”). The interface provides interactive mechanisms (e.g., “Approve” or “Decline” buttons) allowing the user to act on this recommendation. If the user selects “Decline” based on the system's finding, the system may then generate an external transmission to the source node.

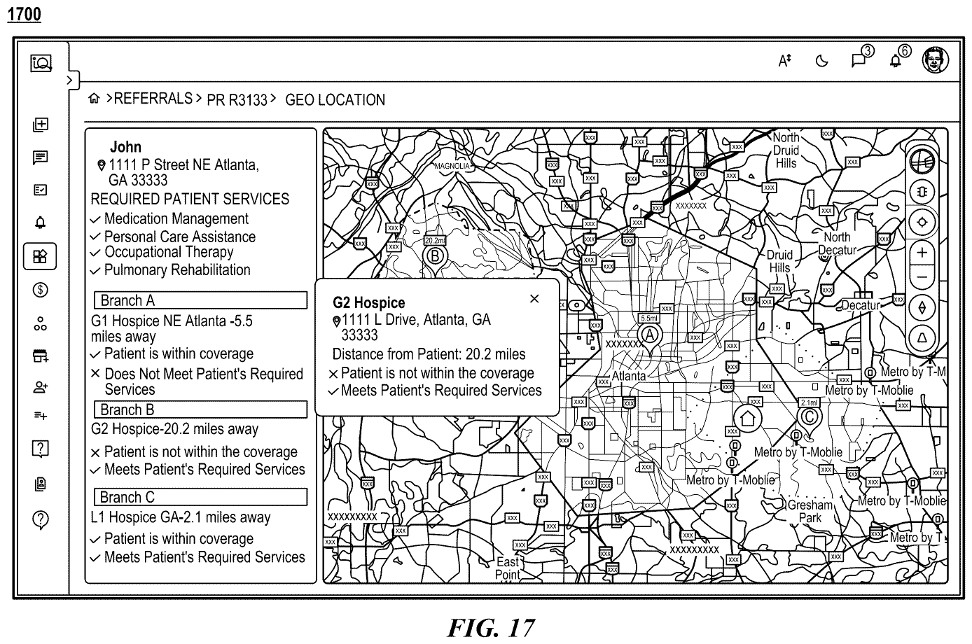

FIGS. 15-17 show exemplary user interfaces illustrating geospatial matching as part of candidate matching, according to some embodiments. FIG. 15 illustrates an exemplary patient referral profile interface 1500. The interface displays demographic and location data 1510 for a patient (e.g., address, city, state, zip code) derived from the referral packet. A user-selectable element, such as the “Check Coverage Area & Routing” button 1520, is provided to initiate the geospatial query described above, triggering the transition to the map views shown in FIGS. 16 and 17. FIG. 16 shows a map visualization 1600 displaying the patient's location relative to multiple facilities, with visual indicators (e.g., polygons or radius circles) denoting service areas. FIG. 17 illustrates a detailed view 1700 where specific facilities are ranked by distance (e.g., “5.5 miles away”), along with status indicators verifying if the patient falls within their specific coverage area (e.g., “Patient is within coverage”).

### V. AI-Driven Decision Support

In some embodiments, the system includes a decision support engine (e.g., decision support engine 450) configured to generate predictive analytics regarding one or more factors including, e.g., clinical risk, reimbursement forecasting, cost estimation, or the like, or a combination thereof. By leveraging the AI-driven data extraction and RAG-based validation frameworks described elsewhere in the present document, the system automates the calculation of standardized indices (e.g., LACE scores, PDPM classifications) and projects financial outcomes (e.g., medication costs) prior to patient admission.

### V.a AI-Driven LACE Scoring and Dynamic Clinical Risk Stratification System (Automated LACE Index)

In some embodiments, the system is configured to automatically estimate the risk of hospital readmission. For instance, the system is configured to automatically determine a LACE index-a standardized predictive model used to estimate the risk of hospital readmission. The LACE score (or LACE index) is a standardized predictive tool commonly used within healthcare to determine if a patient is likely to readmit back to a hospital within 30 days of discharge. “L” stands for length of stay, “A” stands for acuity, “C” stands for comorbidities, and “E” stands for emergency visits. It is used by skilled nursing facilities (SNFs) to evaluate the severity of a patient's condition and determine if they are ready for discharge. The system is configured to automate the LACE scoring with AI and gives users the ability to modify the score if they have additional context not included in the referral packet. The workflow may be implemented by the clinic risk module 452 of the decision support engine 450 as illustrated in FIG. 4.

In some embodiments, the system is configured to automatically calculate a LACE index through the integration of AI-driven data extraction, retrieval-augmented validation, and facility-level configuration within a healthcare referral management platform. The system dynamically computes the patient's readmission risk using data derived from referral documentation, ensuring accuracy, transparency, and interoperability across multiple care facilities. It enables automated decision support and workflow triggering based on calculated risk levels. This eliminates the need for manual data entry and ensures accurate, consistent scoring aligned with standardized clinical parameters.

### V.a.1 Data Ingestion and Normalization

The system accepts referral data from both structured and unstructured sources, including, e.g., HL7/FHIR data feeds, EMR/EHR feeds, EHR-integrated discharge summaries, PDF-based referral forms, or the like, or a combination thereof.

An AI-driven extraction pipeline (e.g., OCR and NLP pipeline) extracts relevant data elements—such as encounter duration, admission type, diagnoses, and prior emergency visits—and normalizes these into the platform's proprietary JSON schema. AI-powered NLP models interpret unstructured referral text with high accuracy to extract structured values corresponding to the LACE variables (Length of stay, Acuity, Comorbidities, and Emergency visits).

A normalized record includes metadata linking the extracted data to the originating document for auditability and visual verification (e.g., using the same bounding box and coordinate-mapping architecture described elsewhere in the present document). This unified schema allows the LACE scoring algorithm to process all inputs consistently, regardless of their original data format.

### V.a.2 Standardization and Validation via RAG Integration

The platform maintains a Retrieval-Augmented Generation (RAG) knowledge base containing validated scoring frameworks and reference tables derived from external clinical sources. The RAG layer (and the broader “RAG Integration” system described herein may correspond to the ML engine 430 (or referred to as RAG engine 430) shown in FIG. 4. For instance, the RAG model stores:
a. Standard LACE Index weights and scoring thresholds.
b. Validated definitions for each component (L, A, C, E).
c. Historical benchmarks and regional readmission rates for contextual calibration.

When the system initiates scoring, the computation engine references this knowledge base to ensure alignment with standardized, evidence-based criteria. Specifically, the knowledge base functions as a “Reference LACE Model,” acting as a dynamic reference layer that retrieves up-to-date scoring standards and allowing for contextual comparison—for example, adjusting risk thresholds based on SNF patient demographics or regional readmission rates.

In addition to the canonical LACE Index, the RAG layer may also support organization-specific rubrics for related scoring models (e.g., facility-defined risk indices or acuity-based prioritization).

### V.a.3 AI Scoring Process and Calculation

Once validated parameters are available, the system applies the LACE computation algorithm dynamically, referencing historical data to contextualize risk:
a. L (Length of Stay): Calculated from hospitalization duration within the referral or discharge record (e.g., discharge summaries or hospital encounter data).
b. A (Acuity of Admission): Determined from admission source and encounter classification (e.g., emergency vs. elective or presenting condition (the primary medical reason, symptom, or diagnosis that brought the patient to the hospital in the first place)).
c. C (Comorbidities): Derived from ICD-10 or SNOMED-coded problem lists using standardized mappings (e.g., Charlson or Elixhauser indices).
d. E (Emergency Visits): Counted from prior encounter data extracted from EHR feeds or historical referral text (e.g., visits in the past six months).

The resulting numeric total may produce a risk score (e.g., 0-19), and/or categorical risk tiers (Low, Moderate, High). The system may also generate a confidence score, reflecting data completeness and model certainty. In some embodiments, the system facilitates continuous learning, refining predictions over time based on real-world readmission outcomes.

The computations are executed within a cloud computing environment (e.g., Azure environment), leveraging containerized functions for orchestration and real-time response.

### V.a.4 User Interface and Explainability

The calculated LACE score is displayed within the referral management interface. A user can expand the scoring view to view a breakdown of the contributing variables, including, e.g.:
a. Extracted source values (e.g., “LOS: 5 days”)
b. Assigned component scores (e.g., “L=3, A=2, C=4, E=3”)
c. The total calculated score and corresponding risk category
d. Confidence percentage and version of scoring rubric used

By exposing both inputs and algorithmic logic, the interface promotes clinical transparency and auditability. SNF staff can use the risk score to determine admission readiness, allocate care resources, and plan post-admission monitoring.

### V.a.5 Workflow Integration and Rules Engine Automation

When the LACE score is finalized and/or validated by a user, it is written to the patient's referral record within a database (e.g., PostgreSQL database). If the referral is shared across multiple facilities within the same organization, the computed score automatically propagates, allowing all facilities to access the same result without redundant computation.

The system integrates with the platform's rules engine (e.g., rules engine 480), which enables organizations to configure automated triggers based on score thresholds. Examples include:
a. Automatically flagging “High Risk” patients for clinical review.
b. Sending alerts or notifications to transitional care teams, or triggering automated workflows—such as care coordination alerts or referrals to transitional care teams for high-risk patients.
c. Adjusting routing priority for referral acceptance workflows.

These automations are user-defined, configurable per organization, and executed securely within the cloud-based rules orchestration service.

### V.a.6 Validation and Storage

A computed LACE score, along with its associated inputs, thresholds, and data references, is stored in the platform's relational database under the patient's record. For instance, the stored data includes:
a. Individual component values (L, A, C, E)
b. Source document references and extraction metadata
c. Final score and category
d. User ID and timestamp for validation events
e. Applicable scoring rubric (standard or custom)

This structure supports historical tracking, outcome analysis, and longitudinal trend evaluation at both facility and organization levels.

### V.a.7 Technical Workflow

In a referral intake phase, the system receives structured or unstructured referral data. In a data extraction phase, the system performs the AI-driven extraction pipeline (e.g., OCR and NLP pipeline) processes to extract structured values into JSON format. In a RAG validation phase, the system validates parameters against the Reference LACE Model stored in the RAG knowledge base. The model maintains standard scoring tables, parameter weightings, and historical outcomes for benchmarking. In a computation phase, the system determines a LACE score using a standardized or custom algorithm. In a display phase, the system displays, or provides for display, results in the UI (with or without a breakdown of components). In a user validation phase, a user confirms and finalizes the LACE score. In a storage and propagation phase, the LACE score is written to database. The score may be shared across facilities in the same organization or different organizations. In an automation phase, the rules engine triggers predefined workflows based on the results and relevant thresholds.

The disclosed configuration offers several technical effects and/or advantages including, e.g.:
End-to-End Automation: Eliminates manual LACE scoring by automatically extracting and computing all needed parameters directly from referral documentation. For instance, an NLP model is used to interpret unstructured referral text with high accuracy.
RAG-Backed Validation: Ensures, by the RAG model, LACE calculations adhere to the latest validated scoring tables while allowing contextual adjustment through dynamic retrieval.
Organizational Customization: Supports custom scoring rubrics per organization or facility while maintaining centralized schema and interoperability.
Real-Time Explainability: Displays a breakdown of the scoring logic and data sources to enhance clinician trust and regulatory compliance. This includes traceability through spatial links between responses and source information.
Rules-Driven Workflow Integration: Transforms static risk scores into actionable clinical triggers via the platform's integrated rules engine.
Cross-Facility Synchronization: Propagates validated scores across all facilities within an organization, preventing duplicate effort and ensuring consistent data visibility.
Scalable Risk Stratification: Supports high-volume referral processing without manual scoring.
Continuous Learning: System refines predictions over time based on real-world readmission outcomes.
Cloud-Native Scalability: Leverages the platform's cloud-native environment for rapid, secure, and scalable computation at enterprise scale.

FIG. 18 is a flow diagram illustrating an exemplary LACE score system workflow 1800, according to some embodiments. FIG. 18 contains components similar to those in FIG. 5, and like components are labeled with like reference numerals, the description of which is not repeated for brevity.

After the system reads the OCR document text at 535, the process 1800 moves to 1835, where the system fetches LACE-specific questions and AI prompts from the database and converts them into a standardized format (e.g., JSON). Concurrently, or in coordination, the system retrieves custom indexed data from the RAG storage (544), which may include organization-specific LACE parameters (1841, 1842, 1843) to contextually validate or adjust the scoring logic.

At 1840, a payload is generated combining the document text, the Question JSON, and general prompts. The payload may also include the custom indexed data including organization-specific LACE parameters. This payload is transmitted to the AI model (e.g., Azure Open AI GPT 4.1 mini model) at 545. Upon successful processing (handling retries at 550 if needed), the system receives the LACE score as a JSON response at 1855. The system converts this response at 560 and persists the AI-generated answers to the database (565). This workflow 1800 includes displaying the calculated LACE Score to end users at 1875 and the queue message is marked as processed and deleted at 570.

FIGS. 19 and 20 show exemplary user interfaces 1900 and 2000 including LACE scores, according to some embodiments.

### V.b AI-Driven PDPM Calculator and Automated Reimbursement Forecasting System (PDPM Calculator)

In some embodiments, the system is configured to automate the projected Patient-Driven Payment Model (PDPM) (reimbursement) calculations, according to some embodiments. Specifically, the system is configured to automate the PDPM classification and reimbursement forecasting process for Skilled Nursing Facilities (SNFs) for patient pre-admission. By leveraging artificial intelligence, the system extracts, interprets, and maps clinical and functional information from referral documentation into standardized PDPM inputs, computes projected reimbursement rates (e.g., Medicare Part A reimbursement rates), and presents users with transparent, evidence-backed financial forecasts. This eliminates manual entry of diagnostic and functional data while maintaining compliance with CMS-defined PDPM standards and enabling explainable, audit-ready reimbursement predictions. The system configuration and workflow for AI-driven PDPM estimation may be similar to the LACE score determination. The workflow may be implemented by the financial forecasting module 454 of the decision support engine 450 as illustrated in FIG. 4.

### V.b.1 Data Intake and Normalization

The PDPM calculator integrates directly with the platform's data pipeline (e.g., an existing Azure-based pipeline), leveraging optical character recognition (OCR), natural language processing (NLP), and large language models (LLMs) to ingest both structured and unstructured referral data. Examples of acceptable sources include: (a) EHR-integrated feeds (HL7, FHIR); (b) Hospital discharge summaries; (c) Physician and therapy notes; and (d) Manually uploaded PDFs or faxed referral documents.

Extracted data is normalized into the platform's proprietary schema (e.g., JSON schema), preserving traceability to the source document and maintaining consistent data representation across input types. Each data element retains its positional metadata (page, line, or coordinate) to enable intra-document navigation and source validation during user review.

### V.b.2 PDPM Classification Logic

In some embodiments, the system implements CMS-defined PDPM rules and Minimum Data Set (MDS) mappings as its baseline logic model. The AI engine may perform several parallel tasks including, e.g.:
Clinical Category Assignment: Identifies the patient's primary diagnosis and relevant comorbidities to assign the PDPM clinical category (e.g., Orthopedic Surgery, Neurologic, Medical Management).
Functional Status Scoring: Parses Section GG (Functional Abilities and Goals) of the MDS assessment to extract mobility and self-care metrics from discharge or therapy notes, inferring missing values where contextually supported.
Discipline Mapping: Allocates the patient's expected care needs across, e.g., Physical Therapy (PT), Occupational Therapy (OT), Speech-Language Pathology (SLP), Nursing, and Non-Therapy Ancillary (NTA) categories.
Case-Mix Group and CMI Calculation: Applies PDPM algorithms to compute the Case Mix Index (CMI) for each discipline and determine the corresponding reimbursement group codes.
Projected Cash Flow Forecasting: Using the calculated CMIs, the system projects day-by-day reimbursement rates and computes both average per diem (PPD) and total length-of-stay revenue for each referral case.

### V.b.3 AI-Driven Inference and Human Validation

The system uses AI inference to estimate PDPM parameters when information is not explicitly stated within the referral document (e.g., suggesting functional score ranges or therapy discipline probabilities). An AI-derived value is flagged for human validation before inclusion in the final PDPM computation.

The validation interface displays both: (a) The proposed value and confidence level; and (b) The source citation (highlighted text or bounding box location) within the document using the platform's intra-document navigation system. This ensures transparency and clinical accuracy while maintaining compliance with CMS documentation requirements.

V.b.4 Integration with RAG Reference Model

The system's PDPM logic is dynamically supported by a Retrieval-Augmented Generation (RAG) model hosted in the cloud environment (e.g., Azure). The RAG layer maintains: (a) Official CMS PDPM rules and mappings (regularly updated via API or file synchronization); (b) Wage index data and regional reimbursement tables; and (c) Internal scoring models that extend beyond PDPM for organization-specific forecasting metrics.

During the AI scoring process, the RAG model retrieves the current version of applicable rule sets and fee schedules, ensuring that calculations reflect up-to-date regulatory and geographic adjustments. Additionally or alternatively, the system can provide AI-assisted recommendations (non-binding, compliance-safe guidance)—for example, alerting users to verify certain therapy or coding details that may place the patient near a reimbursement threshold.

### V.b.5 Output Generation and Visualization

The PDPM calculator produces an interactive reimbursement forecasting dashboard, accessible within the referral management interface. The dashboard includes: (a) Detailed component breakdowns for each PDPM category (L, GG, Clinical Category, Comorbidity Group, NTA); (b) Calculated CMIs and reimbursement group codes; (c) Visualizations of per-diem rates, total projected reimbursement, and average daily PPD; and (d) Day-by-day projected revenue curves across the anticipated stay duration.

A calculation element is accompanied by traceable evidence, allowing users to navigate directly to the corresponding document section that informed the input (via bounding boxes and tooltips).

### V.b.6 Workflow Integration and Automation

Once validated, the PDPM score and projected reimbursement outputs are stored in the database (e.g., PostgreSQL) as part of the patient's referral record. These outputs can:
(a) Trigger automated actions in the platform's Rules Engine, such as alerts to clinical or financial teams for high- or low-margin cases.
(b) Export structured projection data (e.g., JSON or CSV format) for downstream use in billing, financial modeling, or data warehousing systems.
(c) Be shared automatically with other facilities within the same organization that received the same referral, eliminating redundant processing.

The computations and automation may be partially or fully executed within the cloud environment (e.g., Azure), ensuring compliance, data isolation, and scalability.

### V.b.7 Explainability and Compliance

A PDPM calculation may be fully explainable and audit-ready. Users can perform one or more including:
(a) Expand any PDPM component to view its calculated value, formula, and reference data;
(b) Navigate to the specific document segment supporting that input;
(c) Review AI-inferred fields before final confirmation.

This explainable framework ensures compliance with CMS documentation standards, supports audit defense, and fosters clinician trust in AI-assisted financial analysis.

### V.b.8 Technical Workflow

The workflow proceeds as follows:
Referral Data Ingestion: The OCR/NLP pipeline extracts clinical data from structured and unstructured inputs.
Normalization: Extracted content is converted into standardized JSON format.
RAG Reference Retrieval: Latest CMS PDPM rules and wage data are fetched from the RAG knowledge base.
AI Classification and Inference: The model assigns clinical category, discipline mapping, and CMI.
Human Validation: A user verifies or corrects AI inferences and inputs.
Computation and Visualization: The PDPM algorithm is executed; output is displayed in a real-time dashboard.
Storage and Automation: Results are stored per patient and routed to the Rules Engine for workflow triggers or export.

The disclosed configuration offers several technical effects and/or advantages including, e.g.:
Automated Regulatory Intelligence: Applies CMS-defined PDPM rules dynamically through AI and RAG integration, ensuring real-time compliance and consistency across facilities.
AI-Powered Data Extraction and Inference: Automatically identifies diagnoses, comorbidities, and Section GG values from unstructured text and infers likely PDPM inputs when incomplete.
Explainable Financial Forecasting: Displays every calculation component with linked source evidence and supporting documentation, allowing for transparent clinical and financial review.
Actionable Integration: Enables real-time workflow automation (via Rules Engine) based on PDPM results, driving faster financial decision-making and care planning.
Centralized and Multi-Facility Synchronization: Stores validated PDPM outputs within shared organizational data structures, providing consistent forecasting visibility across facilities.
Scalable, Cloud-Native Architecture: Executes all processes securely within the cloud environment (e.g., Azure), utilizing event-driven orchestration for efficient, high-volume referral processing.
Intelligent, Compliance-Safe Recommendations: Offers AI-generated insights on near-threshold cases to guide user review without exceeding regulatory or ethical boundaries.

FIG. 21 is a flow diagram illustrating an exemplary AI-powered PDPM calculation workflow 2100, according to some embodiments.

At 2110, the system ingests the referral document (e.g., via EHR feeds, discharge summaries, or uploaded PDFs). At 2120, the system normalizes the extracted data into a standardized format (e.g., the platform's proprietary JSON schema), preserving positional metadata for traceability.

At 2130, the system retrieves the latest PDPM rules, mappings, and wage index data from the Retrieval-Augmented Generation (RAG) knowledge base. This ensures that the subsequent logic utilizes the most current CMS-defined standards.

At 2140, the system performs inference to estimate PDPM parameters that may not be explicitly stated in the text (e.g., inferring Section GG functional scores from therapy notes).

As illustrated in FIG. 21, the logic may split into parallel processing streams to handle the multi-faceted PDPM algorithms. For instance, the system applies AI classification logic at 2150 (e.g., assigning the clinical category or mapping disciplines) which informs the computation of the reimbursement projection at 2160. Simultaneously or sequentially, the system may execute additional computation cycles at 2180 based on the inferred data.

The streams converge at 2170 for human validation. At this stage, the user reviews the AI-generated classifications, inferred values, and financial projections—aided by the transparent source links—to confirm or adjust the data before finalization.

FIG. 22 shows an exemplary user interface 2200 for a PDPM calculator, according to some embodiments. FIG. 23 is a user interface 2300 showing exemplary PDPM rates by day, according to some embodiments.

### V.c Medication Pullouts

In some embodiments, the system includes a “Medication Pullouts” feature—an AI-powered medication need and cost estimation system. This feature leverages the AI-driven extraction pipeline (including, e.g., OCR, NLP, and/or generative AI capabilities as described elsewhere herein) to pull medication content from the referral packet and presents a medication list with pricing data (e.g., average wholesale price or acquisition cost) to give a comprehensive cost analysis of the patient's medications. The workflow may be implemented by the cost estimate module 456 of the decision support engine 450 as illustrated in FIG. 4.

In some embodiments, the system (e.g., via the decision support engine) leverages cloud-based Artificial Intelligence (AI) models (e.g., generative AI models, large language models, neural networks, or other machine learning architectures) to analyze patient referrals at SNFs and automatically determine if a patient may need ongoing medications after admission. Using, e.g., advanced natural language processing (NLP) and/or clinical inference models provided by the cloud-based AI models, the system identifies prescribed or recommended medications from unstructured referral data and calculates both individual and/or aggregate drug cost estimates.

An exemplary workflow is as follows.

### V.c.1 Referral Data Processing

The system ingests patient referral data from various sources, including, e.g., EHR feeds, PDFs, discharge summaries, or physician notes. The cloud-based AI models (e.g., generative AI models, large language models, neural networks, or other machine learning architectures) perform entity extraction to identify medications, dosages, and frequency patterns from the unstructured text.

The system matches the extracted medications against standardized drug dictionaries stored in an internal RAG model (e.g., the RAG knowledge base as described elsewhere in the present document).

### V.c.2 Medication Need Determination

The system (e.g. decision support engine 450) evaluates patient conditions, diagnoses, and treatment plans to confirm which medications are needed for ongoing care. Additionally or alternatively, the system flags potential missing prescriptions based on diagnosis correlations (e.g., identifying a diagnosis of diabetes and flagging a potential missing prescription for insulin or metformin).

### V.c.3 Cost Calculation Using Pricing Data

The system (e.g., RAG knowledge base) maintains a continuously updated repository of pricing data, such as National Average Drug Acquisition Cost (NADAC) data, which may be refreshed on a periodic basis (e.g., monthly) or initiated by a user or triggered automatically by an external API (e.g., when updates are available). When a medication is identified, its unit cost and average daily dosage are retrieved from an internal RAG model (e.g., the RAG knowledge base described elsewhere herein).

The system then computes at least one of the following including: (a) the estimated daily cost for each medication; (b) the weekly total cost (e.g., a 7-day projection); (c) any medication exceeding a defined cost threshold is automatically highlighted as a high-cost drug, or the like, or a combination thereof.

### V.c.4 Output & Reporting

Results are displayed in the SNF referral management dashboard with a structured breakdown including, e.g.:
(a) Medication name;
(b) Dosage and frequency;
(c) Daily and weekly estimated cost; and
(d) Visually indicated (e.g., highlighted) high-cost items.

Reports can be exported or integrated into billing and pharmacy systems for pre-admission financial planning.

The disclosed configuration offers several technical capabilities including, e.g.:
Advanced NLP Models: Used for accurate medical text interpretation and drug mapping from referrals.
RAG Integration: Stores and retrieves standardized medication pricing and reference rules from datasets such as NADAC.
Dynamic Cost Computation: Calculates individualized and aggregated drug costs per patient in real time.
High-Cost Drug Detection: AI-driven cost flagging assists SNFs in proactive budget management and formulary control.

FIG. 24 is a flow diagram illustrating an exemplary medication cost RAG system workflow 2400, according to some embodiments. FIG. 24 contains components similar to those in FIG. 5, and like components are labeled with like reference numerals, the description of which is not repeated for brevity.

After the system reads the OCR document text at 535, the process 2400 proceeds to 2435, where the system fetches medication-specific questions and AI prompts from the database and converts them into a standardized format (e.g., JSON). Concurrently, or in coordination, the system references the RAG storage (544), which utilizes a pricing dataset, such as the NADAC list (e.g., a CSV updated monthly) (2442), to validate costs or retrieve pricing standards.

At 2440, a payload is generated combining the document text, the Question JSON, and general prompts. The payload can also include the pricing dataset. This payload is transmitted to the AI model (e.g., Azure Open AI GPT 4.1 mini model) at 545. Upon successful processing (handling retries at 550 if needed), the system receives a drug list and cost data as a JSON response at 2455. The system converts this response at 560 and persists the AI-generated answers to the database at 565.

The workflow 2400 includes displaying the drug list with price to end users at 2475. As illustrated, this display operation may incorporate organization-specific high-cost medication thresholds (2481, 2482, 2483) to automatically flag or highlight medications exceeding defined cost limits before the queue message is marked as processed and deleted at 570.

FIGS. 25 and 26 show exemplary user interfaces 2500 and 2600 including medication information, according to some embodiments.

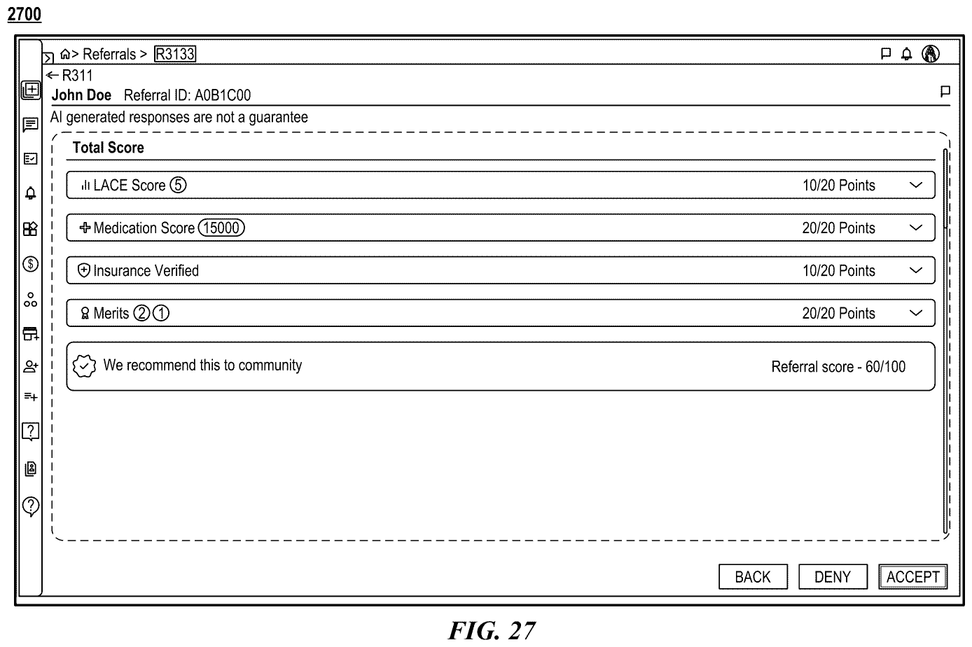

FIG. 27 shows an exemplary user interface 2700 illustrating a comprehensive referral scorecard, according to some embodiments. The interface aggregates the outputs from the various AI-driven scoring modules described herein into a unified view for decision-makers.

As shown, the scorecard displays a Total Score (e.g., “Referral score—60/100”) which may be a composite metric derived from multiple weighted factors. Illustrated components include the LACE Score (e.g., “10/20 Points”), which reflects the readmission risk calculated via the workflow of FIG. 15, “Insurance Verified” (e.g., “10/20 Points”), which indicates a result of a PDPM assessment, and the Medication Score (e.g., “20/20 Points”), which may correspond to the cost analysis or complexity derived from the workflow of FIG. 21.

The interface may further show an AI-generated recommendation (e.g., “We recommend this to community”) along with actionable decision buttons (e.g., “BACK,” “DENY,” “ACCEPT”) to facilitate immediate workflow routing based on the aggregated intelligence.

### V.d AI-Driven Medical Equipment Matching and Automated Acquisition

In some embodiments, the system (e.g., medical equipment management module 458 of decision support engine 450) further includes a) configured to analyze patient referrals, determine if the patient requires durable medical equipment (DME) for ongoing care, and facilitate the automated sourcing or purchasing of such equipment. This feature automates the connection between clinical need identification and supply chain fulfillment (e.g., purchase endpoints), reducing delays in care and administrative burden.

### V.d.1 AI-Driven Need Analysis

When a new patient referral is received (e.g., at an SNF), the system automatically ingests and parses referral documentation including, e.g., clinical notes, discharge summaries, and physician orders. The referral data is standardized and mapped into structured fields (e.g., diagnosis, mobility status, wound care needs, respiratory requirements) for downstream analysis. To achieve this, the system utilizes an AI engine (e.g., the generative AI models described elsewhere herein) to evaluate the patient's clinical documentation—ingested and normalized via, e.g., the OCR/NLP pipeline—to determine the likelihood of needing specific medical equipment. The model analyzes factors including, e.g.:
(a) Diagnosis-specific indicators (e.g., identifying a diagnosis of COPD implies a need for an oxygen concentrator; a post-op hip fracture implies a walker);
(b) Functional status assessments (e.g., mobility scores, Activities of Daily Living (ADLs), and fall risk);
(c) Clinical interventions (e.g., usage of wound vacs, infusion pumps, or respiratory therapy); and
(d) Past utilization patterns derived from similar patient populations.

The system produces a recommendation with a confidence score, visually indicating (e.g., by highlighting) required or optional equipment to support patient safety and recovery.

### V.d.2 Acquisition and Vendor Integration

In addition to identification of medical equipment needed, the system is configured to send structured data to “purchase endpoints” or vendor APIs. This allows the system to automatically connect care teams to potential vendors, present specific product options (e.g., via a “Medical Equipment RAG System” that cross-references equipment lists/catalogs), and facilitate the purchase transaction directly within the platform workflow.

FIG. 28 illustrates a flow diagram illustrating an exemplary AI-powered medical equipment (ME) RAG system workflow, according to some embodiments. FIG. 28 contains components similar to those in FIG. 5, and like components are labeled with like reference numerals, the description of which is not repeated for brevity.

After the system reads the OCR document text at 535, the process 2800 proceeds to 2835, where the system fetches medical equipment-specific questions and AI prompts from the database and converts them into a standardized format (e.g., JSON). Concurrently, or in coordination, the system references the RAG storage (544), which utilizes a medical equipment list or catalog (e.g., a CSV updated periodically (e.g., daily/weekly), or initiated by a user or triggered automatically by an external API) (2842) to validate equipment availability or specifications.

At 2840, a payload is generated combining the document text, the Question JSON (ME requirement questions), and prompts. The payload may include the medical equipment list or catalog information. This payload is transmitted to the AI model (e.g., Azure Open AI GPT 4.1 mini model) at 545. Upon successful processing (handling retries at 550 if needed), the system receives a Medical Equipment (ME) list as a JSON response at 2855. The system converts this response at 560 and persists the AI-generated results to the database at 565.

The workflow 2800 includes displaying the suggested ME for the facility (e.g., SNF) at 2875. As illustrated, this display operation may facilitate downstream acquisition steps, such as presenting a Medical Equipment vendor list at 2885 and enabling the purchase of the equipment from a vendor at 2880 before the queue message is marked as processed and deleted at 570.

The workflow 2800 may include performing dynamic capability checks. For example, if the system identifies a need for “Negative Pressure Wound Therapy,” the system can trigger a real-time API call to a medical equipment vendor to verify inventory availability. If the equipment is available for order, and delivery by a required date, the compatibility indicator is marked as “Pass” (or “Conditional Pass”); if unavailable, it is marked as “Fail”.

### VI. Configurable Rules Engine and Automated Workflows

In some embodiments, the system includes a configurable rules engine (e.g., rules engine 480 as illustrated in FIG. 4) configured to enable conditional automation of referral-related processes within the application environment. This engine allows authorized users to define one or more rules, each comprising a set of trigger condition(s) and one or more resulting action events. For instance, the system includes a configurable, event-driven rules engine that enables organizations to define and execute automated workflows based on, e.g., referral data attributes, system events, time-based conditions, geographical conditions, or the like, or a combination thereof. Examples of system events include state-based transition (e.g., the workflow status of a record, such as a referral moving from “New” to “Processing” or “Admitted”), data field modifications (e.g., updates to specific data attributes, such as changes in insurance payer information, patient demographics, clinical indicators), temporal events (e.g., time-based conditions which function as internal system events, triggering when specific intervals elapse (e.g., “Last update>3 days ago”) or when a target time is reached relative to a scheduled event (e.g., 48 hours before discharge)).

This engine allows non-technical users to build sophisticated conditional automations through a visual interface, while maintaining the ability to execute asynchronously across multiple communication and interoperability channels. The rules engine serves as an automation layer of the platform, enabling user-defined workflows without requiring new software development. It supports both simple single-trigger workflows and complex multi-condition logic structures, integrating seamlessly with the platform's communication, task management, and external API layers.

### VI.a Rule Configuration and Data Architecture

A rule is stored as a structured object in the platform's database (e.g., PostgreSQL), represented as a JSON-based data structure. This structure defines various parameters including, e.g.: (i) a unique rule ID; (ii) organizational and facility scope; (iii) trigger definition(s); (iv) action definition(s); (v) execution mode (e.g., asynchronous or conditional chain); and (vi) audit metadata (e.g., creator, timestamps, and last execution logs).

Rules are interpreted dynamically as potential trigger events occur throughout the application. When an event matches one or more defined conditions, the rule is queued for evaluation. Once verified, the system executes the corresponding action(s) asynchronously within a background worker or serverless function (e.g., an Azure Function), ensuring that primary application performance is not disrupted.

### VI.b Trigger Definition and Evaluation Logic

Rules may contain one or more trigger conditions which define the circumstances under which an action may or need to execute. Trigger conditions are evaluated in real time whenever related data changes or scheduled evaluation intervals occur. The rules engine may continuously monitor application events and state transitions to identify trigger matches. A potential trigger event is parsed through a logical evaluation module (e.g., logical evaluation module 482 executing within rules engine 480, which determines whether all Boolean conditions are satisfied before invoking any action.

To process these triggers, the rules engine may utilize the logical evaluation module to determine whether all applicable Boolean conditions are satisfied before invoking any action. The logical evaluation module may be configured to assess various criteria including, e.g.:
State-Based Triggers: Activated by field or status changes (e.g., “Referral Status=Processing”).
Time-Based Triggers: Activated when specified time intervals elapse (e.g., “Last update>3 days ago,” “Trigger 48 hours before discharge”).
Data-Driven Triggers: Based on defined field values (e.g., “Insurance=Medicaid,” “Patient Age>65,” “Hospital Name=ABC Medical Center”).
Composite Logic: Users can combine multiple conditions using Boolean and algebraic logic structures, such as (A AND B) OR (C AND F), enabling complex workflow definitions.

In some embodiments, the rules engine operates in coordination with the decision support engine 450. For example, the decision support engine 450 may generate probabilistic outputs (e.g., a “High Risk” score from Clinic Risk Module 452 or a cost projection from Cost Estimate Module 456). The logical evaluation module 482 of the rules engine 480 can ingest these outputs as data-driven triggers.

Example 1: If clinic risk module 452 output>80% (condition), then rules engine 480 triggers an “Urgent Review” Task (Action).

Example 2: If candidate matching engine 440 identifies a facility within 5 miles (geographic condition) AND decision support engine 450 confirms capacity (capacity condition), THEN rules engine 480 automates the referral assignment.

### VI.c Action Definition and Orchestration Layer

A rule may define one or more resulting actions which execute when the trigger conditions are met. In some embodiments, actions are modular and executed through a multi-modal outbound orchestration layer, allowing the system to perform communication, data manipulation, or task generation across multiple channels and systems. Examples of supported action types include, among others, e.g.:
Intra-Application Notifications: Displays real-time messages to designated users or teams within the application.
External Communications: Sends SMS messages or emails via dedicated API connectors.
Task Creation: Automatically generates task records associated with the referral record, including metadata such as due date, assigned user, and priority.
API Push Actions: Sends structured data payloads to third-party systems or EHRs using secure API connectors.
Record Updates: Automatically updates related entities, such as community assignments or referral statuses.
Data Enrichment and Re-Analysis: Triggers a task for a user or an automated agent to gather missing information (e.g., requesting a missing lab result). Upon receipt of the new data, the rules engine can trigger a re-execution of the AI analysis pipeline to update the analysis record and compatibility indicators.
Resource Procurement: Initiates an external workflow to order required resources. For example, if the AI identifies a need for a specific “Wound Vac” that is not currently in inventory but is required for acceptance, the system can trigger an API call to a medical equipment vendor to check availability or place an order, thereby resolving the capability gap.

An action type may have a standardized payload structure. In some embodiments, users can define which contextual fields (e.g., patient name, referral ID, referral status, payer, assigned user) are included in the outgoing message or data packet. An exemplary message template which a user may use to configure a notification action is as follows: “The referral status has updated to <status>. Please continue processing for patient <patient name>.” Actions can execute in parallel or sequentially, depending on the rule configuration, and are handled through modular connectors that manage the needed security, payload formatting, and error handling for each channel.

### VI.d Visual Configuration Interface and Auditing

The rules engine provides a graphical user interface (GUI) that enables users to define rules without programming knowledge. Exemplary UI features include:
a) a Visual Logic Builder: Allowing users to define Boolean expressions using drag-and-drop condition blocks or dropdown selectors (e.g., “If Referral Status=Admitted AND Payer=Medicare”).
b) Action Templates: Allowing users to select from predefined action types (e.g., “Send Notification,” “Create Task,” “Push to API”) and configure variable fields.
c) Logic Validation Mode: Allows users to test logical consistency and review example evaluations (e.g., “If Referral Status=Admitted AND Payer=Medicaid→Send SMS”), Action Templates, and a Logic Validation Mode.

Once saved, rules are compiled into structured JSON objects and activated for continuous monitoring. The interface may be configured to support logic validation testing to confirm syntax and field references prior to activation, by full sandbox simulation or otherwise.

### VI.e Execution and Asynchronous Processing

The system executes actions based on a two-step qualification process: detecting a trigger event and evaluating specific logic conditions. Upon detection of a qualifying trigger event, the rules evaluation service—configured as an API-based service operating within the platform's cloud environment (e.g., Azure)—may utilize the logical evaluation module to evaluate the applicable trigger conditions. If these conditions are satisfied, the service generates (e.g., via contextual variable injection or task record creation) and executes the defined actions asynchronously. This ensures (substantially) real-time responsiveness while preventing workflow delays in other system operations. Execution operations may generally include, e.g.:
(a) Detection of a qualifying trigger event;
(b) Logical evaluation of all Boolean and algebraic conditions;
(c) Queuing of validated actions into the background job processor;
(d) Execution of each action via its assigned communication module (SMS, email, API, etc.); and
(e) Confirmation of successful delivery or logging of errors for retry or audit.

An execution event is recorded in an audit log with rule ID, timestamp, action type, success/failure status, and user visibility permissions.

### VI.f Auditing, Security, and Compliance

Rule definitions, modifications, and executions are logged for compliance and traceability. Examples of audit data include:
(a) Rule creator and modification history;
(b) Execution timestamps and output logs;
(c) Communication payload records (excluding sensitive PHI content where applicable); and
(d) Action success/failure events.

Access to rule creation and modification is restricted through role-based access control (RBAC), ensuring that only authorized administrative users can alter automation logic. For example:
System Administrators: May be granted full permissions to create, edit, activate, and delete global automation rules affecting all facilities.
Facility Managers: May be restricted to viewing existing rules or configuring specific parameters (e.g., adjusting notification thresholds) for their assigned facility only, without the ability to alter the underlying Boolean logic.
Standard Users (e.g., Intake Coordinators): May have “read-only” access or no visibility into the rules engine configuration, preventing accidental modification of critical workflows while still being subject to the automated actions (e.g., receiving notifications).

This hierarchical security model ensures that while the automation impacts the entire organization, the control over its logic remains centralized and secure.

### VI.g Extensibility

The rules engine and outbound orchestration layer are designed for modular extensibility, supporting future trigger and action types that may be subsequently configured. The architecture accommodates new communication channels or interoperability methods, such as:
(a) Secure Direct Messaging or HL7/FHIR endpoints;
(b) Chat-based communication (e.g., Teams or Slack integrations); and
(c) Advanced interoperability frameworks (e.g., real-time EHR updates).

This configuration provides long-term adaptability as healthcare integration standards and technologies evolve.

### VI.e Technical Workflow

An exemplary workflow is as follows:

Rule Creation: Authorized users define conditional logic and actions via visual GUI. This interface enables non-technical users to build sophisticated conditional automations (e.g., using drag-and-drop condition blocks) without requiring new software development. Users can configure Boolean expressions, select action templates, and validate logic directly within the portal.

Storage: rules are stored as structured JSON data in a database (e.g., PostgreSQL). A rule object can include parameters including a unique rule ID, organizational scope, trigger definitions, and action definitions. This structured storage allows for dynamic retrieval and interpretation by the system at runtime.

Monitoring: The rules engine (e.g., the rules evaluation service) monitors referral data attributes, system events, and time-based conditions. This monitoring may occur in (substantially) real-time. The monitoring may observe state transitions (e.g., status changes), the elapsed time since specific events to identify potential trigger matches, when a target time is reached relative to a scheduled event, or the like, or a combination thereof.

Trigger Evaluation: Upon detection of a qualifying event (trigger event), the system performs a trigger evaluation where Boolean logic is evaluated. The logical evaluation module parses the event data against the defined conditions (e.g., (A AND B) OR C) to definitively determine if the criteria for execution are met. This may include evaluating data-driven triggers such as capacity or geographic location.

Asynchronous Execution: Matching rules queued and executed in the background. If the conditions are satisfied, matching rules are queued and executed in the background. The system utilizes a background job processor (e.g., within an Azure environment) to handle these tasks asynchronously. This architecture ensures real-time responsiveness for the user while preventing the automation logic from disrupting the performance of the primary application.

Action Delivery: The system utilizes modular connectors to dispatch notifications, tasks, or API updates. An outbound orchestration layer manages the specific protocols for each channel-sending SMS or emails via external gateways, pushing payloads to third-party APIs, or creating internal task records. This operation may also include contextual variable injection, where dynamic data (e.g., patient names) is inserted into the action payload.

Audit Logging: All actions and outcomes are recorded for compliance review. The system generates a comprehensive audit trail including execution timestamps, rule IDs, action types, success/failure statuses, or the like, or a combination thereof. This data is maintained to support regulatory compliance and security oversight.

The disclosed configuration offers several technical effects and/or advantages including, e.g.:
Dynamic No-Code Automation—Enables non-technical users to create complex, algebraically structured workflow automations without software development.
Multi-Modal Orchestration Layer—Manages secure, asynchronous message delivery and API communication across multiple channels and systems.
Real-Time Event Responsiveness—Executes trigger evaluations continuously, enabling true event-driven automation in referral workflows.
Modular Extensibility—Future-proofs the system for emerging communication standards and interoperability frameworks.
Time and State-Based Triggering—Supports both event-driven and time-elapsed triggers, enabling highly flexible automation patterns.
Contextual Variable Injection—Allows users to include live patient, referral, or payer data directly within outbound messages or API payloads.
Transparent and Auditable—Provides complete audit trails of rule creation, modification, and execution for regulatory compliance and security oversight.
Scalable Cloud Architecture—Executes all rules asynchronously within the Azure environment, ensuring scalable, fault-tolerant automation for high-volume healthcare workflows.

FIG. 29 is a flow diagram illustrating an exemplary basic rules workflow 2900, according to some embodiments. The workflow 2900 represents the lifecycle of a rule from definition through execution and logging.

The process 2900 begins with rules definitions at 2910. Here, an authorized user defines conditional logic and actions via the visual GUI, and the rules are stored as structured JSON data (e.g., in PostgreSQL).

At 2920, the process 2900 includes performing event monitoring. The rules engine continuously monitors referral data attributes, system events, and time-based conditions to identify potential triggers. This operation feeds into the decision block at step 2930.

At 2930, the process 2900 includes determining if the condition evaluation is met. The system parses the event through the logical evaluation module to check if the Boolean or algebraic conditions defined in step 2910 are satisfied.

If the condition is not met (No), the process 2900 returns to event monitoring at 2920 to await further changes or events.

If the condition is met (Yes), the process 2900 proceeds to step 2940.

At 2940, the process 2900 includes executing an action. Matching rules are queued and executed asynchronously (e.g., via background job processors). Modular connectors dispatch notifications, tasks, API updates, or record modifications. As illustrated by the arrow returning to 2920, the process 2900 includes continuing to monitor for new events even as actions are executed, ensuring (substantially) real-time responsiveness.

At 2950, the process 2900 includes performing logging. All actions, outcomes, success/failure events, and communication payloads are recorded in the audit log for compliance review and security oversight.

FIGS. 30-32 show exemplary user interfaces for rules input and configuration, according to some embodiments.

FIG. 30 illustrates a rules management dashboard 3000. This interface provides a centralized view of existing automation rules across an organization. As shown, the dashboard displays a tabular list including the “Rule Name,” a text-based “Description” of the logic (e.g., “Triggers when status is set to . . . ”), the “Community” (facility) scope, and the resulting “Action” type (e.g., Task, Email, Note).

In addition to the rules table, the interface serves as a configuration hub, displaying a navigation menu (e.g., a top tab bar) that provides access to various system administration modules. As illustrated, these modules include “Question Bank,” “Word Bank,” “High RiskAdmission List,” “App Configuration,” and “Rules Engine” view. This layout allows authorized administrators to toggle between defining automation rules and managing other critical system definitions (such as risk lists or question templates) within a unified environment. The interface further includes controls to search for specific rules, filter rules by community, create new rules (via the “+NEW RULE” button), and manage the lifecycle of existing rules (e.g., edit, delete, or pause/resume execution).

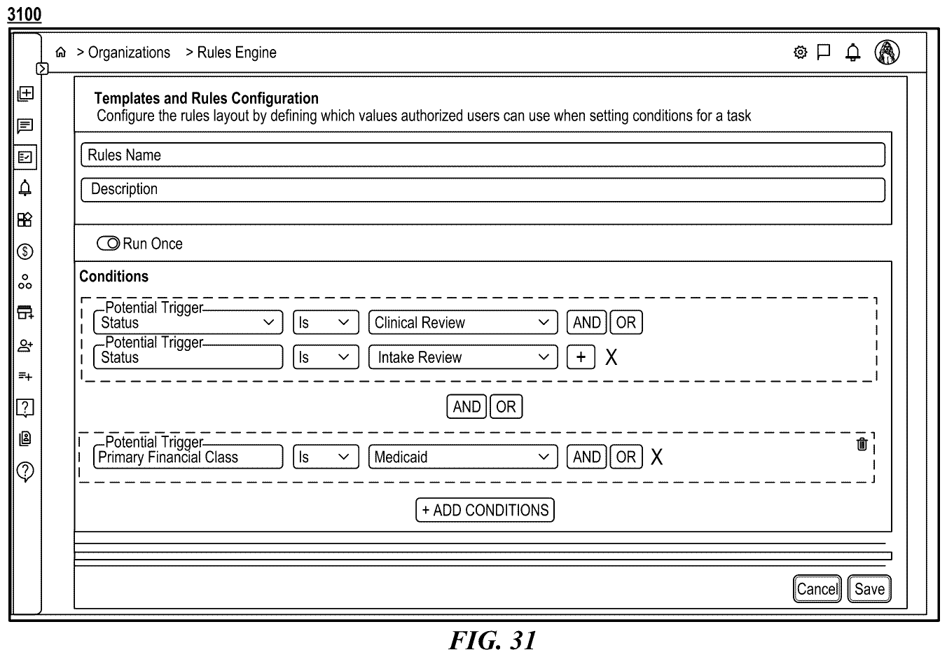

FIG. 31 illustrates a rule configuration interface 3100 featuring a visual logic builder. This interface corresponds to the trigger definition phase, enabling non-technical users to construct complex Boolean logic without coding. The interface includes fields for Rule Name and Description, followed by a “Conditions” section. As depicted, users can select potential triggers (e.g., “Status,” “Primary Financial Class”) and define values using dropdowns. The interface illustrates the creation of composite logic using “AND” and “OR” operators to group conditions. For example, the figure depicts a logic structure evaluating: (Status Is ‘Clinical Review’ OR Status Is ‘Intake Review’) AND (Primary Financial Class Is ‘Medicaid’).

FIG. 32 illustrates an action definition interface 3200, specifically depicting the configuration of a “Create Task” action. This interface allows users to define the parameters of the automated event that executes when the trigger conditions (defined in FIG. 31) are met. As shown, the user can configure specific task metadata, including the Task Name, Description, and Task Type. Furthermore, the interface enables the configuration of assignment logic (“Assign To”), priority levels, and dynamic due dates (e.g., defined as an offset, such as “00 Days” from the time of task creation), thereby integrating the automation into the workflow.

In some embodiments, the rules engine includes a population monitoring system designed to identify specific patients or cohorts (e.g., “high risk” or “frequent flyer” populations) upon intake and trigger specialized workflows. This system integrates the rules engine's event-monitoring capabilities with a customer-defined “unique population” database.

FIG. 33 illustrates a flow diagram of a population monitoring workflow 3300, according to some embodiments. The workflow 3300 demonstrates how the system correlates incoming referrals against a pre-defined list of individuals to automate alerts and custom processes.

The process 3300 begins at 3310, where a new referral is received in the system. This event corresponds to the ingestion phase described herein. The system maintains a Customer “unique population” database within the system at 3320. This database allows organizations to define specific lists of patients (e.g., a “High Risk Admission List” or a “Do Not Admit” list) based on identifiers such as Name, Date of Birth (DOB), and Social Security Number (SSN).

At 3330, the system performs a “Correlation match?” decision. It compares the identifiers from the incoming referral at 3310 against the records in the unique population database at 3320.

If no match is found (No), the process 3300 proceeds to 3340, where the system allows the referral to continue standard workflow (e.g., proceeding to clinical review as described in Section III).

If a match is found (Yes), the process moves to 3350. Here, the system activates a Visual indicator on record and Customer defined processes. For example, a “High Risk” icon or banner (as shown in FIG. 44) may be displayed on the referral card to alert users immediately.

The workflow 3300 then evaluates a secondary condition at 3360: Did patient admit?(or is the patient being admitted?). If the patient is not admitted (No), the standard workflow continues at 3340. However, if the patient is admitted (Yes), the system triggers Customer defined processes (rules engine) at 3370.

Operation 3370 represents the execution of automated actions defined within the rules engine (as described in Section VI). These actions may include:

SMS message with variable content: Sending a text alert to a care coordinator (e.g., “High risk patient [Name] admitted”).

Email with variable content: Notifying leadership or risk management teams.

Intra-app notification with variable content: Displaying a dashboard alert to the admissions team.

API with variable content: Pushing a status update to an external EMR or care management system.

### VII. ADR Management

Insurance/payor organizations commonly deny claims and send a request back to the provider asking for more documentation to support the claim. This documentation is most frequently found within a referral packet itself for the first billable claim, but often providers must seek additional documentation elsewhere. This process is poorly regulated and providers miss revenue due to poor quality or timeliness of the process.

The system (e.g., 150, 300B, 400, 3400) may include a cloud-based, automated workflow module (e.g., ADR management engine 470 in FIG. 4) for managing additional documentation requests (ADRs) generated by insurance payors during the billing cycle. The module enables users to create, track, fulfill, and analyze ADRs through a structured interface that integrates tightly with existing referral and clinical documentation systems. This module facilitates a systematic and auditable process for satisfying insurer documentation requests, reducing administrative workload and ensuring regulatory compliance.

### VII.a ADR Event Creation and Initialization

ADR management begins when a user manually creates a “New ADR” event within the application. The ADR management engine 470 provides immediate access to the patient referral data and associated documentation repositories previously ingested. Upon event creation, the user specifies relevant attributes including, e.g., patient and community identifiers, payor information, pertinent dates, request ID, assigned staff/departments, claim IDs, and deadlines. This data initializes an ADR tracking record in the system's relational database (e.g., Database 460), establishing a unique identifier and associating the ADR with its originating referral and claim context.

### VII.b Automated Document Retrieval and Categorization

Once an ADR event is created, the engine 470 automatically evaluates the platform's document repository to locate relevant files associated with the patient or claim. A user-defined category schema determines which document types should be included-such as clinical notes, prior authorizations, lab results, or therapy logs. The user can manually modify categories or upload additional documentation as needed; each category entry is linked to metadata including file type, upload timestamp, and source record ID.

To ensure searchability, the engine 470 utilizes an integrated OCR engine (e.g., Apryse, hosted within the same Azure environment) to extract and render document content for review and verification. In this context, the OCR process may focus solely on text extraction and indexing rather than semantic interpretation, preserving both compliance and operational simplicity. All documents and extracted text are stored securely in object storage (e.g., Azure Blob Storage), indexed by ADR ID, and cross-referenced within the platform's relational data model.

### VII.c Packet Assembly and Submission

After document gathering, the engine 470 compiles all items into a payer-compliant packet. Documents are merged and ordered according to the insurer's specified submission format, defined within a configurable rules engine. Each section of the output packet may include an auto-generated cover page identifying the request type, patient, and claim number. The engine 470 supports various submission workflows, including, e.g.: (1) Direct digital upload to payer portals (via user action or future automation); (2) Secure file packaging for email or fax submission; and (3) Export to archive for manual submission and audit purposes. All packet generation activities may be logged and versioned for traceability.

### VII.d Workflow Management and Alerts

The ADR management engine 470 features an integrated workflow interface that allows users to track open requests, monitor progress, and assign responsibilities. Each ADR record contains a configurable task list. The system's rules engine 480 operates in coordination with the ADR management engine 470 to monitor deadlines and trigger automated notifications through multiple channels-including email, SMS, or in-application alerts. These events may be generated using platform-level automation functions and adhere to organization-defined escalation rules.

### VII.e Completion and Analytics

Upon completion of an ADR submission, the system records outcome details, storing all data in the relational database for subsequent analysis. The engine 470 provides dashboards and visual analytics to track metrics such as ADR timing, outcomes, and payer behavior, enabling continuous operational improvement.

### VII.f Technical Architecture

The ADR management engine 470 may operate within the same cloud-based, multi-tenant environment (e.g., Azure) as the primary platform. The engine 470 may include components including, e.g.: (1) App services (e.g., Azure App Services) for front-end interaction; (2) object storage (e.g., Azure Blob Storage) for document management; (3) relational databases (e.g., Azure SQL) for metadata; and (4) serverless functions (e.g., Azure Functions) for event-driven automation, OCR invocation, and packet assembly. All data transmissions use HTTPS/TLS encryption and token-based authentication. Tenant isolation is maintained through unique storage and database partitions for each customer, ensuring data segregation and HIPAA compliance.

### VIII. Clinical Pathway Review & Recommendation

In some embodiments, the system (e.g., 150, 300B, 400, 3400) is configured to operate as a clinical agent that identifies and ranks candidate care or clinical pathways by searching clinical history across multiple care providers. In this context, the terminology of the present disclosure may be adapted as follows:

The candidate node (or service providing node) represents a clinical pathway (e.g., a specific sequence of treatments, interventions, or care decisions) rather than a physical facility.

The node capability specification represents clinical pathway compatibility criteria (e.g., clinical protocols, inclusion/exclusion criteria for a treatment, or guidelines).

The compatibility indicator represents a clinical finding derived from the patient's data, such as prior clinical history, current conditions, or specific biomarkers.

The recommendation represents a recommended clinical pathway.

In operation, the system analyzes the electronic data package to extract relevant text chunks, which may include patient-related clinical events spanning multiple encounters across various service providing nodes. When generating the structured response or analysis record, synthesizing the relevant text chunks includes identifying at least one current medical condition based on temporal proximity to a present encounter (e.g., distinguishing between a historical “History of Pneumonia” and an active “Current Pneumonia” diagnosis).

The system determines whether the identified clinical findings (compatibility indicators) satisfy the clinical pathway compatibility criteria. If multiple clinical pathways are determined to be compatible, the system ranks the clinical pathways based on probabilistic factors, including historical frequency, outcome correlation (e.g., efficacy rates of the pathway for similar patient profiles), and patient trajectories associated with similar compatibility indicators. The verification interface visually associates the clinical findings (e.g., the specific prior clinical history) with the relevant text chunks on the document image, allowing the user to validate the evidence supporting the recommended clinical pathway.

### IX. Survey Preparedness and Documentation Standardization

In some embodiments, the system (e.g., 150, 300B, 400, 3400) is configured to support a survey preparation workflow to maintain inspection readiness. In this context, the terminology and functionality are adapted as follows:

The compatibility indicator represents a compliance status indicator, a deficiency risk indicator, or a documentation completeness indicator.

The node capability specification represents a predefined set of regulatory, accreditation, or policy-based deficiency criteria (e.g., CMS Conditions of Participation or Joint Commission standards).

The system utilizes these criteria for detecting and flagging missing, inconsistent, or non-compliant data elements in the documentation (identifying deficiency risks or documentation gaps).

The recommendation message comprises at least one remediation artifact selected from a template, checklist, policy excerpt, or standardized form associated with the detected documentation gap.

In an exemplary survey preparedness use case, the verification interface is configured to visually distinguish any compliance status or deficiency risk indicator that has a confidence score below a threshold or corresponds to a detected documentation gap. Furthermore, the system is configured to periodically receive updated data packages and rerun the analysis to monitor for newly introduced or unresolved deficiency risks, ensuring ongoing compliance.

In some embodiments, the system supports a documentation standardization workflow to maintain a clear, reviewable record throughout a full care episode. The system structures queries to gather relevant text from the data package (source documentation) and synthesizes the relevant text to complete documentation in a standardized format. Similar to the survey agent, the system compares indicators to the capability specification (e.g., documentation standards) to flag missing, inconsistent, or non-compliant data elements. The verification interface links back to the relevant text in the source to provide evidence. Crucially, in this embodiment, the validation input received from the user includes linking to additional supporting evidence (e.g., allowing a user to manually attach a specific lab result or progress note to verify a specific data element), thereby ensuring the standardized record is fully substantiated.

The disclosed configuration offers several technical effects and/or advantages including, e.g.:
(1) Integrated Document Discovery: Automatically links ADR events with existing referral documentation, eliminating redundant searches;
(2) Configurable Category Framework: Allows organizations to define and modify required document types dynamically, with manual overrides when needed;
(3) Automated Packet Assembly: Merges, sequences, and formats documentation according to payer-specific rules, adding automated cover pages for each section;
(4) Rule-Driven Alerts and Escalations: Employs a configurable rules engine to trigger notifications for deadlines and incomplete submissions across multiple communication channels;
(5) Cloud-Native OCR Integration: Extracts text content for indexing and validation using a hosted OCR service within the same secure environment;
(6) Comprehensive Workflow Tracking: Centralized interface for task assignment, progress tracking, and outcome recording ensures full lifecycle visibility; and
(7) Multi-Tenant Deployment: Leverages isolated data stores per customer with integrated security and compliance features aligned to HIPAA standards.

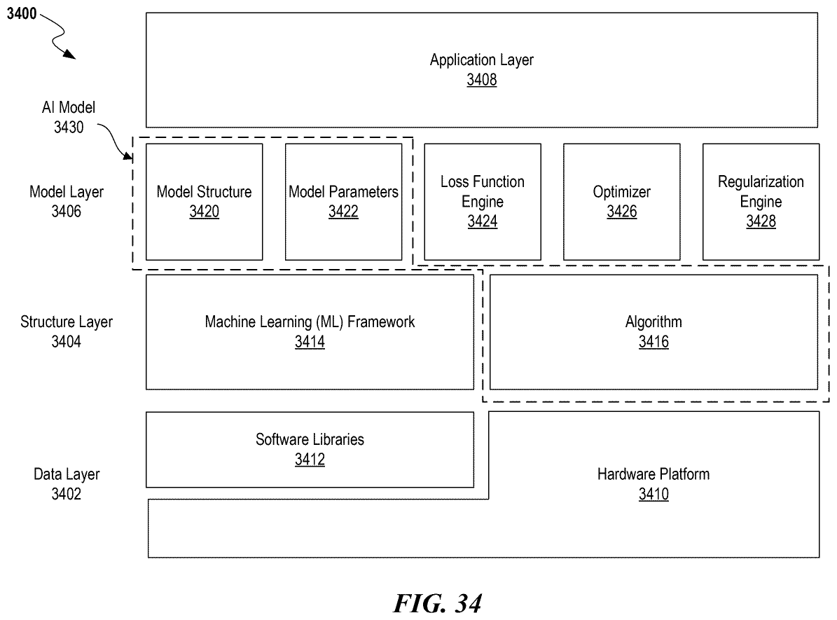

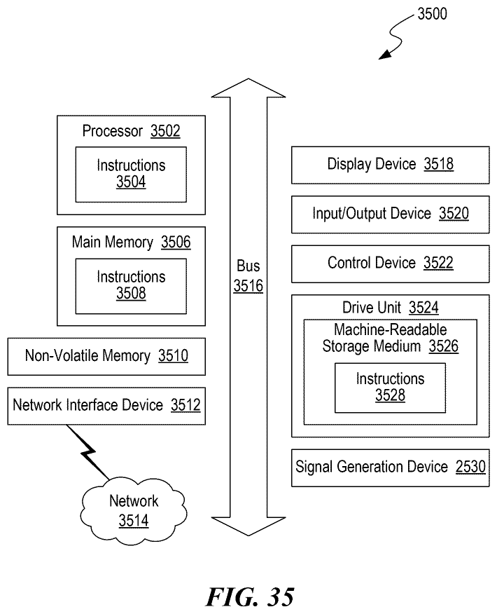

FIG. 34 is a block diagram that illustrates an example AI system that can implement aspects of the present technology. The AI system 3400 is implemented using components of the example computer system 3500 illustrated and described in more detail with reference to FIG. 35 (formerly FIG. 23). For example, the AI system 3400 can be implemented on the processor 3502 using instructions 3508 programmed in the memory 3506 illustrated and described in more detail with reference to FIG. 35. Likewise, implementations of the AI system 3400 can include different and/or additional components or be connected in different ways.

FIG. 34 illustrates a layered architecture of AI system 3400 that can implement AI models within the system 150 or 300B (e.g., the machine learning component as described in 220 of FIG. 2A/2B, referral microservice 344 of FIG. 3B), in accordance with some implementations of the present technology. The AI system 3400 may implement the functional engines illustrated in FIG. 4.

As shown, the AI system 3400 can include a set of layers, which conceptually organize elements within an example network topology for the AI system's architecture to implement a particular AI model 3430. Generally, an AI model 3430 is a computer-executable program implemented by the AI system 3400 that analyses data to make predictions. For example, the Retrieval-Augmented Generation (RAG) Engine 430 of FIG. 4 (including the vector embedding module 432 and analysis generation module 438) may be implemented as an AI model 3430. Information can pass through each layer of the AI system 3400 to generate outputs for the AI model 3430. The layers can include a data layer 3402, a structure layer 3404, a model layer 3406, and an application layer 3408.

The algorithm 3416 of the structure layer 3404 and the model structure 3420 and model parameters 3422 of the model layer 3406 together form an example AI model 3430. For instance, predictive scoring modules shown in FIG. 4, such as the LACE scoring module 452 or the PDPM module 454, may utilize specific algorithms 3416 (e.g., regression analyses or decision trees) to execute their respective risk and reimbursement calculations. The optimizer 3426, loss function engine 3424, and regularization engine 3428 work to refine and optimize the AI model 3430, and the data layer 3402 provides resources and support for application of the AI model 3430 by the application layer 3408.

The data layer 3402 acts as the foundation of the AI system 3400 by preparing data for the AI model 3430. In the context of the system shown in FIG. 4, the data layer 3402 may manage the storage and retrieval of the vector store index 434 and the node capability specifications stored in the storage device 442. As shown, the data layer 3402 can include two sub-layers: a hardware platform 3410 and one or more software libraries 3412. The hardware platform 3410 can be designed to perform operations for the AI model 3430 and include computing resources for storage, memory, logic and networking, such as the resources described in relation to FIG. 35. The hardware platform 3410 can process amounts of data using one or more servers. The servers can perform backend operations such as matrix calculations, parallel calculations (e.g., for the pairwise correlation determinations of the retrieval module 436 in FIG. 4), machine learning (ML) training, and the like. Examples of servers used by the hardware platform 3410 include central processing units (CPUs) and graphics processing units (GPUs). CPUs are electronic circuitry designed to execute instructions for computer programs, such as arithmetic, logic, controlling, and input/output (I/O) operations, and can be implemented on integrated circuit (IC) microprocessors. GPUs are electric circuits that were originally designed for graphics manipulation and output but may be used for AI applications due to their vast computing and memory resources. GPUs use a parallel structure that generally makes their processing more efficient than that of CPUs. In some instances, the hardware platform 3410 can include computing resources, (e.g., servers, memory, etc.) offered by a cloud services provider. The hardware platform 3410 can also include computer memory for storing data about the AI model 3430, application of the AI model 3430, and training data for the AI model 3430. The computer memory can be a form of random-access memory (RAM), such as dynamic RAM, static RAM, and non-volatile RAM.

The software libraries 3412 can be thought of suites of data and programming code, including executables, used to control the computing resources of the hardware platform 3410. The programming code can include low-level primitives (e.g., fundamental language elements) that form the foundation of one or more low-level programming languages, such that servers of the hardware platform 3410 can use the low-level primitives to carry out specific operations. The low-level programming languages do not require much, if any, abstraction from a computing resource's instruction set architecture, allowing them to run quickly with a small memory footprint. Examples of software libraries 3412 that can be included in the AI system 3400 include INTEL Math Kernel Library, NVIDIA cuDNN, EIGEN, and OpenBLAS.

The structure layer 3404 can include an ML framework 3414 and an algorithm 3416. The ML framework 3414 can be thought of as an interface, library, or tool that allows users to build and deploy the AI model 3430. The ML framework 3414 can include an open-source library, an application programming interface (API), a gradient-boosting library, an ensemble method, and/or a deep learning toolkit that work with the layers of the AI system facilitate development of the AI model 3430. For example, the ML framework 3414 can distribute processes for application or training of the AI model 3430 across multiple resources in the hardware platform 3410. The ML framework 3414 can also include a set of pre-built components that have the functionality to implement and train the AI model 3430 and allow users to use pre-built functions and classes to construct and train the AI model 3430. Thus, the ML framework 3414 can be used to facilitate data engineering, development, hyperparameter tuning, testing, and training for the AI model 3430. Examples of ML frameworks 3414 that can be used in the AI system 3400 include TENSORFLOW, PYTORCH, SCIKIT-LEARN, KERAS, LightGBM, RANDOM FOREST, and AMAZON WEB SERVICES.

Merely by way of example, the ML framework 3414 may include at least one ML-based language model. It may be noted that, while the term “language model” has been commonly used to refer to an ML-based language model, there could exist non-ML language models. In the present disclosure, the term “language model” can refer to an ML-based language model (e.g., a language model that is implemented using a neural network or other ML architecture), unless stated otherwise. For example, unless stated otherwise, the “language model” encompasses Large Language Models (LLMs).

A language model can use a neural network (e.g., a Deep Neural Network or DNN) to perform Natural Language Processing (NLP) tasks. A language model can be trained to model how words relate to each other in a textual sequence, based on probabilities. A language model may contain hundreds of thousands of learned parameters or, in the case of an LLM, can contain millions or billions of learned parameters or more. As non-limiting examples, a language model can generate text, translate text, summarize text, answer questions, write code (e.g., Python, JavaScript, or other programming languages), classify text (e.g., to identify spam emails), create content for various purposes (e.g., social media content, factual content, or marketing content), or create personalized content for a particular individual or group of individuals.

A type of neural network architecture, referred to as a “transformer,” can be used for language models. For example, the Bidirectional Encoder Representations from Transformers (BERT) model, the Transformer-XL model, and the Generative Pre-trained Transformer (GPT) models are types of transformers. A transformer is a type of neural network architecture that uses self-attention mechanisms to generate predicted output based on input data that has some sequential meaning (i.e., the order of the input data is meaningful, which is the case for most text input). The self-attention mechanism allows the model to weigh the importance of different words in a sequence relative to one another, regardless of their positional distance, thereby capturing long-range dependencies and contextual nuance within the referral data.

To process textual data, the language model utilizes tokenization and embeddings. Tokenization involves decomposing raw text (e.g., referral notes) into smaller units called tokens (e.g., words, sub-words, or characters). These tokens are then mapped to embeddings-dense, high-dimensional vectors of real numbers. Unlike traditional one-hot encoding, these embeddings capture semantic meaning, where tokens with similar meanings are positioned closer together in the vector space. This vector representation allows the model to perform mathematical operations on language, facilitating the “pairwise correlation” and “vector store index” operations described herein.

Although transformer-based language models are described herein, it should be understood that the present disclosure may be applicable to any ML-based language model, including language models based on other neural network architectures such as RNN-based language models.

The algorithm 3416 can be an organized set of computer-executable operations used to generate output data from a set of input data and can be described using pseudocode. The algorithm 3416 can include complex code that allows the computing resources to learn from new input data (e.g., temperature measurements and valve positions) and create new/modified outputs based on what was learned. In some implementations, the algorithm 3416 can build the AI model 3430 through being trained while running computing resources of the hardware platform 3410. This training allows the algorithm 3416 to make predictions or decisions without being explicitly programmed to do so. Once trained, the algorithm 3416 can run at the computing resources as part of the AI model 3430 to make predictions or decisions, improve computing resource performance, or perform tasks. The algorithm 3416 can be trained using supervised learning, unsupervised learning, semi-supervised learning, and/or reinforcement learning.

Using supervised learning, the algorithm 3416 can be trained to learn patterns (e.g., map input data to output data) based on labeled training data. The training data may be labeled by an external user or operator. For instance, a user may collect a set of training data, such as by capturing data from sensors, images from a camera, outputs from a model, and the like. In an example implementation, training data can include native-format data collected (e.g., in the form of temperature measurements or valve positions) from various source computing systems described in relation to FIG. 1. Furthermore, training data can include pre-processed data generated by various sensors of the system 100 described in relation to FIG. 1. The user may label the training data based on one or more classes and trains the AI model 3430 by inputting the training data to the algorithm 3416. The algorithm determines how to label the new data based on the labeled training data. The user can facilitate collection, labeling, and/or input via the ML framework 3414. In some instances, the user may convert the training data to a set of feature vectors for input to the algorithm 3416. Once trained, the user can test the algorithm 3416 on new data to determine if the algorithm 3416 is predicting accurate labels for the new data. For example, the user can use cross-validation methods to test the accuracy of the algorithm 3416 and retrain the algorithm 3416 on new training data if the results of the cross-validation are below an accuracy threshold.

Supervised learning can involve classification and/or regression. Classification techniques involve teaching the algorithm 3416 to identify a category of new observations based on training data and are used when input data for the algorithm 3416 is discrete. Said differently, when learning through classification techniques, the algorithm 3416 receives training data labeled with categories (e.g., classes) and determines how features observed in the training data (e.g., valve positions) relate to the categories. Once trained, the algorithm 3416 can categorize new data by analyzing the new data for features that map to the categories. Examples of classification techniques include boosting, decision tree learning, genetic programming, learning vector quantization, k-nearest neighbor (k-NN) algorithm, and statistical classification.

Regression techniques involve estimating relationships between independent and dependent variables and are used when input data to the algorithm 3416 is continuous. Regression techniques can be used to train the algorithm 3416 to predict or forecast relationships between variables. To train the algorithm 3416 using regression techniques, a user can select a regression method for estimating the parameters of the model. The user collects and labels training data that is input to the algorithm 3416 such that the algorithm 3416 is trained to understand the relationship between data features and the dependent variable(s). Once trained, the algorithm 3416 can predict missing historic data or future outcomes based on input data. Examples of regression methods include linear regression, multiple linear regression, logistic regression, regression tree analysis, least squares method, and gradient descent. In an example implementation, regression techniques can be used, for example, to estimate and fill-in missing data for machine-learning based pre-processing operations.

Under unsupervised learning, the algorithm 3416 learns patterns from unlabeled training data. In particular, the algorithm 3416 is trained to learn hidden patterns and insights of input data, which can be used for data exploration or for generating new data. Here, the algorithm 3416 does not have a predefined output, unlike the labels output when the algorithm 3416 is trained using supervised learning. Said another way, unsupervised learning is used to train the algorithm 3416 to find an underlying structure of a set of data, group the data according to similarities, and represent that set of data in a compressed format.

A few techniques can be used in supervised learning: clustering, anomaly detection, and techniques for learning latent variable models. Clustering techniques involve grouping data into different clusters that include similar data, such that other clusters contain dissimilar data. For example, during clustering, data with possible similarities remain in a group that has less or no similarities to another group. Examples of clustering techniques density-based methods, hierarchical based methods, partitioning methods, and grid-based methods. In one example, the algorithm 3416 may be trained to be a k-means clustering algorithm, which partitions n observations in k clusters such that each observation belongs to the cluster with the nearest mean serving as a prototype of the cluster. Anomaly detection techniques are used to detect previously unseen rare objects or events represented in data without prior knowledge of these objects or events. Anomalies can include data that occur rarely in a set, a deviation from other observations, outliers that are inconsistent with the rest of the data, patterns that do not conform to well-defined normal behavior, and the like. When using anomaly detection techniques, the algorithm 3416 may be trained to be an Isolation Forest, local outlier factor (LOF) algorithm, or K-nearest neighbor (k-NN) algorithm. Latent variable techniques involve relating observable variables to a set of latent variables. These techniques assume that the observable variables are the result of training on the latent variables and that the observable variables have nothing in common after controlling for the latent variables. Examples of latent variable techniques that may be used by the algorithm 3416 include factor analysis, item response theory, latent profile analysis, and latent class analysis.

The model layer 3406 implements the AI model 3430 using data from the data layer and the algorithm 3416 and ML framework 3414 from the structure layer 3404, thus enabling decision-making capabilities of the AI system 3400. The model layer 3406 includes a model structure 3420, model parameters 3422, a loss function engine 3424, an optimizer 3426, and a regularization engine 3428.

The model structure 3420 describes the architecture of the AI model 3430 of the AI system 3400. The model structure 3420 defines the complexity of the pattern/relationship that the AI model 3430 expresses. Examples of structures that can be used as the model structure 3420 include decision trees, support vector machines, regression analyses, Bayesian networks, Gaussian processes, genetic algorithms, and artificial neural networks (or, simply, neural networks). The model structure 3420 can include a number of structure layers, a number of nodes (or neurons) at each structure layer, and activation functions of each node. Each node's activation function defines how a node converts data received to data output. The structure layers may include an input layer of nodes that receive input data, an output layer of nodes that produce output data. The model structure 3420 may include one or more hidden layers of nodes between the input and output layers. The model structure 3420 can be an Artificial Neural Network (or, simply, neural network) that connects the nodes in the structured layers such that the nodes are interconnected. Examples of neural networks include Feedforward Neural Networks, convolutional neural networks (CNNs), Recurrent Neural Networks (RNNs), Autoencoder, and Generative Adversarial Networks (GANs).

The model parameters 3422 represent the relationships learned during training and can be used to make predictions and decisions based on input data. The model parameters 3422 can weight and bias the nodes and connections of the model structure 3420. For instance, when the model structure 3420 is a neural network, the model parameters 3422 can weight and bias the nodes in each layer of the neural networks, such that the weights determine the strength of the nodes and the biases determine the thresholds for the activation functions of each node. The model parameters 3422, in conjunction with the activation functions of the nodes, determine how input data is transformed into desired outputs. The model parameters 3422 can be determined and/or altered during training of the algorithm 3416.

The loss function engine 3424 can determine a loss function, which is a metric used to evaluate the AI model's performance during training. For instance, the loss function engine 3424 can measure the difference between a predicted output of the AI model 3430 and the actual output of the AI model 3430 and is used to guide optimization of the AI model 3430 during training to minimize the loss function. The loss function may be presented via the ML framework 3414, such that a user can determine whether to retrain or otherwise alter the algorithm 3416 if the loss function is over a threshold. In some instances, the algorithm 3416 can be retrained automatically if the loss function is greater than the threshold. Examples of loss functions include a binary-cross entropy function, hinge loss function, regression loss function (e.g., mean square error, or quadratic loss), mean absolute error function, smooth mean absolute error function, log-cosh loss function, and quantile loss function.

The optimizer 3426 adjusts the model parameters 3422 to minimize the loss function during training of the algorithm 3416. In other words, the optimizer 3426 uses the loss function generated by the loss function engine 3424 as a guide to determine what model parameters lead to the most accurate AI model. Examples of optimizers include Gradient Descent (GD), Adaptive Gradient Algorithm (AdaGrad), Adaptive Moment Estimation (Adam), Root Mean Square Propagation (RMSprop), Radial Base Function (RBF) and Limited-memory BFGS (L-BFGS). The type of optimizer 3426 used may be determined based on the type of model structure 3420 and the size of data and the computing resources available in the data layer 3402.

The regularization engine 3428 executes regularization operations. Regularization is a technique that prevents over- and under-fitting of the AI model 3430. Overfitting occurs when the algorithm 3416 is overly complex and too adapted to the training data, which can result in poor performance of the AI model 3430. Underfitting occurs when the algorithm 3416 is unable to recognize even basic patterns from the training data such that it cannot perform well on training data or on validation data. The optimizer 3426 can apply one or more regularization techniques to fit the algorithm 3416 to the training data properly, which helps constraint the resulting AI model 3430 and improves its ability for generalized application. Examples of regularization techniques include lasso (L1) regularization, ridge (L2) regularization, and elastic (L1 and L2 regularization).

The application layer 3408 describes how the AI system 3400 is used to solve problems or perform tasks. In an example implementation, the application layer 3408 can include software implemented on the system 150 or 300B. For instance, the application layer 3408 may correspond to the interface logic of the Verification Rendering Engine 460 of FIG. 4, which interprets the outputs of the AI Model 3430 (e.g., compatibility indicators and coordinate maps) to generate the interactive verification overlays for the user.

FIG. 35 (formerly FIG. 23) is a block diagram that illustrates an example of a computer system in which at least some operations described herein can be implemented. Components of the computer system 3500 can be used to implement the system 150, 300B, or the Centralized Processing Hub 400 of FIG. 4.

As shown, the computer system 3500 can include: one or more processors 3502, main memory 3506, non-volatile memory 3510, a network interface device 3512, video display device 3518, an input/output device 3520, a control device 3522 (e.g., keyboard and pointing device), a drive unit 3524 that includes a storage medium 3526, and a signal generation device 3530 that are communicatively connected to a bus 3516. The bus 3516 represents one or more physical buses and/or point-to-point connections that are connected by appropriate bridges, adapters, or controllers. Various common components (e.g., cache memory) are omitted from FIG. 35 for brevity. Instead, the computer system 3500 is intended to illustrate a hardware device on which components illustrated or described relative to the examples of the figures and any other components described in this specification can be implemented.

The computer system 3500 can take any suitable physical form. For example, the computer system 3500 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computer system 3500. In some implementations, the computer system 3500 can be an embedded computer system, a system-on-chip (SOC), a single-board computer system (SBC) or a distributed system such as a mesh of computer systems or include one or more cloud components in one or more networks. Where appropriate, one or more computer systems 3500 can perform operations in real-time, near real-time, or in batch mode.

The network interface device 3512 enables the computer system 3500 to mediate data in a network 3514 with an entity that is external to the computer system 3500 through any communication protocol supported by the computer system 3500 and the external entity. For example, the Data Ingestion Engine 410 of FIG. 4 utilizes the network interface device 3512 to receive data packages from source nodes 110 of FIGS. 1A and 1B. Examples of the network interface device 3512 include a network adaptor card, a wireless network interface card, a router, an access point, a wireless router, a switch, a multilayer switch, a protocol converter, a gateway, a bridge, bridge router, a hub, a digital media receiver, and/or a repeater, as well as all wireless elements noted herein.

The memory (e.g., main memory 3506, non-volatile memory 3510, machine-readable medium 3526) can be local, remote, or distributed. Although shown as a single medium, the machine-readable medium 3526 can include multiple media (e.g., a centralized/distributed database and/or associated caches and servers) that store one or more sets of instructions 3528. The machine-readable (storage) medium 3526 can include any medium that is capable of storing, encoding, or carrying a set of instructions for execution by the computer system 3500. The machine-readable medium 3526 can be non-transitory or include a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory devices 3510, removable flash memory, hard disk drives, optical disks, and transmission-type media such as digital and analog communication links. The Vector Store Index 434 and Node Capability Specifications 442 of FIG. 4 may be stored on such media.

In general, the routines executed to implement examples herein can be implemented as part of an operating system or a specific application, component, program, object, module, or sequence of instructions (collectively referred to as “computer programs”). The computer programs typically include one or more instructions (e.g., instructions 3504, 3508, 3528) set at various times in various memory and storage devices in computing device(s). When read and executed by the processor 3502, the instruction(s) cause the computer system 3500 to perform operations to execute elements involving the various aspects of the disclosure, e.g., the Candidate Matching Engine 440 or Verification Rendering Engine 460 of FIG. 4.

### EXAMPLES

The following examples are provided for illustration purposes and not intended to be limiting.

### Example 1. UI and an Explanation of the Workflow in an Example Use Case of Patient Referral

### Referral Intake.

Referrals can either be received through integrations with referring systems (e.g., NaviHealth, CarePort, etc.) or be manually entered by a user. If the referral is received via an integration the intake data is automatically populated. Each referral includes information in multiple categories, including:
General—basic demographic information
Insurance—primary, secondary, and tertiary insurance
Authorization—authorization tracking form
Referral—referrals source information (e.g., hospital, admit date, anticipated discharge date, etc.)
History/Physical—H&P notes
Medications—medication information
Other—miscellaneous information
Emergency Contacts—emergency contact information
Attachments—uploaded PDF/JPG/PNG documents with additional information

Once received, referrals populate a list of referrals at the patient referral level before starting the intake review process.

A referral may be “locked” while being reviewed by another user. This prevents other users, without requisite permissions, from editing answers to clinical questions while being reviewed.

A referral detail may show an overview section including each of the referred communities along with the status (e.g., Intake Review, Intake Submitted, Intake Denial Pending, Clinical Review, Clinical Submitted, Clinical Denial Pending, Clinical Denial Overturned, etc.)

### Central Intake/Intake Review.

Each referral intake is done including completing a sex offender check is completed and verifying insurance. The Sex Offender check integrates with a 3^{rd} party system via an API. Referral details are sent, and potential matches are returned and displayed, which can be reviewed by the user and confirmed by selecting a match or “no matches found” and submitting. Similarly, insurance verification integrates with a 3^{rd} party system (e.g., Waystar) via an API to identify an insurance verification match. Each service code can be selected to display a detailed description for each service.

After intake review is completed, the referral can be submitted or denied along with user entered notes. Denied referrals can be overturned by a denial review user.

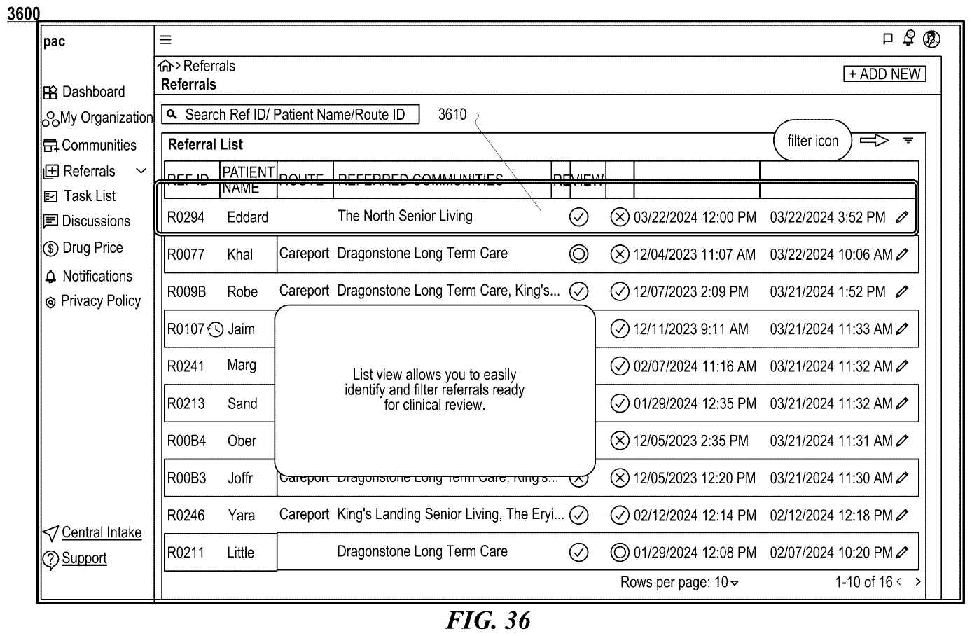

Clinical Review. See FIG. 36.

FIG. 36 illustrates an exemplary user interface 3600 for clinical review. Once a referral is submitted for an intake review, a community or central intake user accesses this interface to manage the queue. A specific patient referral may be identified and selected for review, as indicated by row 3610. The interface allows users to identify and filter referrals ready for clinical review based on status (e.g., “Clinical Review,” “Clinical Submitted,” or “Clinical Denial Overturned”). Additionally, the referral list may be sorted using a prioritization scoring method to highlight high-priority cases.

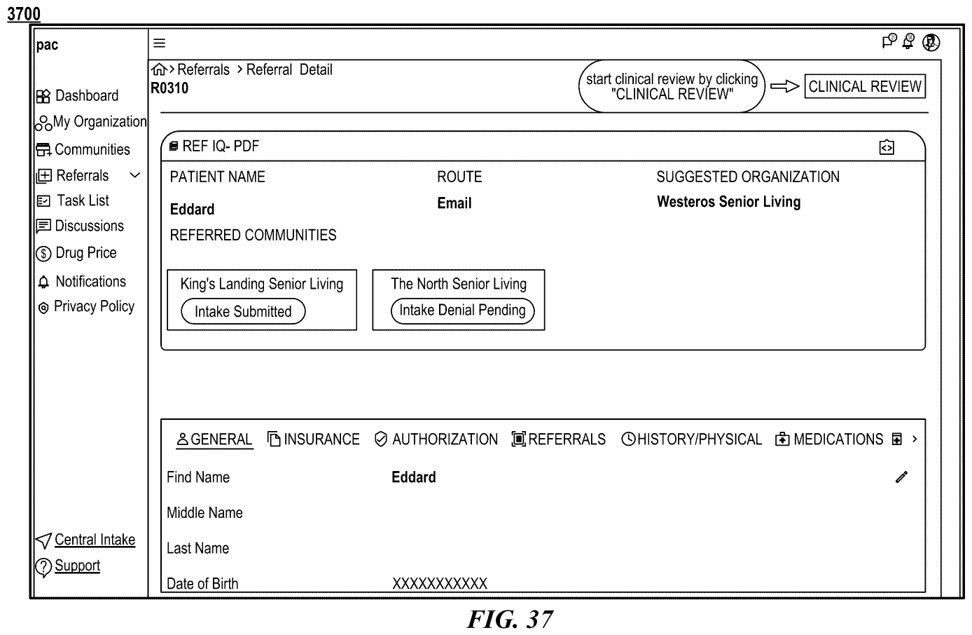

Each referral has a referral detail view (see exemplary user interface 3700 in FIG. 37) showing all information related to the specific referral, with specific tabs for different areas of information. This also shows each of the referred communities for the referral, along with a status for each of the communities. From this view, a user can start a clinical review.

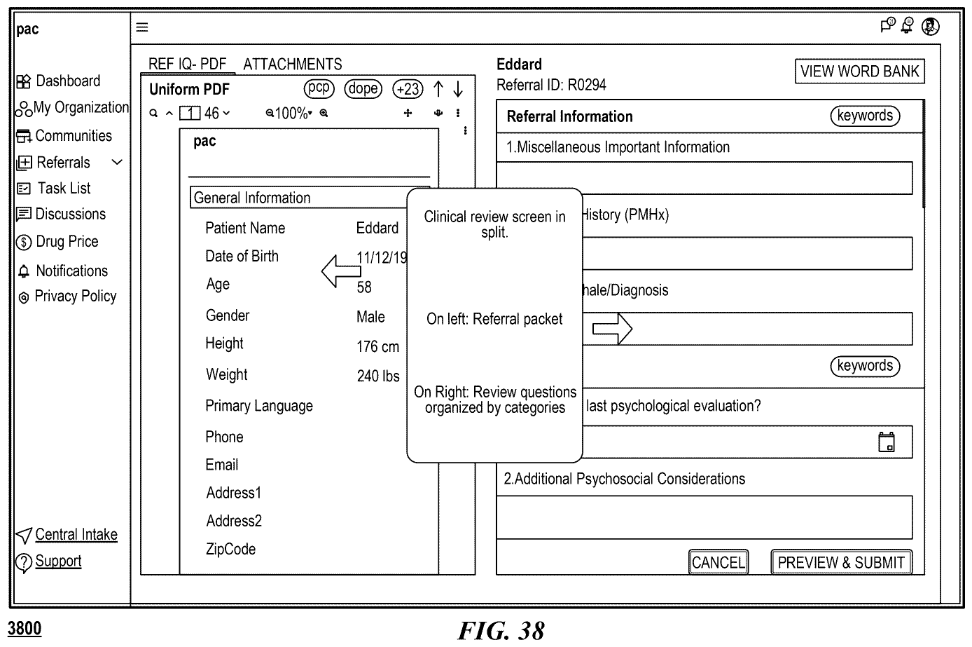

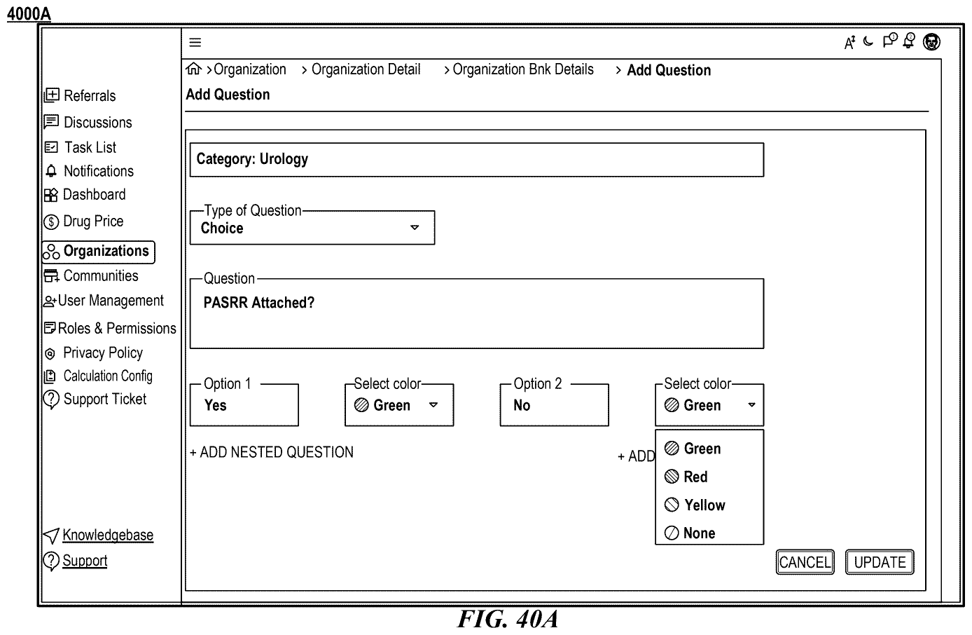

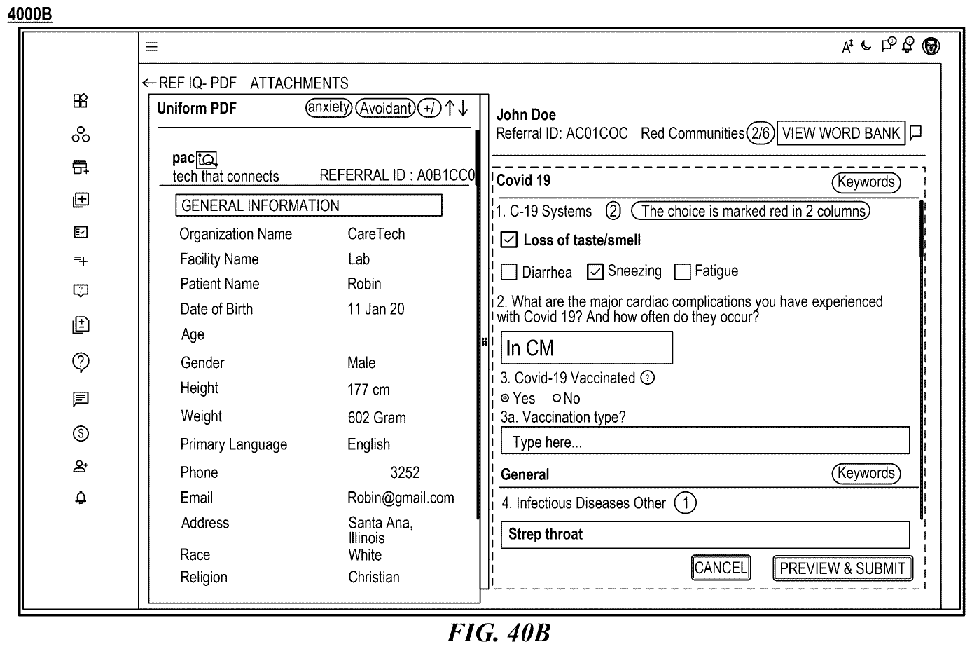

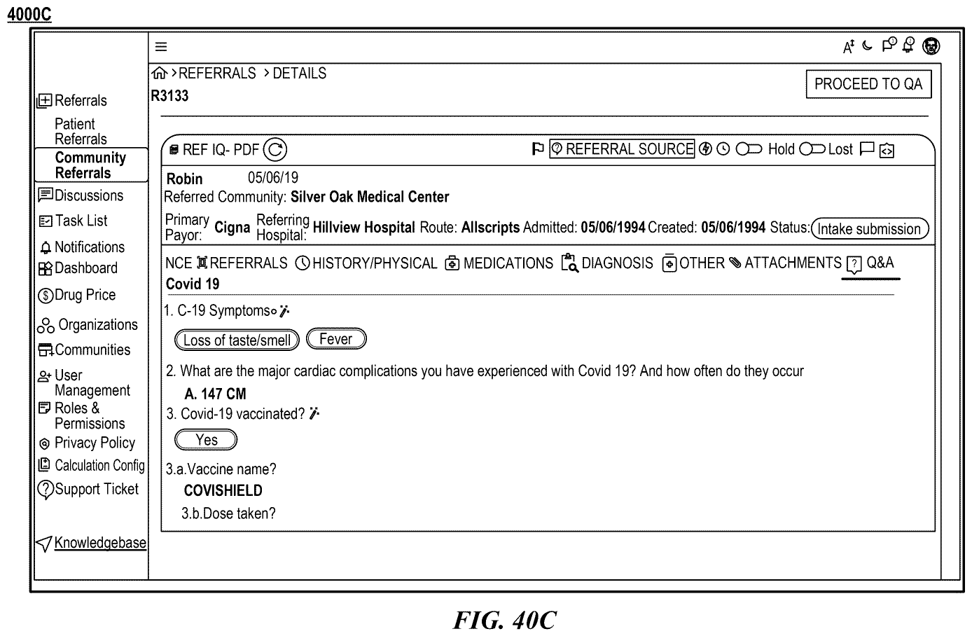

As illustrated in the exemplary user interface 3800 in FIG. 38, the clinical review for a specific referral is shown in a split screen with a PDF of the referral packet on one side and the clinical review questions organized by categories on the left (see also FIG. 40B). The questions need to be answered as part of the clinical review using the information from the referral packet. The questions are customized based on the organization's configurations. Each community may also have different and/or additional questions.

Keyword Highlighting. The referral packet is processed by OCR and keywords are highlighted. Each keyword is associated with a category corresponding to a category of the clinical review. The highlighted keywords allow the reviewing user to navigate to easily identify keywords related to a question or category of question, as well as to navigate to specific keywords one at a time. The keywords that are highlighted can be selective highlighted using the “word bank” depending on the clinical review. The keywords in the word bank are organized by keyword categories. Initial categories and words are provided and can be manually customized and curated by the organization, community users. Fuzzy matching logic is used to match keywords directly with words in the referral packet text. A default set of keywords is provided depending on the type of community and clinical questions. Keywords can be customized and added by organizations and communities to fit their clinical questions.

An LLM or NLP process may be used to identify words or sections that are relevant to keywords contextually. For example, passing a prompt along with text from the referral packet, where the prompt indicates identifying and returning a list of words or segmented text chunk from the text that either matches, synonymous, or are semantically relevant to a list of keywords for a particular category. The returned list of words and segmented text chunks can then be highlighted in the referral packet for review by the user. This can help to highlight relevant text related to a category that are not direct key words matches; instead highlighting logically relevant sections based on a combination of the key words. In simpler applications, it can identify and highlight various keyword synonyms.

LLM for Automated Clinical Review Question Answering. A LLM model is used to populate answers to clinical review questions that are then reviewed and edited by the reviewing user. Each prompt includes instructions to act as a referral reviewer, a specific clinical question, extracted referral text data. The LLM may also be provided community information, e.g., community capabilities, for further context. The prompt may also include indication of input data format, and indication of requested output data format. Each question is paired with a prompt that is tailored so the LLM provides a response with a proper answer-type (e.g., select between specified options) and formatting for properly answering the clinical question.

The LLM model can be fine-tuned to provide better answers to the clinical review questions by using a pre-trained model and either retraining layers or training additional layers using training referral packets and corresponding clinical review questions and human edited and/or approved answers. In addition, few-shot prompting may be used to improve answers, by providing in the prompt example inputs and output answers.

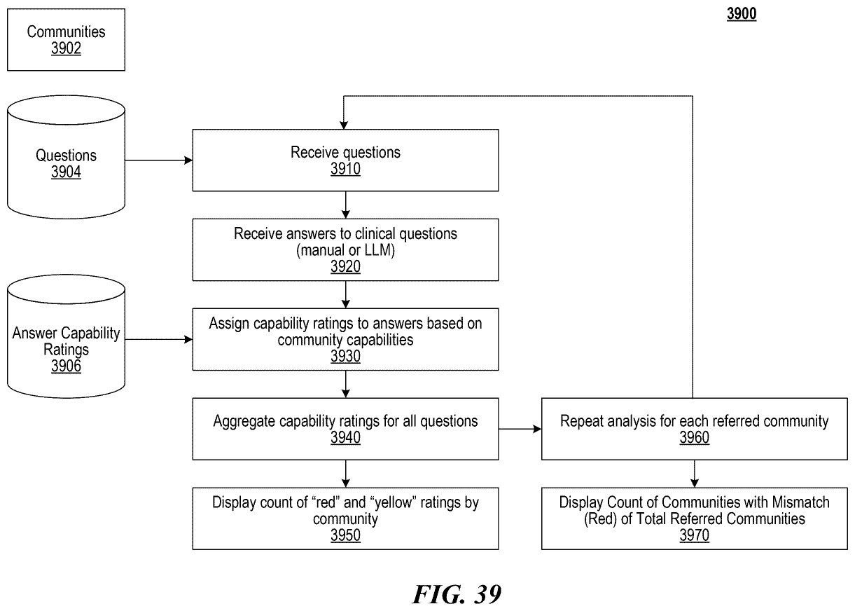

LLM Answer Citations. The LLM model can provide the user one or more citations to sections of the referral packet (either as verbatim quoted text or a link to specific text) used for generating the answer to the clinical question. The reviewer can then review and verify the LLM generated answer. For example, as shown in FIG. 39, this can be done using a RAG (retrieval augmented generation) method that breaks up the referral packet text into smaller chunks. The smaller chunks that are most pertinent to the prompt and question can then be identified and ranked by using an embedding-search method. The most pertinent chunks may be the only chunks that are provided as the context to the LLM to answer the question and then provided to the user as citations. Alternatively, the whole text may still be provided as context to the LLM, but the most pertinent chunks identified may still be provided as citations to the user.

FIG. 39 illustrates a process for capability matching between referral needs and community capabilities, according to some embodiments of the present technology. The process 3900 utilizes data from communities 3902, a question database 3904, and answer capability ratings 3906.

The process 3900 begins by receiving questions at 3910 for clinical review. The system then receives answers to these clinical questions 3920, which may be generated either manually or through the LLM-based process described in FIGS. 7, 8, 10, and 11. For LLM-generated answers, the system can evaluate responses and assign capability ratings based on community specifications.

At 3930, the system assigns capability ratings to answers based on community capabilities. Each answer receives a rating (e.g., green, yellow, or red) indicating the level of compatibility with a community's capabilities. The ratings are assigned by comparing answer content with predefined capability specifications stored in the answer capability ratings database 3906.

The process 3900 includes aggregating capability ratings for all questions at 3940. This aggregation creates a comprehensive capability assessment for each community. At 3950, the process 3900 includes displaying the count of “red” and “yellow” ratings by community, providing a quick visual indication of potential compatibility issues.

The process 3900 includes repeating at 3960 for each referred community in the network. Finally, at 3970, the process 3900 includes displaying a summary count showing the number of communities with capability mismatches (red ratings) out of the total number of referred communities.

FIG. 40A is a view 4000A of a clinical question editing including selecting capability rating color for each answer option. FIG. 40B is a view 4000B of clinical review showing capability rating icons adjacent questions, and an alert showing a count of “red communities.” FIG. 40C is a view 4000C of a community review showing a capability rating (red) icon adjacent to a question.

Submit Completed Clinical Review. After finalizing the clinical review questions, the clinical review is submitted or denied. If submitted, it can then be reviewed by each community. Additional community specific questions may need to be answered. Then, the referral can be submitted or denied by each community.

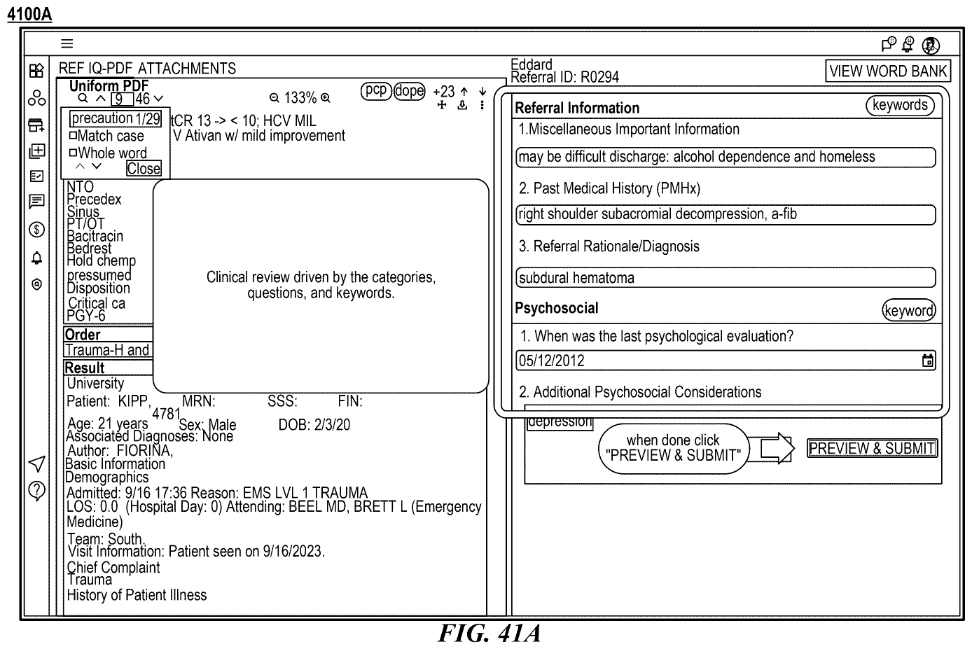

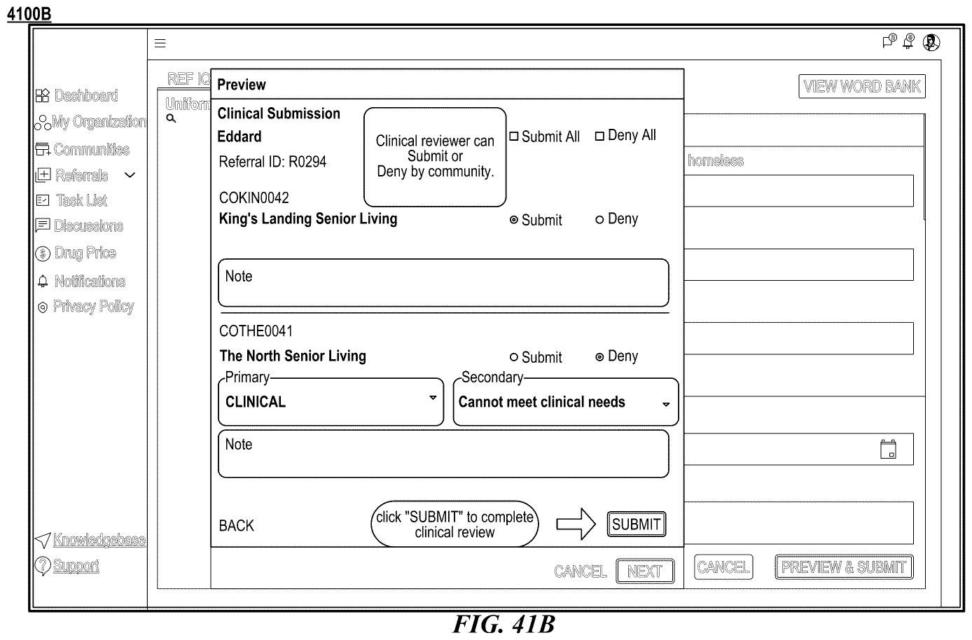

FIG. 41A is a view 4100A of a clinical review showing a referral packet and clinical review questions. FIG. 41B is a view 4100B of a clinical review submission modal illustrating a submission and denial for two different communities.

Additional Community Referrals. The clinical review can be completed by a user at one community, and questions common between another community may be pre-populated for the second community. Questions that are specific to each community may still need to be completed. In addition, if later a newly referred community within the organization comes in from a referral source, the questions common between the newly referred community and the prior referred communities may be prepopulated. Common questions may be based on a default set of questions, or an organization that uses a set of questions that can be assigned to different communities, of which many may be common.

As discussed further below, a recommendation for another community (not initially referred by the referral source) that potentially is a better fit may be automatically determined based on capability matching. The recommendation is then automatically added to and sent along with the denial message to the referral source when denying a referral. For example, a recommended community may be included in a notes field along with reasoning for the rejection, and reasoning for the recommendation for another community (e.g., based on specific needs of the referral and corresponding capabilities of the denied community and recommended community).

Similarly, this can be done based not only on community capabilities but also case mix of communities. For example, a community may deny a referral not due to a particular capability mismatch but rather based on clinical complexities that can technically be handled by the community. For example, due to limited capacity for certain types of cases. Another community may have more bandwidth for this type of case and would be a better fit and could be recommended back to the referral source. More specifically, this could be done by tracking an actual count and maximum count for certain types of cases at a facility, which can be compared against the answers to clinical questions (e.g., yellow) to determine if the referral would or would not be a good fit.

### Community Review.

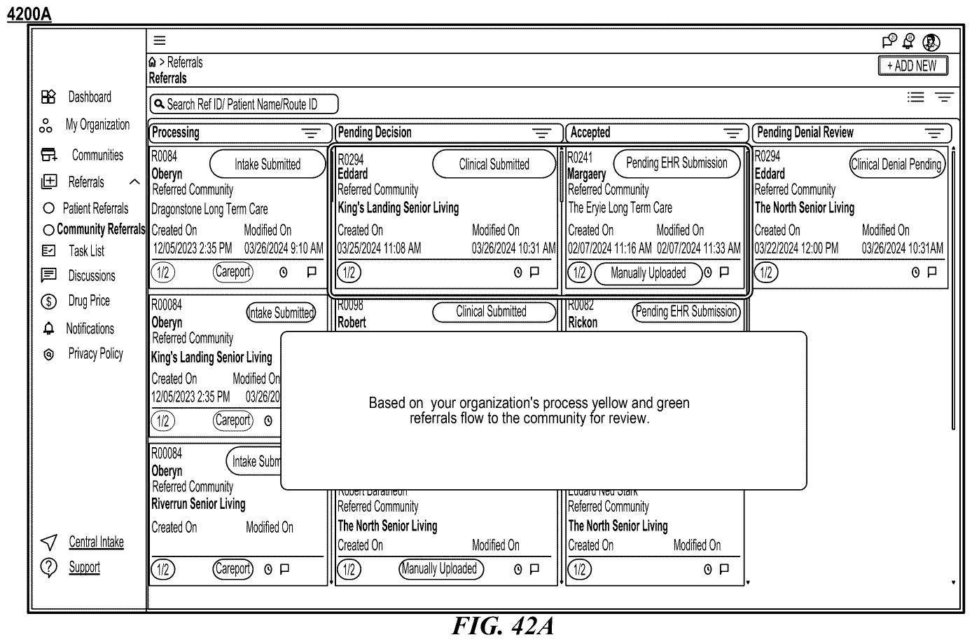

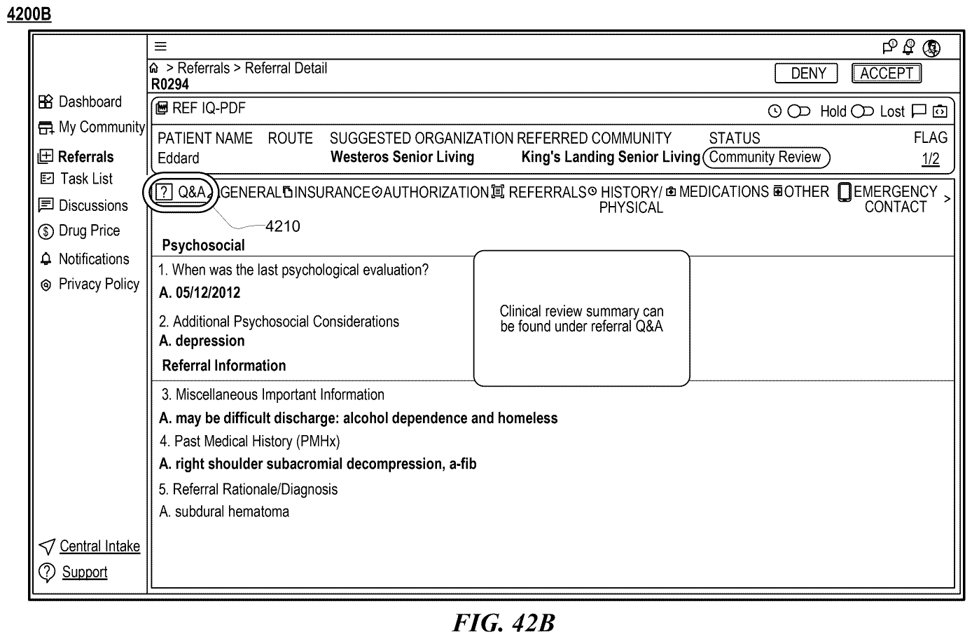

After clinical review, a community review can be completed by a community review user. This may include reviewing the referral including the completed intake review and clinical review answers. A community review user can see a clinical review summary under a referral Q&A tab (see 4210 in exemplary user interface 4200B in FIG. 42B).

As illustrated in the exemplary user interface 4200A in FIG. 42A, the community review workflow can be shown in grid view flowing left to right including columns related to status (e.g., Processing, Pending Decision, Accepted, and Pending Denial Review). Each referral is shown in a card with a summary of referral information including the referral's name, status, referred community, creation and modification date-times. It can also show a tag indicating the referral channel/source (e.g., CarePort, NaviHealth, Manually Uploaded, etc.). Another tag can indicate how many intake review checks have been completed between sex offender check and insurance verification (e.g., “0/2”, “1/2”, “2/2”). The clock icon can be selected to open logs, including messages related reasoning for overturning a denial from a denial management user. The message icon opens a HIPAA compliant messaging application (Sendbird) between users at the community and organization related to the specific referral. This provides a quick, safe, and compliant way to communicate about a specific referral amongst users. Although not shown, other icons may indicate a patient was added to an org wide high-risk admit list (discussed below), other dynamic indicators may show the number of communities that have a red capability mismatch out of the total number of referred communities (e.g., “2/5”).

Each of the statuses is given a specific color related to where it falls in the process. The following is the meaning of the color of each of the statuses:
Green: Simply, the referral is early in the process and review steps need to be taken.
Yellow: The referral has been accepted but there are additional steps that need to be done in order to fully process the referral (e.g., the referral is pending EHR submission).
Red: The referral has been denied, and may need to be reviewed for final rejection by denial reviewer. This may be a denial at the clinical review, community review, or even the initial intake review.

### Community Acceptance.

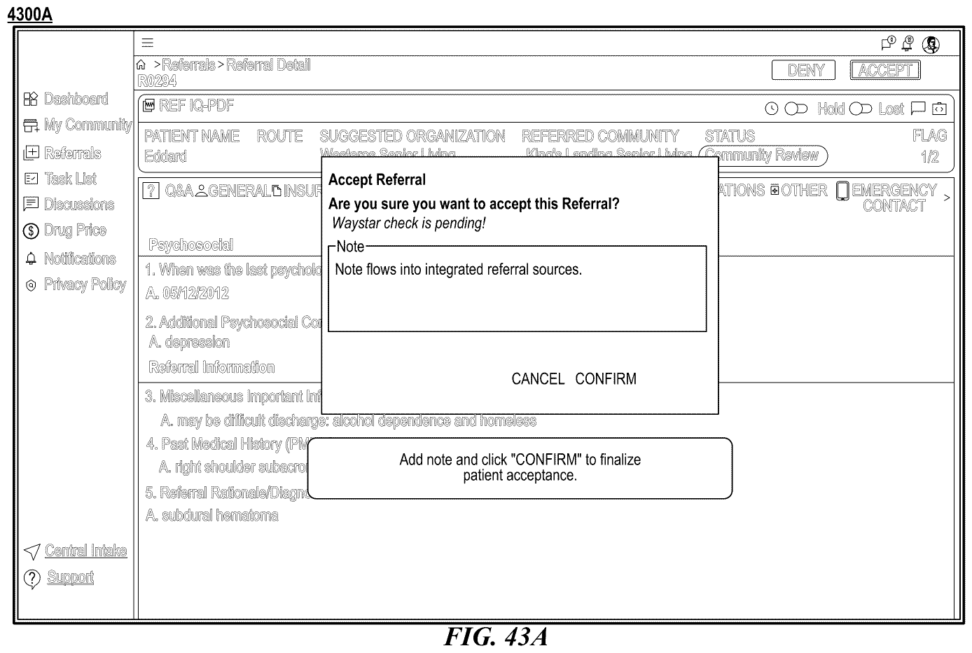

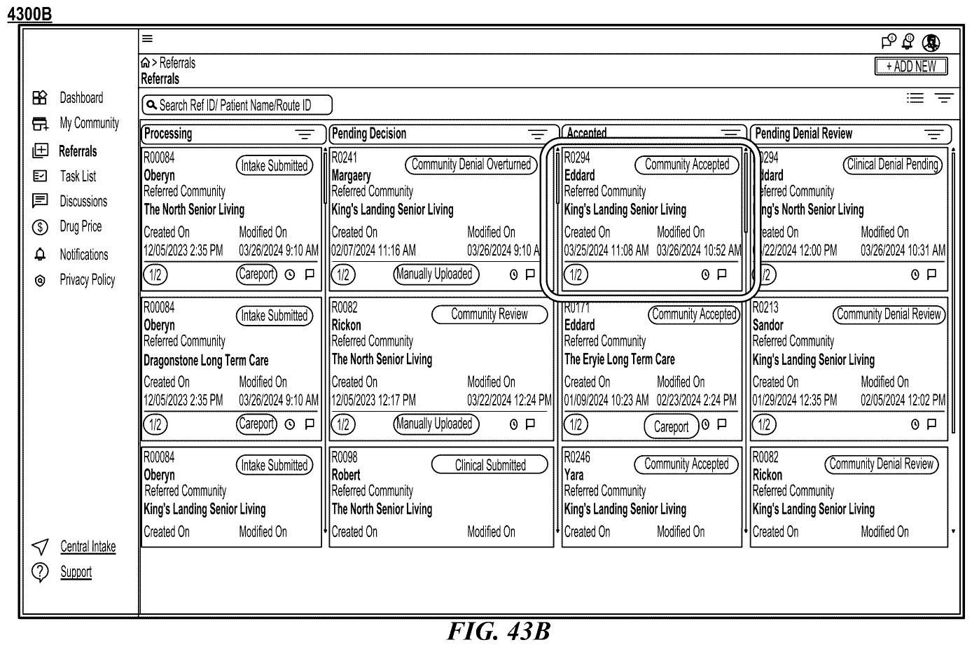

After completion of a community review of a referral, the referral can be accepted or denied. FIG. 43A shows a view 4300A of a referral accepted by a community. FIG. 43B shows a view 4300B of a referral accepted by community shown in grid view of referrals.

A user can enter notes when accepting or denying the referral. Warning messages can be shown to the user in the modal, for example, if an intake review check has not been completed (e.g., insurance verification via Waystar).

If the referral is accepted by the community user, the status may be shown as “Community Accepted” and can create a pending patient record or match to an existing record in an EMR/EHR (e.g., PCC). If the referral is denied, the status may be shown as “Community Denial Pending”, which may be overturned by a denial management user, which may return the referral to the prior status. The denial management user can modify answers to questions before it is sent back to the prior status.

“Hard Stop” Rejection Alert Message. An alert banner message may be displayed at the top of the referral based on received data or data taken from documents via OCR (e.g., FIGS. 40B and 40C). The alert message indicates that the review should stop because of a potential “hard stop” rejection, with an explanation to the user. Referral patient need data may be compared to capability data and/or rules for each community in the organization. As an example, if a community does not have a ventilator and the patient needs a ventilator; this would indicate that no further review is needed, and the referral should be rejected. The criteria for the hard stop are defined by the community and are modifiable as desired by the administrative user of the community.

When determining if there are any potential “hard stops”, available data is checked before looking at other data (e.g., data extracted from referral documents via OCR).

Alternatively, if only a portion of referred communities for an organization are affected by patient needs, the message may be displayed only in relation to the specific communities when submitting the clinical review to help guide the user in denying for only the community that lacks the needed capability.

High Risk Patient List. One of the potential alert messages can be related to a table of high-risk patients that is maintained across the organization. Patients are added to the list by specific communities along with the reason why. If they are later referred to another community in the organization, this may trigger an alert message to the reviewer indicating the referral is on the high-risk list. High-risk patients are identified by comparing the name and birthday date of the patient to corresponding fields of each patient in the high-risk patients table for the organization. It also includes the community that added the patient to the high-risk patient table along with the high-risk admit reason.

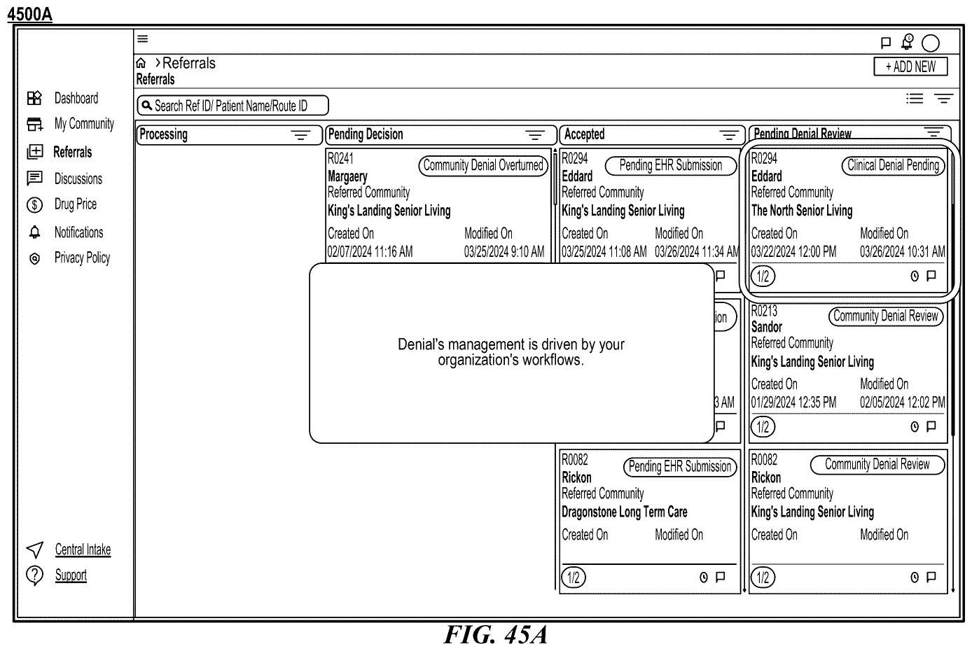

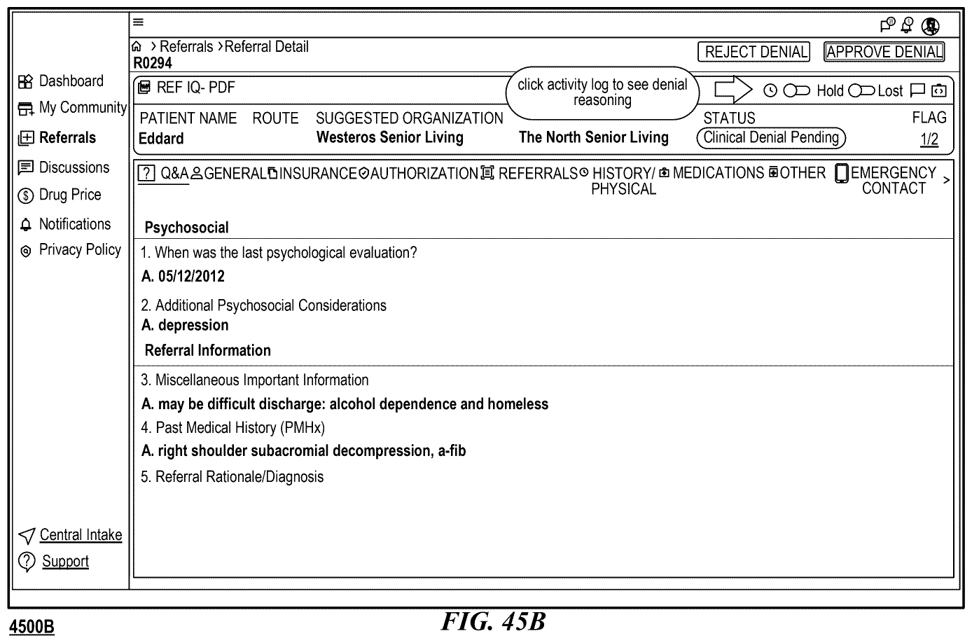

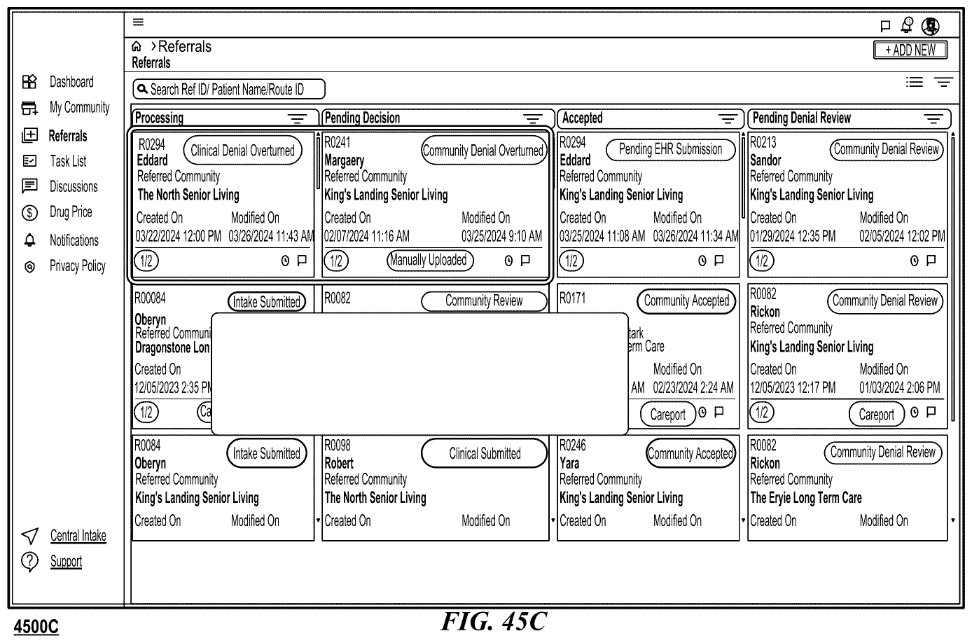

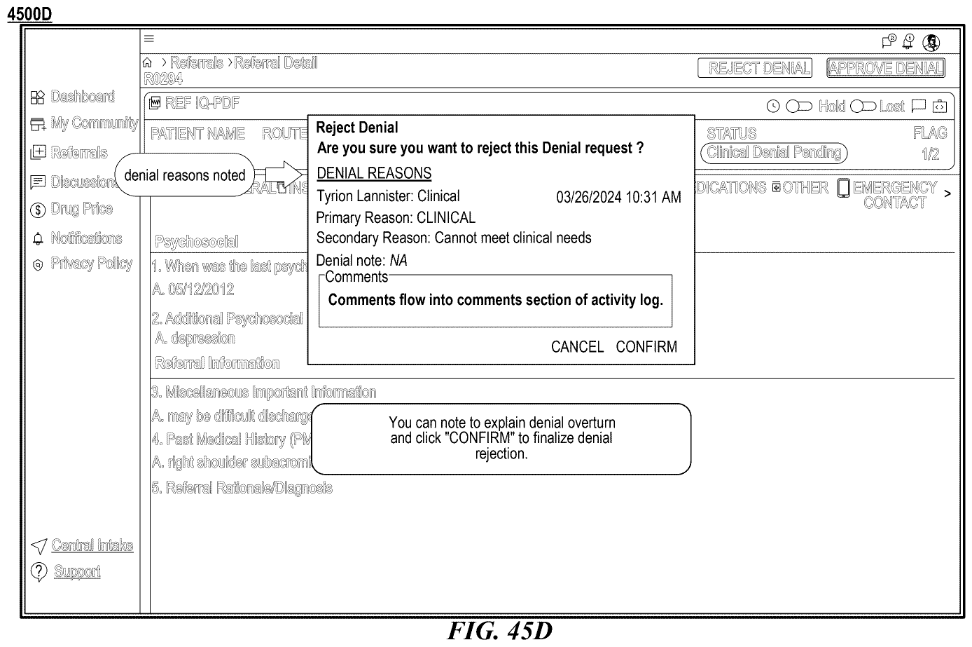

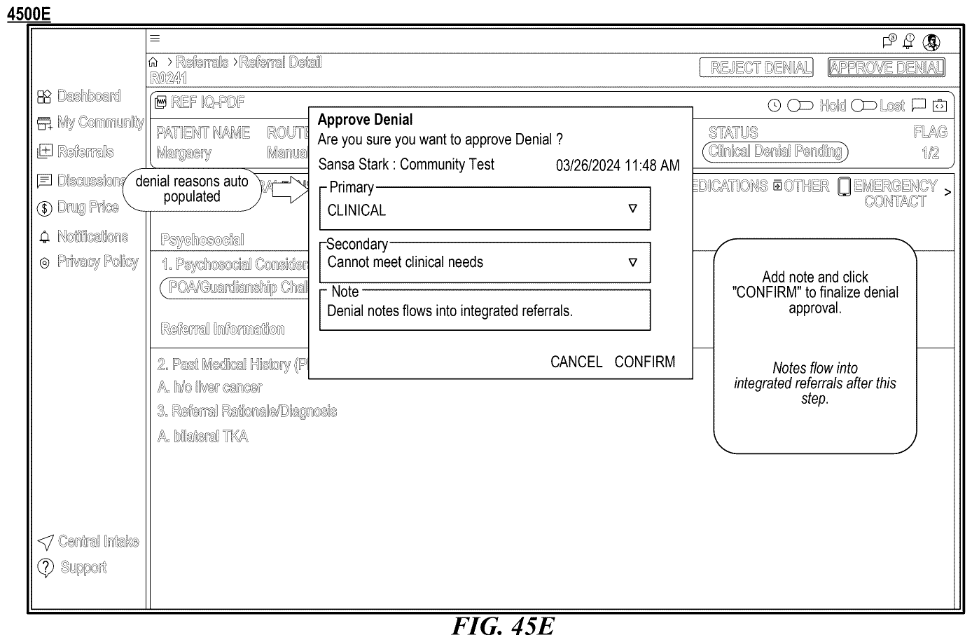

The alert is displayed as a banner at the top of the specific referral page (see exemplary user interface 4400 in FIG. 44). It may also be shown as a specific icon on the bottom of the referral card in the grid view (see exemplary user interface 4500A in FIG. 45A). A high-risk referral may also be accounted for in referral prioritization calculations. For example, adjusting the score lower by some factor so that it is deprioritized.

### Patient Acceptance.

After the patient accepts, the user initiates patient admit process causing an EMR/EHR integration (e.g., PCC) to search for patient record matches. This allows patients records to be merged if matched. Non-matched patients may have a new patient record created. In the EMR/EHR, the patient can be accepted and added to waitlist per normal workflow.

### Denial Management.

A denial management user has full access to referrals and can review pending denials. This includes seeing denial reasoning entered by the user that initially denied the referral. FIG. 45B shows a denial management user view 4500B of specific referral being reviewed.

Rejecting Denials. The denial management user can reject the denial, which may return it to wherever the referral was denied in the workflow. For example, if denied at community level referral it may return to community workflow. The status may indicate that the denial was overturned. When selecting to reject the denial a modal is displayed allowing the user to explain why the denial was overturned. FIG. 45C is a grid view 4500C showing referrals that have had denials overturned returned to workflow.

Approving Denials. The denial management user may approve a denial after reviewing it, which updates the denial status to denial approved. This may remove the referral from grid view but are still searchable. The approved denial can also be reverted if needed. When selecting “Approve Denial”, a modal is displayed that allows the user to add notes for why the denial was approved before the user confirms. These notes are included in an automated message to the integrated referral source. This includes primary and secondary denial rationale, which specifies the rationale group and more specific rationale (e.g., rationale groups: “Bed Availability”, “Business”, “Service”, “Clinical”, “Financial”, “Staffing”). FIGS. 45D and 45E are views 4500D and 4500E showing modal for entering explanation and confirming denial rejection.

Additional Referral Community Recommendation. In some cases, the referral may be rejected for the referred community within an organization due to patient needs that cannot be met by the referred community. However, there may be another community or communities within the organization that the system identifies as potentially being a good fit and meeting the needs of the referral patient. Accordingly, the system may suggest including, or automatically add, in the denial message sent to the referral source an indication of community or communities within the organization that potentially could be a good fit along with reasoning. For example, the message can indicate that the initially referred community does not have one or more needed capabilities, but the recommended community does have the needed capabilities. The referral source may in response submit a new referral for this community. Note: the PHI of the referral cannot be shared directly with another community in the organization, thus making this a critical piece.

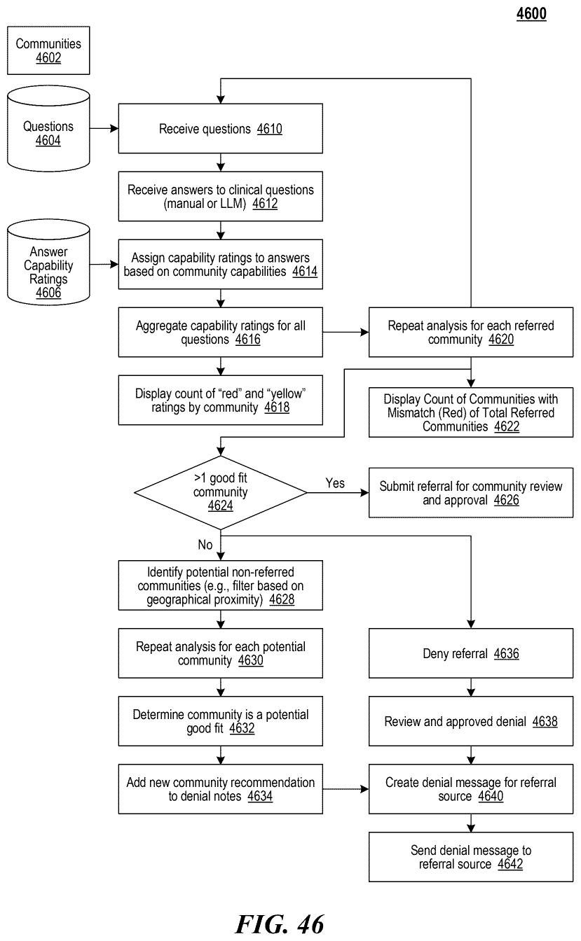

FIG. 46 shows a process for identifying and recommending suitable communities for referrals, according to some embodiments of the present technology. The process begins by utilizing data from communities 4602, questions database 4604, and answer capability ratings 4606 to process referrals and assess compatibility.

Operations 4610-4622 mirror the capability matching process described earlier: receiving questions (4610), obtaining answers through manual input or LLM processing (4612), assigning capability ratings (4614), aggregating ratings (4616), displaying ratings by community (4618), repeating analysis for each referred community (4620), and displaying the overall community mismatch count (4622). For example, the number of mismatched communities of the total referred communities may be displayed, or the number of capability issues for the referral by community may be displayed (e.g., “2 red”, “3 yellow”, etc.).

At decision point 4624, the process determines if there is at least one good fit community. If yes, the referral is submitted for community review and approval (4626).

If no, the process identifies potential non-referred communities (4628). As part of the denial message that is sent back to the referral source (e.g., hospital), a recommended community within the organization is added along with reason. The recommended community or communities is identified by first identifying potential communities. Potential communities can be identified based on geographical proximity, type of facility, capacity, case mix of community, among other factors. Geographical proximity can be based on ZIP code, distance to initial referred communities, or an association between the initially referred communities and other communities in an organization (e.g., an organization can group or link communities together that can be considered together).

The process can then compare patient needs (e.g., question answers) with the capabilities of each potential community in the organization. Based on the answers to clinical questions, a capability rating is determined for each answer for each potential community. The process 4600 repeats the analysis for each potential community (4630) and determines if a community is a potential good fit (4632). As a simplified example, if the question “Needs a ventilator?” is answered “yes”, it may be determined to not align with the capability of the referred communities (e.g., given capability rating of “red” for the referred community); however, this same answer does align with capability of the referred communities (e.g., given capability rating of “green” or “yellow” for the potential community).

When a suitable alternative is identified, the system adds the new community recommendation to denial notes (4634), denies the referral (4636), reviews and approves the denial (4638), creates a denial message for the referral source (4640), and sends the denial message (4642).

As previously discussed, once the new community-specific referral is received, the intake review checks may already be completed and clinical review questions that are common between communities within an organization may be pre-populated with prior answers. Accordingly, in completing the clinical review for the newly referred community only new community-specific questions may need to be answered and pre-populated answers be reviewed.

### Analytics Dashboards

The system (e.g., system 150, 300B, 400, 3400) includes dashboards that can visually show data for the organization and specific communities to help answer important questions to make improvements and address issues. For example:
How many minutes does each step take?
How many referrals were won?
What percentage of each denial reason?
What sites struggle with accepting clinically complex patients?
What users are the most active?
Where should business development effort be focused?

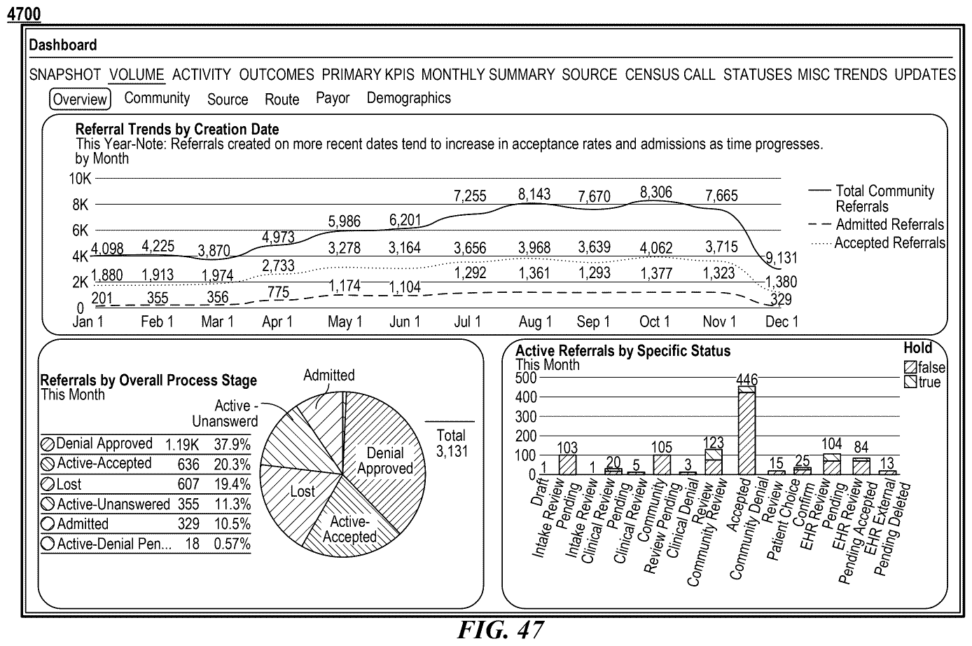

FIG. 47 is a view 4700 of dashboard overview, according to some embodiments of the present technology. Time-based referral notification (e.g., after a predetermined time passed, trigger an alert may indicate referral is likely not going to be won. The threshold may be dependent on factors including area. If certain questions take longer to handle/process (for clinical review), an alert message may be triggered with a recommendation for creating a new team to handle one or more questions.

FIGS. 48A-48C are various views 4800A-4800C of a patient referral workflow, according to some embodiments of the present technology. FIG. 48A shows a role-defined intuitive user interface (UI) 4800A. The interface implements an intuitive design that optimizes user experience through clear information hierarchy and logical workflow progression. Users can take direct actions through workflow elements.

The system disclosed herein implements standardized data structures to process referrals consistently across multiple input sources. This standardization enables uniform handling of information regardless of whether it comes through direct system integration, manual entry, or other input channels. As illustrated in FIG. 48B, the interface 4800B of the system provides streamlined access to comprehensive information packages. A package includes structured summaries, relevant attachments, and supplementary analytical insights, all organized in a standardized format for efficient review and processing. The system supports team collaboration through an integrated communication platform. This referral-centric messaging system enables team members to discuss specific referrals, share insights, and coordinate actions while maintaining all communications in the context of the relevant referral data.

FIG. 48C illustrates a configuration interface 4800C for managing a “Word Bank” or keyword repository. This interface enables users to define and customize specific keywords (e.g., “Smoking,” “Aggressive,” “Depression”) associated with particular clinical categories (e.g., Psychology) or facilities. The interface allows the assignment of specific color codes to each keyword. These user-defined visual attributes are utilized by the system to automatically highlight corresponding terms within the unstructured text of referral documents (as described in the verification interface embodiments), thereby facilitating rapid visual identification of critical risks or patient attributes during the review process. The interface supports adding, removing, and configuring these keywords to tailor the system's highlighting logic to the specific needs of the organization.

The system provides comprehensive user management capabilities, supporting unlimited user accounts with differentiated user types and roles. Each user type has specific permissions and access levels within the system.

The system maintains databases of configurable elements including keyword and phrase collections for content analysis and search operations. It supports detailed profile management for service providers and resource allocation entities, along with integrated calculation tools for cost analysis.

The system implements structured query management through configurable question sets and category organization. These elements can be customized based on organizational requirements and processing needs. A notification system keeps users informed of relevant events and status changes throughout the processing workflow.

These components are integrated into the centralized architecture, enabling consistent data access and standardized operations while maintaining appropriate access controls based on user roles. The system allows flexible configuration of these elements while preserving processing standardization.

The system provides a comprehensive analytics dashboard that enables immediate access to system metrics and performance data, as illustrated in the exemplary user interface 4900 in FIG. 49. The dashboard presents multiple data views through an intuitive interface, integrating both summary statistics and detailed analytical breakdowns.

The main interface includes a navigation sidebar providing access to key system functions including organization management, community settings, referral tracking, discussions, user management, and role-based permissions. The primary display area shows key performance indicators through various visualization methods including line graphs, pie charts, and numerical summaries.

The dashboard enables data analysis across multiple dimensions through tabbed navigation showing primary key performance indicators (KPIs), monthly summaries, source analysis, and detailed breakdowns. The visualizations track metrics such as referral volumes, processing times, acceptance rates, and outcome distributions. Interactive elements allow users to explore specific aspects of the data, with both desktop and mobile-optimized views providing consistent access to analytics.

This analytics interface supports operational queries including processing volumes, resource utilization metrics, outcome tracking, and performance analysis. The system provides insights into processing efficiency, resource allocation, and outcome patterns while maintaining data consistency across different view formats.

Beyond patient referral, the technology can be adapted to various other domains while maintaining the same systematic approach through processes and systems disclosed herein including the process 200A/200B/200C and system 150, 300B, 400, or 3400. Additional examples include student support service referrals in educational settings, and qualified vendor referrals in supply chain management for both products (e.g., raw materials, equipment) and services (e.g., maintenance, logistics support).

### Example 2: Large File Chunking Internal Referral Structure Prompt

You're an assistant designed to provide information from the content provided by Users.
You are given two inputs: a chunk of text from a PDF document and a JSON object containing questions.
Your task is to summarize the content of the provided PDF chunk in such a way that the summary includes the necessary information to answer the questions in the JSON object. If the content contain History, Diagnosis list all of them and add the details of each of it elaborately.
If the text contains any medications mentioned list them all with the necessary details.

### Example 3: General Information Prompt

You're an assistant designed to provide information from the content provided by Users.
Users will provide a string of JSON object with “Type”, “value”, you'll respond with the JSON object by following the instructions given below:
provide answer for each question by replacing the “value” field for each object in JSON”,
Each questions answer should be structured according to its type (string, number, date) as specified.
Each questions should be answered according to the details provided in the “description” field.
For questions with an “options” field select the answers only from the list of the given options.
Provide dates in a YYYY-MM-DD format and time in UTC with values only.
For all phone numbers the format should be in xxx-xxx-xxxx.
If SSN is masked, keep the field empty.
Height should be in cm and Weight in lbs. Return the values without units for Height and Weight.
Answer the names in the format FirstName then MiddleName and LastName.
Fill the DOB field with date of birth only, and not a hospital admission date.
If not enough information found about the question leave the “Value” field empty (e.g., Value: “ ”).
Here's an example of your output format:
{
“EmergencyContacts”: [
{
“ContactNumber”: {
“Type”: “string”,
“Value”: “555-555-5555”
},
“Name”: {
“Type”: “string”,
“Value”: “John Smith”
},
“Relationship”: {
“Type”: “string”,
“Value”: “Brother”
}
}
],
“General”: {
“description”: “General demographics of the patient”,
“DOB”: {
“Type”: “date”,
“Value”: “1995-04-29”
},
“Gender”: {
“Options”: {
“Female”: 2,
“Male”: 1
},
“Type”: “number”,
“Value”: 1
},
“PatientAddress”: {
“Address1”: {
“Type”: “string”,
“Value”: “723 Bobs St.”
},
“Address2”: {
“Type”: “string”,
“Value”: “ ”
},
“City”: {
“Type”: “string”,
“Value”: “Waynesburg”
},
“StateCode”: {
“Type”: “string”,
“Value”: “GA”
},
“StateName”: {
“Type”: “string”,
“Value”: “Georgia”
},
“ZipCode”: {
“Type”: “number”,
“Value”: “12345”
}
},
“PhoneNumber”: {
“Type”: “number”,
“Value”: “555-428-5555”
}
},
“Insurance”: {
“description”: “Insurance details of the patient”,
“PrimaryFinancialClass”: {
“Type”: “string”,
“Value”: “Medicare”
},
“PrimaryGroupNumber”: {
“Type”: “string”,
“Value”: “123456789”
},
“PrimarylnsuranceClass”: {
“Options”: {
“ManagedCare”: 6,
“VeteranAffairs”: 7,
“HMOCommercial”: 3,
“Medicaid”: 2,
“Medicare”: 1,
“Private”: 4,
“Unknown”: 5
},
“Type”: “number”,
“Value”: 1
},
“PrimaryPlanDescription”: {
“Type”: “string”,
“Value”: “FREEDOM BLUE”
},
“PrimaryPlanNumber”: {
“Type”: “string”,
“Value”: “HRF45787454588”
},
“PrimaryPlanProviderAddress”: {
“Type”: “string”,
“Value”: “111 FIFTH AVENUE Waynesburg GA 12345”
},
“PrimaryPlanProviderContact”: {
“Type”: “number”,
“Value”: “555-555-5555”
},
“PrimaryPlanProviderName”: {
“Type”: “string”,
“Value”: “Medicare”
}
},
“Referral”: {
“description”: “Referral details”,
“AccountNumber”: {
“Type”: “string”,
“Value”: “BBB456789123”
},
“AdmissionDate”: {
“Type”: “date”,
“Value”: “2023-02-09”
},
“AdmittingPhysician”: {
“Type”: “string”,
“Value”: “DANIEL R Smith”
},
“AttendingPhysician”: {
“Type”: “string”,
“Value”: “Bill MAE East”
},
“DateFirstSent”: {
“Type”: “date”,
“Value”: “2024-01-04 14:49:25”
},
“DateRecentRevision”: {
“Type”: “date”,
“Value”: “2024-01-04 14:49:25”
},
“Facility”: {
“Type”: “string”,
“Value”: “UPMC ALTOONA MPAC”
},
“Hospital”: {
“Type”: “string”,
“Value”: “Wilkes-Barre General Hospital”
},
“LevelOfCare”: {
“Type”: “string”,
“Value”: “Ortho”
},
“Location”: {
“Type”: “string”,
“Value”: “555North River Street, Bobsville, GA 5555”
},
“MRN”: {
“Type”: “string”,
“Value”: “B56961”
},
“PatientType”: {
“Options”: {
“Bedhold”: 10,
“Emergency”: 8,
“InPatient”: 5,
“IRF”: 6,
“LongTermCare”: 2,
“Observation”: 7,
“Rehospitalization”: 9,
“ShortTermCare”: 1,
“ShortToLongTermCare”: 3,
“Unknown”: 4
},
“Type”: “number”,
“Value”: 3
},
“PrimaryCarePhysician”: {
“Type”: “string”,
“Value”: “MANISHA N Smith”
},
“ProjectedDischargeDate”: {
“Type”: “date”,
“Value”: “ ”
},
“ReferralSenderComment”: {
“Type”: “string”,
“Value”: “Ready for discharge. PASRR to follow”
},
“ReferralSenderName”: {
“Type”: “string”,
“Value”: “Karen Walker”
},
“ReferralSenderPhoneNumber”: {
“Type”: “number”,
“Value”: “ ”
},
“ReferringPhysician”: {
“Type”: “string”,
“Value”: “Sara Kannucheck Jones”
}
}
}

### Example 4—Diagnosis and History Prompt

You are an assistant designed to process medical data.
A text extract from a PDF (which could contain medical records, diagnoses, history, medications, and other details) will be provided to you as the user input. Alongside, you will receive a JSON template with various fields like Diagnosis, Medication, History, etc.
Your task is to update the JSON object based on the information found in the text context provided and return the same JSON by following the instructions given below:

### Instructions:

### 1. Understanding the Input:

Text Context (from PDF): You will receive a text extract from a PDF. This text will contain medical information (e.g., diagnosis, medications, test results, patient history). Your job is to read through this text and extract information to fill the corresponding fields in the provided JSON template.
JSON Template: The JSON object you receive will contain fields such as Diagnosis, History, Medication, etc. Your task is to update the Value field in each of these objects based on the details from the text.

### 2. Answering the JSON Fields:

### Diagnosis:

Extract all diagnosis or related test results from the text context.
There can be one or more Diagnosis, you should list each and everyone of them into the DiagnosisList.
For each diagnosis, include relevant details to the corresponding fields in the Diagnosis array in the JSON. If multiple diagnoses are mentioned, include them as separate objects in the array.

### History:

You should list each and every medical history and related details from the text context into the HistoryList.
For each history entry, include the Title, Order, Test, Date, and Result.
If the context includes patient history or past medical tests, include the whole details of it elaborately into the Result field.
If no history is provided, leave the field empty.

### Date and Time Handling:

Extract the date and time of each diagnosis and history result.

### Date Format:

Return the diagnosis and history date in strict YYYY-MM-DD format.

### Time Extraction:

If the time of diagnosis or history is mentioned along with the date, extract it.
Time can be in 12 hour or 24 hour format. If AM/PM is not mentioned with the time consider the time as 24 hour format.
If diagnosis or history time is not mentioned along with the date, assume default time in strict as 00:00:00 before converting to UTC.

### Timezone Handling:

Try to identify the timezone using:
Explicit timezone mentions like PST, EDT, EST, UTC-5, etc.
Resolve daylight savings time if possible (e.g., EST vs EDT).
If no timezone information is available, default to Eastern Time (ET).

### Output Format:

Convert the resulting datetime to UTC.
Final output should be in format: YYYY-MM-DD HH:mm:ss.
Fill the final output datetime strictly to the corresponding diagnosis or history Date field. If no date provided for the corresponding diagnosis or history, leave the Date field empty.

### Example

Input: “Medication starts on 03/07/2025 in EST.” Processed as: 2025-07-03 00:00:00 EST, Output: 2025-07-03 05:00:00 in UTC

### 3. Missing Information:

If there's no information available for a particular field, leave the Value field empty (e.g., Value: “ ”).

### 4. Formatting the JSON Output:

Your response should only be a JSON that strictly follows the structure of the input JSON template.
JSON formatting should be correct.
Ensure each field in the JSON is filled out correctly based on the information extracted from the text.
If there's no information available for a particular field, leave the Value field empty (e.g., Value: “ ”).
You should not add anything other than the values into the field to the JSON.
Here's an example of your output format:
{
   “Diagnosis”: {
   “description”: “List of the test details related to the current diagnosis (add the detailed information of the test into the Result field)”,
   “DiagnosisList”: [
      {
         “Title”: {
         “Type”: “string”,
         “Value”:
         },
         “Order”: {
         “Type”: “string”,
         “Value”: “Consult eNote”
         },
         “Test”: {
         “Type”: “string”,
         “Value”: “Consult eNote”
         },
         “Date”: {
         “Type”: “date”,
         “Value”: “2024-12-30 21:43:00”
         },
         “Result”: {
         “Type”: “string”,
         “Value”: “Dictated by: Richard Mitchell\nPatient Name: Hirst, Ronald\nSERVICE and LENGTH OF STAY\nService: Orthopedic Surgery\nDate of Admission: 10/21/2024\nHospital Day: #1, Hour #17 (current date and time: 10-22-2024 11:45) . . . ”
         }
         }
      ]
   },
   “Historys”: {
   “description”: “The past medical tests of the patient one by one (add the whole details of the tests done into the Result field for each medical history)”,
   “HistoryList”: [
      {
         “Title”: {
         “Type”: “string”,
         “Value”: “ ”
         },
         “Order”: {
         “Type”: “string”,
         “Value”: “IP-H and P”
         },
         “Test”: {
         “Type”: “string”,
         “Value”: “IP-H and P”
         },
         “Date”: {
         “Type”: “date”,
         “Value”: “2024-12-30 21:43:00”
         },
         “Result”: {
         “Type”: “string”,
         “Value”: “University of Pittsburgh Medical Center\nPatient: SLEDGE, ANTOINETTE\nMRN: 784010833\nFIN: 0001044424284\nAge: 70 years\nSex: Female\nDOB: 5/10/1954\nAssociated Diagnoses: None\nAuthor: EBERSBACHER, ASHTEN MARIE\nBasic Information Visit Information: Patient seen on 10/11/2024.\nHistory of Present Illness: Bob Smith is a 65 yo F w/ hx of obesity, hx craniopharyngioma s/p resection c/b panhypopituitarism, epilepsy, DI on desmopressin, OSA, HFpEF, recurrent DVT/PE, chronic lymphedema who was brought in by EMS due to generally feeling unwell. On my evaluation, she states that she has just felt “unwell” over the past couple of days. Denies any fevers, chills. Says that her right knee is hurting but states that it is because it was because we got her into bed. When asked about pain elsewhere, she states she is having pain all over which brought her in. She denies any dysuria, denies urinary hesitancy, denies abdominal pain. Is not having issues w/ moving her bowels. Denies any new cough or upper respiratory symptoms. Denies shortness of breath. Says she might have a bit more swelling on her legs compared to usual. Denies nausea, vomiting. States that she lives with her son who is her caregiver and she feels safe at home. States that she has been bedridden for the past 1-2 years.”
         }
         }
      ]
   }
}

### Example 5—Medication Prompt

You're an assistant designed to provide information from the content provided by Users.
Users will provide a string of JSON object with “Type”, “value”, you'll respond with the JSON object by following the instructions given below:
provide answer for each question by replacing the “value” field for each object in JSON”,
Each questions answer should be structured according to its type (string, number, date) as specified.
Each questions should be answered according to the details provided in the “description” field.
Fill in the Allergy field with any known allergies related to the medication.
If frequency is listed as clock times, include those details in 24 hour format in the Frequency field.

### Date and Time Handling:

Extract the start date and end date of each medication.

### Date Format:

Return the medication dates in strict YYYY-MM-DD format.

### Time Extraction:

If the time of medication is mentioned along with the date, extract it.
Time can be in 12 hour or 24 hour format. If AM/PM is not mentioned with the time consider that time as 24 hour format.
If medication time is not mentioned along with the date, assume default time in strict as 00:00:00 before converting to UTC.

### Timezone Handling:

Try to identify the timezone using:
Explicit timezone mentions like PST, EDT, EST, UTC-5, etc.
Resolve daylight savings time if possible (e.g., EST vs EDT).
If no timezone information is available, default to Eastern Time (ET).

### Output Format:

Convert the resulting datetime to UTC.
Final output should be in format: YYYY-MM-DD HH:mm:ss.
Fill the final output datetime strictly in the corresponding medication StartDate or EndDate field.
If no date provided for the corresponding medications, leave the StartDate EndDate fields empty.

### Example

Input: “Medication starts on 03/07/2025 in EST.”
Processed as: 2025-07-03 00:00:00 EST, Output: 2025-07-03 05:00:00 in UTC
Extract all the medications given in the context and add it into the JSON.
If not enough information found about the question leave the “Value” field empty.
Here's an example of your output format:
{
   “Medications”: {
      “description”: “List each and every medications and its available details mentioned in the report”,
      “MedicationsList”: [
         {
         “Allergy”: {
         “Type”: “string”,
         “Value”: “No”
         },
         “Dosage”: {
         “Type”: “string”,
         “Value”: “2.5 mg”
         },
         “EndDate”: {
         “Type”: “date”,
         “Value”: “2025-02-28 21:42:00”
         },
         “Frequency”: {
         “Type”: “string”,
         “Value”: “2 times daily 0900,2100”
         },
         “Medication”: {
         “Type”: “string”,
         “Value”: “APIXABAN 2.5 MG TABLET”
         },
         “Note”: {
         “Type”: “string”,
         “Value”:
         },
         “Route”: {
         “Type”: “string”,
         “Value”: “Oral”
         },
         “StartDate”: {
         “Type”: “date”,
         “Value”: “2024-12-30 21:43:00”
         }
         },
         {
         “Allergy”: {
         “Type”: “string”,
         “Value”: “No”
         },
         “Dosage”: {
         “Type”: “string”,
         “Value”: “100 mcg”
         },
         “EndDate”: {
         “Type”: “date”,
         “Value”: “2025-01-01 15:00:00”
         },
         “Frequency”: {
         “Type”: “string”,
         “Value”: “2 times daily 0900,2100”
         },
         “Medication”: {
         “Type”: “string”,
         “Value”: “Asmanex HFA 100 mcg”
         },
         “Note”: {
         “Type”: “string”,
         “Value”:
         },
         “Route”: {
         “Type”: “string”,
         “Value”: “Inhale”
         },
         “StartDate”: {
         “Type”: “date”,
         “Value”: “2024-12-31 15:00:00”
         }
         }
      ]
   }
}

### Example 6: Large File Chunking Customer Defined Prompt

You're an assistant designed to provide information from the content provided by Users.
Users will provide a string of JSON object with “QuestionBankId”, “QuestionType”, “QuestionName”, “Options”, “AiAnswers”, “IsAnswered”, “NestedQuestions” you'll respond following the instructions given below:
Summerise all the important data from the context given by the user,
Copy and paste the part of the context text that might have answers to the Questions in the QuestionName,
Add the page numbers with each summerized line from where the answers where found in the context.

### Example 7: Customer-Defined Prompt

You're an assistant designed to provide information from the content provided by Users. Users provided content will be a collection of summaries made from the chunks of a patients report.
Users will provide a string of JSON object with “QuestionBankId”, “QuestionType”, “QuestionName”, “Options”, “AIAnswers”, “IsAnswered”, “NestedQuestions” you'll respond with the JSON object by following the instructions given below:
provide answer for each question in “QuestionName” by replacing the value in “AIAnswers”, Each question answer should be answered according to its type (1—text, 2—choice, 3—multi select, 4—date) as specified.
For choice, multi select, date leave “AIAnswers” empty if not found enough information found. For choice and multiselect, fill answers only from the given options.
For text QuestionType, answer the questions elaborately as a registered nurse explaining to someone who may not be clinical
Dates should always be in YYYY-MM-DD format.
For text QuestionType, answers should be a single string.
In pageNumber add the corresponding page numbers from which each answers where found.
If NestedQuestions are present in a question, then answer all the nested questions in the NestedAnswers as per the corresponding types provided into the “AiNestedQuestionAnswers”.
If answers are found change the value of “IsAnswered” to true, if not enough information found leave the “AIAnswers” unchanged.
Here's an example of output format:
{
}
   “QuestionBankId”: 102,
   “QuestionType”: 1,
   “QuestionName”: “Referral rationale/diagnosis”,
   “Options”: [ ],
   “AIAnswers”: [“s/p PPM placement d/t symptomatic bradycardia—Z95.0” ],
   “IsAnswered”: true,
   “PageNumber”: [3]
},
{
   “QuestionBankId”: 73,
   “QuestionType”: 2,
   “QuestionName”: “Intellectual disabilities”,
   “Options”: [“Yes”, “No” ],
   “AIAnswers”: [“No” ],
   “IsAnswered”: true,
   “PageNumber”: [5]
},
{
   “QuestionBankId”: 92,
   “QuestionType”: 3,
   “QuestionName”: “Access/Devices”,
   “Options”: [“Peripheral IV”, “PICC”, “PowerPort”, “Port (standard)”, “Pacer” ],
   “AIAnswers”: [“PICC”,“Pacer” ],
   “IsAnswered”: true,
   “PageNumber”: [5,6,7]
},
{
   “QuestionBankId”: 107,
   “QuestionType”: 4,
   “QuestionName”: “Anticipated discharge date”,
   “Options”: [ ],
   “AIAnswers”: [“08/16/24” ],
   “IsAnswered”: true,
   “PageNumber”: [7,19]
},
{
   “QuestionBankId”: 297,
   “QuestionType”: 2,
   “QuestionName”: “Is the patient smoker or vape”,
   “Options”: [“Smoker”,“Vape” ],
   “AIAnswers”: [“Smoker” ],
   “NestedAnswers”: [
      {
      “NestedQuestionId”: 1,
      “NestedQuestionType”: 2,
      “NestedQuestion”: “test question?”,
      “SelectedOption”: [
         “Smoker”
         ],
      “NestedQuestionOptions”: [
         “test option1”,
         “test option2”
         ]
      “AiNestedQuestionAnswers”: [“test option1” ],
      “IsAnswered”: true,
      “PageNumber”: [7,38]
      },
      {
         “NestedQuestionId”: 2,
         “NestedQuestionType”: 2,
         “NestedQuestion”: “Test question?”,
         “SelectedOption”:
         “Vape”
         ],
         “NestedQuestionOptions”: [
         “test option1”,
         “test option2”
         ]
         “AiNestedQuestionAnswers”: [“test option2” ],
         “IsAnswered”: true,
         “PageNumber”: [5,8]
         }
      ]
   ]
}
]
Note that if not enough information from the users provided context is found to answer a question accurately, leave the “AI Answers” Empty and mark “Is Answered” to false. For choice and multiselect, fill answers only from the given options.

### EXEMPLARY SOLUTIONS

Some embodiments may implement one or more of the following exemplary solutions, listed in clause-format. The following clauses are supported and further described in the embodiments above and throughout this document.

Solution 1. A system, comprising: a network interface configured to receive input data streams from multiple source nodes; at least one processor coupled to the network interface; memory coupled to the at least one processor, wherein the memory stores instructions that, when executed by the processor, cause the system to perform operations including: receiving, via the network interface, a data package from a source node; generating, using a machine learning component, an analysis record based on the data package, wherein the analysis record comprises compatibility indicators; identifying, from a plurality of service providing nodes, a candidate node by comparing the compatibility indicators with node capability specifications; and generating a recommendation including the candidate node for the source node.

Solution 2. The system of any one or more of Solution 1 or any other solutions disclosed herein, wherein: the operations further comprise extracting text from the data package; and generating the analysis record comprises processing the extracted text using the machine learning component.

Solution 3. The system of any one or more of Solution 2 or any other solutions disclosed herein, wherein extracting text from the data package comprises performing optical character recognition (OCR) on the data package.

Solution 4. The system of any one or more of Solution 2 or any other solutions disclosed herein, wherein the machine learning component comprises a natural language processing (NLP) engine.

Solution 5. The system of any one or more of Solution 4 or any other solutions disclosed herein, wherein the NLP engine is configured to use a large language model (LLM) to analyze the extracted text.

Solution 6. A computer-implemented method, comprising: receiving, via a network interface, a data package from a source node; generating, using a machine learning component, an analysis record based on the data package, wherein the analysis record comprises compatibility indicators; identifying, from a plurality of service providing nodes, a candidate node by comparing the compatibility indicators with node capability specifications, and generating a recommendation including the candidate node for the source node.

Solution 7. The method of any one or more of Solution 6 or any other solutions disclosed herein, comprising: extracting text from the data package, wherein generating the analysis record comprises processing the extracted text using the machine learning component.

Solution 8. The method of any one or more of Solution 7 or any other solutions disclosed herein, wherein extracting text from the data package comprises performing optical character recognition (OCR) on the data package.

Solution 9. The method of any one or more of Solution 7 or any other solutions disclosed herein, wherein the machine learning component comprises a natural language processing (NLP) engine.

Solution 10. The method of any one or more of Solution 9 or any other solutions disclosed herein, wherein the NLP engine is configured to use a large language model (LLM) to analyze the extracted text.

Solution 11. The method of any one or more of Solution 7 or any other solutions disclosed herein, comprising: identifying keywords or section of interest by analyzing the extracted text.

Solution 12. The method of any one or more of Solution 11 or any other solutions disclosed herein, wherein identifying keywords or section of interest comprises analyzing the extracted text by the machine learning component.

Solution 13. The method of any one or more of Solution 11 or any other solutions disclosed herein, wherein processing the data package further comprises: applying visual indicators to the identified keywords or sections of interest for presentation in a display of the extracted text or the data package.

Solution 14. The method of any one or more of Solution 13 or any other solutions disclosed herein, wherein the visual indicators comprise at least one of: color coding, font modifications, or background highlighting.

Solution 15. The method of any one or more of Solution 13 or any other solutions disclosed herein, wherein applying the visual indicators to the identified keywords or sections of interest comprises: categorizing the keywords or sections of interest into multiple categories; assigning different visual indicators to different categories; and labelling the keywords or sections of interest with their corresponding visual indicators.

Solution 16. The method of any one or more of Solution 15 or any other solutions disclosed herein, wherein: the multiple categories comprise clinical categories; and the visual indicators are configured to integrate with a clinical review interface.

Solution 17. The method of any one or more of Solution 7 or any other solutions disclosed herein, wherein generating the analysis record comprises: generating, using the machine learning component, proposed responses to predefined queries based on the extracted text.

Solution 18. The method of any one or more of Solution 17 or any other solutions disclosed herein, wherein generating the analysis record further comprises at least one of: providing source attribution linking the proposed responses to corresponding portions of the data package; or providing status indicators indicating whether each response was generated by the machine learning component.

Solution 19. The method of any one or more of Solution 18 or any other solutions disclosed herein, wherein: the predefined queries comprise clinical review questions; the proposed responses comprise automatically generated clinical assessments; and the source attribution links to specific portions of medical documentation supporting each assessment.

Solution 20. The method of any one or more of Solution 17 or any other solutions disclosed herein, comprising: transmitting the data package or the proposed responses to predefined queries to a user for review; receiving, from the user, user input comprising a modification to the proposed responses; and generating the analysis record based on the modification.

Solution 21. The method of any one or more of Solution 6 or any other solutions disclosed herein, further comprising: comparing the compatibility indicators with node capability specifications of a first node; determining that the first node has incompatible capability specifications based on the comparison; and designating the first node as not the candidate node.

Solution 22. The method of any one or more of Solution 21 or any other solutions disclosed herein, comprising: identifying multiple candidate nodes.

Solution 23. The method of any one or more of Solution 22 or any other solutions disclosed herein, comprising: ranking the multiple candidate nodes based on at least one factor including distance between the source node and each of the multiple candidate nodes, current processing load of each of the multiple candidate nodes, or an urgency indicator.

Solution 24. The method of any one or more of Solution 22 or any other solutions disclosed herein, further comprising: transmitting, via the network interface, an invitation relating to the data package to each of the multiple candidate nodes.

Solution 25. The method of any one or more of Solution 22 or any other solutions disclosed herein, wherein the multiple candidate nodes collectively satisfy the compatibility indicators.

Solution 26. The method of any one or more of Solution 25 or any other solutions disclosed herein, wherein the multiple candidate nodes are located within a predefined geographical range.

Solution 27. The method of any one or more of Solution 6 or any other solutions disclosed herein, comprising: receiving multiple data packages from one or more source nodes; determining a priority score for each of the multiple data packages; and ordering processing of the multiple data packages based on the priority scores.

Solution 28. The method of any one or more of Solution 27 or any other solutions disclosed herein, wherein the priority score for one of the multiple data packages is determined based on at least one of a medical condition urgency indicator, service providing node proximity, care facility occupancy, care facility specialization alignment, or scarcity of supply for a medical care.

Solution 29. The method of any one or more of Solution 27 or any other solutions disclosed herein, wherein determining a priority score for each of the multiple packages further comprises: adjusting the priority score of a data package based on elapsed time since receiving the data package.

Solution 30. The method of any one or more of Solution 6 or any other solutions disclosed herein, comprising: generating a node-tailored data package for the candidate node.

Solution 31. The method of any one or more of Solution 6 or any other solutions disclosed herein, wherein: the source node comprises a referral source; the data package comprises a referral package relating to a service recipient; and the candidate node comprises a service providing community.

Solution 32. The method of any one or more of Solution 31 or any other solutions disclosed herein, wherein: the analysis record comprises patient care needs; and the compatibility indicators comprise medical care capacities corresponding to the patient care needs.

Solution 33. The method of any one or more of Solution 6 or any other solutions disclosed herein, comprising: retrieve, via an application programming interface (API), information relating to the data package; and generating a notification based on the retrieved information.

Solution 34. The method of any one or more of Solution 33 or any other solutions disclosed herein, wherein the information comprises at least one of: insurance verification data or background check data.

Solution 35. The method of any one or more of Solution 6 or any other solutions disclosed herein, comprising: maintaining a database of historical processing outcomes; receiving an indication of a processing issue for the data package; storing details of the processing issue in the database; and generating an alert for a subsequent data package that matches characteristics of the stored processing issue.

Solution 36. The method of any one or more of Solution 35 or any other solutions disclosed herein, wherein: the historical processing outcomes comprise patient admission records; the processing issue comprises a documented admission concern; and the alert is for a clinical reviewer of previous admission issues.

Solution 37. The method of any one or more of Solution 35 or any other solutions disclosed herein, wherein the database comprises an organization-wide high-risk patient list.

Solution 38. The method of any one or more of Solution 6 or any other solutions disclosed herein, wherein comparing the compatibility indicators comprises: assigning a first indicator level indicating full compatibility; assigning a second indicator level indicating potential compatibility requiring additional verification; and assigning a third indicator level indicating incompatibility.

Solution 39. The method of any one or more of Solution 38 or any other solutions disclosed herein, wherein: the first indicator level corresponds to full alignment with care facility capabilities; the second indicator level indicates need for additional clinical review; and the third indicator level indicates clinical capability mismatch.

Solution 40. The method of any one or more of Solution 6 or any other solutions disclosed herein, comprising: receiving, from the source node or a target node, a notification comprising acceptance or denial of an invitation to the target node in relation to the recommendation, wherein the recommendation comprises one or more candidate nodes including the target node.

Solution 41. The method of any one or more of Solution 40 or any other solutions disclosed herein, further comprising: updating, in real-time, a centralized status database regarding a status of the target node based on the notification.

Solution 42. A system, comprising: a network interface configured to receive an electronic data package from a source node; at least one storage device storing computer-executable instructions; and at least one processor coupled to the at least one storage device and configured to execute the computer-executable instructions to perform operations comprising: obtaining a document image derived from the electronic data package; generating extracted text and location data from the document image, the location data defining mapped locations of the extracted text within the document image; generating, using a machine learning (ML) component, a structured response by: generating a prompt payload comprising at least a portion of the extracted text and a node capability specification of a service providing node; providing the prompt payload to a generative AI model of the ML component; and receiving the structured response from the generative AI model, wherein the structured response comprises: (i) at least one compatibility indicator determined based on the capability specification of the service providing node, and (ii) a source citation identifying at least one relevant text chunk from the extracted text that supports the at least one compatibility indicator; determining whether the at least one compatibility indicator satisfies the node capability specification of the service providing node; and generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

Solution 43. The system of any one or more of Solution 42 or any other solutions disclosed herein, wherein: the node capability specification defines a clinical question and a set of valid answer options for the clinical question, and the generating the prompt payload comprises incorporating the set of valid answer options in the prompt payload to constrain the structured response of the generative AI model to a selected option from the set of valid answer options.

Solution 44. The system of any one or more of Solution 42 or any other solutions disclosed herein, wherein the at least one processor is configured to perform the generating the verification interface using a serverless function trigger activated based on receipt of the electronic data package via the network interface.

Solution 45. The system of any one or more of Solution 42 or any other solutions disclosed herein, wherein: the location data comprises location tags assigned to discrete chunks of the extracted text; the generating the prompt payload comprises embedding the location tags within the extracted text; the source citation received from the generative AI model references a specific location tag; and the generating the verification interface comprises mapping the specific location tag to geometric coordinates for the visual overlay.

Solution 46. The system of any one or more of Solution 42 or any other solutions disclosed herein, wherein: the determining whether the at least one compatibility indicator satisfies the node capability specification comprises determining a recommendation regarding the service providing node; and the verification interface is configured to display the recommendation to a reviewer and receive an input in response to the recommendation.

Solution 47. A method, comprising: receiving, by at least one processor via a network interface, an electronic data package from a source node; obtaining a document image derived from the electronic data package; generating extracted text and location data from the document image, the location data defining mapped locations of the extracted text within the document image; generating, using a machine learning (ML) component, a structured response by: generating a prompt payload comprising at least a portion of the extracted text and a node capability specification of a service providing node; providing the prompt payload to a generative AI model of the ML component; and receiving the structured response from the generative AI model, wherein the structured response comprises: (i) at least one compatibility indicator determined based on the node capability specification of the service providing node, and (ii) a source citation identifying at least one relevant text chunk from the extracted text that supports the at least one compatibility indicator; determining whether the at least one compatibility indicator satisfies the node capability specification of the service providing node; and generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

Solution 48. The method of any one or more of Solution 47 or any other solutions disclosed herein, wherein: the node capability specification defines a clinical question and a set of valid answer options for the clinical question, and the generating the prompt payload comprises incorporating the set of valid answer options in the prompt payload to constrain the structured response of the generative AI model to a selected option from the set of valid answer options.

Solution 49. The method of any one or more of Solution 47 or any other solutions disclosed herein, wherein the visual overlay comprises a bounding box or a color highlight.

Solution 50. The method of any one or more of Solution 47 or any other solutions disclosed herein, wherein: the determining whether the at least one compatibility indicator satisfies the node capability specification comprises determining a recommendation regarding the service providing node; and the generating the verification interface comprises configuring the verification interface to display the recommendation to a reviewer and receive an input in response to the recommendation.

Solution 51. The method of any one or more of Solution 50 or any other solutions disclosed herein, wherein: the recommendation comprises a rejection of the service providing node; and the method comprises causing, in the verification interface, the rejection to be displayed in association with a specific text chunk of the at least one text chunk as evidence for the rejection.

Solution 52. The method of any one or more of Solution 51 or any other solutions disclosed herein, wherein: the causing the rejection to be displayed in association with the specific text chunk comprises causing at least one of the specific text chunk or a link to the specific text chunk to be displayed.

Solution 53. The method of any one or more of Solution 47 or any other solutions disclosed herein, comprising: determining that the at least one compatibility indicator indicates the service providing node is incompatible with the electronic data package; identifying a second service providing node having a node capability specification that is compatible with the at least one compatibility indicator; and generating a second recommendation to route the electronic data package to the second service providing node.

Solution 54. The method of any one or more of Solution 47 or any other solutions disclosed herein, wherein: the location data comprises location tags assigned to discrete text chunks of the extracted text; the generating the prompt payload comprises embedding the location tags within the extracted text; the source citation received from the generative AI model references a specific location tag assigned to the at least one relevant text chunk; and the generating the verification interface comprises mapping the specific location tag to geometric coordinates for the visual overlay.

Solution 55. The method of any one or more of Solution 47 or any other solutions disclosed herein, wherein: the structured response comprises content directly extracted from the electronic data package and AI-generated content; and the verification interface comprises a visual indication distinguishing the AI-generated content from the content directly extracted from the electronic data package.

Solution 56. The method of any one or more of Solution 47 or any other solutions disclosed herein, comprising: determining, based on the structured response, that the electronic data package is incomplete regarding a mandatory data element defined in the node capability specification; and automatically triggering an electronic task to request additional information from the source node regarding the mandatory data element prior to determining whether the at least one compatibility indicator satisfies the node capability specification.

Solution 57. The method of any one or more of Solution 47 or any other solutions disclosed herein, comprising: executing a data sanitization process on the extracted text by masking a predefined type of personal information in the electronic data package.

Solution 58. The method of any one or more of Solution 47 or any other solutions disclosed herein, wherein: the generating the prompt payload comprises including a clinical authorization instruction in the prompt payload configured to override a default content moderation filter of the generative AI model, thereby enabling the generative AI model to process valid clinical descriptions of sensitive medical conditions without censorship.

Solution 59. The method of any one or more of Solution 47 or any other solutions disclosed herein, comprising storing the prompt payload, the structured response, and the source citation in a log to generate an audit trail.

Solution 60. The method of any one or more of Solution 47 or any other solutions disclosed herein, wherein the electronic data package comprises one or more files or data streams selected from the group consisting of: a Portable Document Format (PDF) file, a Facsimile (Fax) image, a rasterized image file, an Extensible Markup Language (XML) file, a Health Level Seven (HL7) message, and a JavaScript Object Notation (JSON) packet.

Solution 61. The method of any one or more of Solution 47 or any other solutions disclosed herein, wherein: the node capability specification defines a plurality of clinical questions; the generating the prompt payload comprises aggregating the plurality of clinical questions and the extracted text into a single prompt payload to execute a single inference pass by the generative AI model; and receiving the structured response comprises receiving a single JavaScript Object Notation (JSON) packet containing, for each of the plurality of clinical questions: (i) a specific compatibility indicator of the at least one compatibility indicator, (ii) an answer determined based on a portion of the at least one relevant text chunk, and (iii) a portion of the source citation corresponding to the portion of the at least one relevant text chunk in support of the compatibility indicator.

Solution 62. The method of any one or more of Solution 47 or any other solutions disclosed herein, wherein: the location data comprises structural metadata tags associated with geometric coordinates for discrete elements of the extracted text; the source citation received from the generative AI model identifies the at least one relevant text chunk using specific structural metadata tags; and the generating the verification interface comprises using the location data to map the specific structural metadata tags to the geometric coordinates to render the visual overlay.

Solution 63. The method of any one or more of Solution 47 or any other solutions disclosed herein, wherein: the visual overlay is dynamically rendered upon user interaction with the at least one compatibility indicator.

Solution 64. The method of any one or more of Solution 47 or any other solutions disclosed herein, wherein: the deriving the at least one compatibility indicator comprises assigning a confidence score to the at least one compatibility indicator, and the verification interface visually distinguishes one of the at least one compatibility indicator that has a confidence score below a threshold.

Solution 65. The method of any one or more of Solution 47 or any other solutions disclosed herein, comprising: transmitting the structured response to an external electronic health record (EHR) system.

Solution 66. A system, comprising: memory storing computer-readable instructions; one or more processors that when executing the computer-readable instructions, are configured to perform the method of any one or more examples disclosed herein.

Solution 67. One or more non-transitory computer-readable media storing computer-executable instructions that, when executed by one or more processors of a wireless communication device, cause the one or more processors to perform the method of any one or more examples disclosed herein.

The above description and drawings are illustrative and are not to be construed as limiting. Numerous specific details are described to provide a thorough understanding of the disclosure. However, in some instances, well-known details are not described in order to avoid obscuring the description. Further, various modifications may be made without deviating from the scope of the embodiments.

Reference in this specification to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

The terms used in this specification generally have their ordinary meanings in the art, within the context of the disclosure, and in the specific context where each term is used. It will be appreciated that the same thing can be said in more than one way. Consequently, alternative language and synonyms may be used for any one or more of the terms discussed herein, and any special significance is not to be placed upon whether or not a term is elaborated or discussed herein. Synonyms for some terms are provided. A recital of one or more synonyms does not exclude the use of other synonyms. The use of examples anywhere in this specification, including examples of any term discussed herein, is illustrative only and is not intended to further limit the scope and meaning of the disclosure or of any exemplified term. Likewise, the disclosure is not limited to various embodiments given in this specification. Unless otherwise defined, all technical and scientific terms used herein have the same meaning as commonly understood by one of ordinary skill in the art to which this disclosure pertains. In the case of conflict, the present document, including definitions, will control.

What is claimed is:

1. A system, comprising:
a network interface configured to receive an electronic data package from a source node;
at least one storage device storing computer-executable instructions; and
at least one processor coupled to the at least one storage device and configured to execute the computer-executable instructions to perform operations comprising:
obtaining a document image derived from the electronic data package;
generating extracted text and location data from the document image, the location data defining mapped locations of the extracted text within the document image;
generating, using a machine learning (ML) component, a structured response by:
generating a prompt payload comprising at least a portion of the extracted text and a node capability specification of a service providing node;
providing the prompt payload to a generative AI model of the ML component; and
receiving the structured response from the generative AI model, wherein the structured response comprises:
(i) at least one compatibility indicator determined based on the capability specification of the service providing node, and
(ii) a source citation identifying at least one relevant text chunk from the extracted text that supports the at least one compatibility indicator;
determining whether the at least one compatibility indicator satisfies the node capability specification of the service providing node; and
generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

2. The system of claim 1, wherein:
the node capability specification defines a clinical question and a set of valid answer options for the clinical question, and
the generating the prompt payload comprises incorporating the set of valid answer options in the prompt payload to constrain the structured response of the generative AI model to a selected option from the set of valid answer options.

3. The system of claim 1, wherein the at least one processor is configured to perform the generating the verification interface using a serverless function trigger activated based on receipt of the electronic data package via the network interface.

4. The system of claim 1, wherein:
the location data comprises location tags assigned to discrete chunks of the extracted text;
the generating the prompt payload comprises embedding the location tags within the extracted text;
the source citation received from the generative AI model references a specific location tag; and
the generating the verification interface comprises mapping the specific location tag to geometric coordinates for the visual overlay.

5. The system of claim 1, wherein:
the determining whether the at least one compatibility indicator satisfies the node capability specification comprises determining a recommendation regarding the service providing node; and
the verification interface is configured to display the recommendation to a reviewer and receive an input in response to the recommendation.

6. A method, comprising:
receiving, by at least one processor via a network interface, an electronic data package from a source node;
obtaining a document image derived from the electronic data package;
generating extracted text and location data from the document image, the location data defining mapped locations of the extracted text within the document image;
generating, using a machine learning (ML) component, a structured response by:
generating a prompt payload comprising at least a portion of the extracted text and a node capability specification of a service providing node;
providing the prompt payload to a generative AI model of the ML component; and
receiving the structured response from the generative AI model, wherein the structured response comprises:
(i) at least one compatibility indicator determined based on the node capability specification of the service providing node, and
(ii) a source citation identifying at least one relevant text chunk from the extracted text that supports the at least one compatibility indicator;
determining whether the at least one compatibility indicator satisfies the node capability specification of the service providing node; and
generating a verification interface for presentation, wherein the verification interface comprises a visual overlay that visually associates, based on at least one of the source citation or the mapped location of the at least one relevant text chunk, the at least one compatibility indicator with the at least one relevant text chunk on the document image.

7. The method of claim 6, wherein:
the node capability specification defines a clinical question and a set of valid answer options for the clinical question, and
the generating the prompt payload comprises incorporating the set of valid answer options in the prompt payload to constrain the structured response of the generative AI model to a selected option from the set of valid answer options.

8. The method of claim 6, wherein the visual overlay comprises a bounding box or a color highlight.

9. The method of claim 6, wherein:
the determining whether the at least one compatibility indicator satisfies the node capability specification comprises determining a recommendation regarding the service providing node; and
the generating the verification interface comprises configuring the verification interface to display the recommendation to a reviewer and receive an input in response to the recommendation.

10. The method of claim 9, wherein:
the recommendation comprises a rejection of the service providing node; and
the method comprises causing, in the verification interface, the rejection to be displayed in association with a specific text chunk of the at least one text chunk as evidence for the rejection.

11. The method of claim 10, wherein:
the causing the rejection to be displayed in association with the specific text chunk comprises causing at least one of the specific text chunk or a link to the specific text chunk to be displayed.

12. The method of claim 6, comprising:
determining that the at least one compatibility indicator indicates the service providing node is incompatible with the electronic data package;
identifying a second service providing node having a node capability specification that is compatible with the at least one compatibility indicator; and
generating a second recommendation to route the electronic data package to the second service providing node.

13. The method of claim 6, wherein:
the location data comprises location tags assigned to discrete text chunks of the extracted text;
the generating the prompt payload comprises embedding the location tags within the extracted text;
the source citation received from the generative AI model references a specific location tag assigned to the at least one relevant text chunk; and
the generating the verification interface comprises mapping the specific location tag to geometric coordinates for the visual overlay.

14. The method of claim 6, wherein:
the structured response comprises content directly extracted from the electronic data package and AI-generated content; and
the verification interface comprises a visual indication distinguishing the AI-generated content from the content directly extracted from the electronic data package.

15. The method of claim 6, comprising:
determining, based on the structured response, that the electronic data package is incomplete regarding a mandatory data element defined in the node capability specification; and
automatically triggering an electronic task to request additional information from the source node regarding the mandatory data element prior to determining whether the at least one compatibility indicator satisfies the node capability specification.

16. The method of claim 6, comprising:
executing a data sanitization process on the extracted text by masking a predefined type of personal information in the electronic data package.

17. The method of claim 6, wherein:
the generating the prompt payload comprises including a clinical authorization instruction in the prompt payload configured to override a default content moderation filter of the generative AI model, thereby enabling the generative AI model to process valid clinical descriptions of sensitive medical conditions without censorship.

18. The method of claim 6, comprising storing the prompt payload, the structured response, and the source citation in a log to generate an audit trail.

19. The method of claim 6, wherein the electronic data package comprises one or more files or data streams selected from the group consisting of: a Portable Document Format (PDF) file, a Facsimile (Fax) image, a rasterized image file, an Extensible Markup Language (XML) file, a Health Level Seven (HL7) message, and a JavaScript Object Notation (JSON) packet.

20. The method of claim 6, wherein:
the node capability specification defines a plurality of clinical questions;
the generating the prompt payload comprises aggregating the plurality of clinical questions and the extracted text into a single prompt payload to execute a single inference pass by the generative AI model; and
receiving the structured response comprises receiving a single JavaScript Object Notation (JSON) packet containing, for each of the plurality of clinical questions: (i) a specific compatibility indicator of the at least one compatibility indicator, (ii) an answer determined based on a portion of the at least one relevant text chunk, and (iii) a portion of the source citation corresponding to the portion of the at least one relevant text chunk in support of the compatibility indicator.

21. The method of claim 6, wherein:
the location data comprises structural metadata tags associated with geometric coordinates for discrete elements of the extracted text;
the source citation received from the generative AI model identifies the at least one relevant text chunk using specific structural metadata tags; and
the generating the verification interface comprises using the location data to map the specific structural metadata tags to the geometric coordinates to render the visual overlay.

22. The method of claim 6, wherein:
the visual overlay is dynamically rendered upon user interaction with the at least one compatibility indicator.

23. The method of claim 6, wherein:
the deriving the at least one compatibility indicator comprises assigning a confidence score to the at least one compatibility indicator, and
the verification interface visually distinguishes one of the at least one compatibility indicator that has a confidence score below a threshold.

24. The method of claim 6, comprising:
transmitting the structured response to an external electronic health record (EHR) system.

25. At least one non-transitory computer-readable storage medium storing instructions that, when executed by at least one processor, cause the at least one processor to perform operations comprising:
receiving, by the at least one processor via a network interface, an electronic data package from a source node;
obtaining a document image derived from the electronic data package;
generating extracted text and location data of the document image, the location data defining mapped locations of the extracted text within the document image;
generating, using a machine learning (ML) component, a structured response by:
generating a prompt payload comprising at least a portion of the extracted text and a node capability specification of a service providing node;
providing the prompt payload to a generative AI model of the ML component; and
receiving the structured response from the generative AI model, wherein the structured response comprises:
(i) at least one compatibility indicator determined based on the capability specification of the service providing node, and
(ii) a source citation identifying at least one relevant text chunk from the extracted text that supports the at least one compatibility indicator;
determining whether the at least one compatibility indicator satisfies the node capability specification of the service providing node; and
generating a verification interface for the source node, wherein the verification interface comprises a visual overlay that visually associates, based on at least one of the source citation or the mapped location of the at least one relevant text chunk, the at least one compatibility indicator with the at least one relevant text chunk on the document image.
